(19) United States
(12) Patent Application Publication
(10) Pub. No.: US 2023/0406861 A1
(43) Pub. Date: Dec. 21, 2023

(54) ORGANIC MOLECULES FOR OPTOELECTRONIC DEVICES

(71) Applicant: CYNORA GMBH, Bruchsal (DE)

(72) Inventor: Ramin PASHAZADEH, Bruchsal (DE)

(21) Appl. No.: 18/029,625

(22) PCT Filed: Sep. 28, 2021
(86) PCT No.: PCT/EP2021/076611

§ 371 Date: Mar. 30, 2023

(30) Foreign Application Priority Data
Oct. 2, 2020 (EP) 20199796.2

(51) Int. Cl. C07D 487/22; H10K 50/11; C09K 11/06; H10K 85/60; C07D 471/22; C07D 498/22; C07D 513/22

(52) U.S. Cl. CPC C07D 487/22; H10K 50/11; C09K 11/06; H10K 85/6572; C07D 471/22; C07D 498/22; C07D 513/22; H10K 85/654; C09K 2211/1081; C09K 2211/1077; H10K 2101/20

(57) ABSTRACT

The invention relates to an organic molecule for optoelectronic devices. According to the invention, the organic molecule has a structure of Formula I:

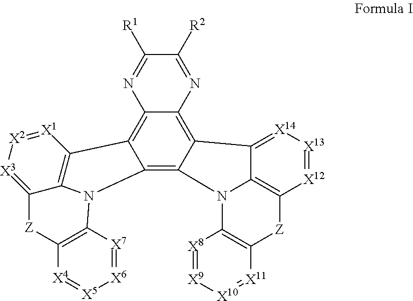

wherein
X¹, X², X³, X⁴, X⁵, X⁶, X⁷, X⁸, X⁹, X¹⁰, X¹¹, X¹², X¹³, and X¹⁴ is independently selected from the group consisting of N and CR^{a}; and
Z is at each occurrence independently selected from the group consisting of a direct bond, CR³R⁴, C═CR³R⁴, C═O, C═NR³, NR³, O, SiR³R⁴, S, S(O), and S(O)₂.

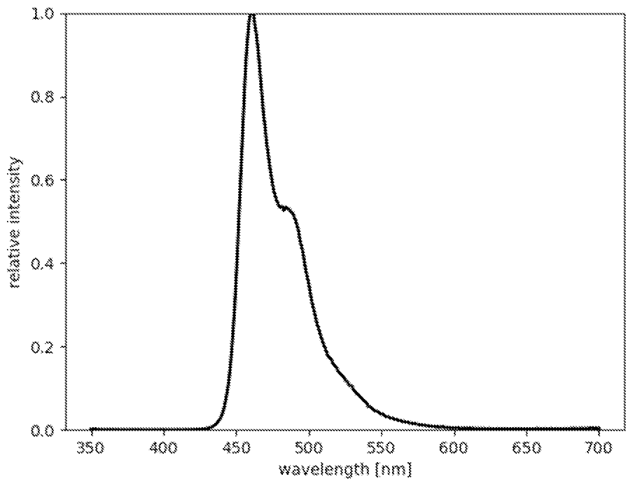

### CROSS-REFERENCE TO RELATED APPLICATIONS

This application is a U.S. National Phase patent application of International Patent Application Number PCT/EP2021/076611, filed on Sep. 28, 2021, which claims priority to European Patent Application Number 20199796.2, filed on Oct. 2, 2020, the entire content of each of which is incorporated herein by reference.

### BACKGROUND

The invention relates to organic light-emitting molecules and their use in organic light-emitting diodes (OLEDs) and in other optoelectronic devices.

### DESCRIPTION

The object of the present invention is to provide molecules which are suitable for use in optoelectronic devices.

This object is achieved by the invention which provides a new class of organic molecules.

The organic molecules of the invention are purely organic molecules, i.e., they do not contain any metal ions in contrast to metal complexes known for the use in optoelectronic devices. The organic molecules of the invention, however, may include metalloids, in particular B, Si, Sn, Se, and/or Ge.

The organic molecules of the invention exhibit emission maxima in the blue, sky-blue, green, or yellow spectral range. The organic molecules exhibit in particular emission maxima between 420 nm and 520 nm, preferably between 440 nm and 495 nm, more preferably between 450 nm and 470 nm or the organic molecules exhibit in particular emission maxima between 490 and 600 nm, more preferably between 510 and 560 nm, and even more preferably between 520 and 540 nm. The photoluminescence quantum yields of the organic molecules according to the invention are, in particular, 50% or more. The use of the molecules according to the invention in an optoelectronic device, for example an organic light-emitting diode (OLED), leads to higher efficiencies or higher color purity, expressed by the full width at half maximum (FWHM) of an emission spectrum, of the device. Corresponding OLEDs have a higher stability than OLEDs with known emitter materials and comparable color.

The organic light-emitting molecules according to the invention include or consist of a structure of Formula I:

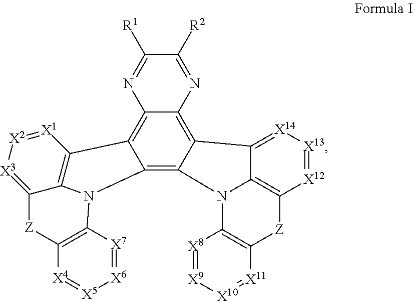

wherein
X¹, X², X³, X⁴, X⁵, X⁶, X⁷, X⁸, X⁹, X¹⁰, X¹¹, X¹², X¹³, and X¹⁴ is independently from each other selected from the group consisting of N and CR^{a};
Z is at each occurrence independently from one another selected from the group consisting of a direct bond, CR³R⁴, C═CR³R⁴, C═O, C═NR³, NR³, O, SiR³R⁴, S, S(O) and S(O)₂;
R¹ and R² is at each occurrence independently from one another selected from the group consisting of hydrogen; deuterium; N(R⁵)₂; OR⁵; Si(R⁵)₃; B(OR⁵)₂; OSO₂R⁵; CF₃; CN; F; Br; I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR^{S5};
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵; and
a mono- or polycyclic, aliphatic, aromatic and/or benzo-fused ring system formed by ring-closure with one or more of the other substituents selected from the group consisting of R¹, R² and R⁵;
R^{a}, R³ and R⁴ is at each occurrence independently from one another selected from the group consisting of hydrogen;
deuterium;
N(R⁵)₂;
OR⁵;
Si(R⁵)₃;
B(OR⁵)₂;
OSO₂R⁵;
CF₃;
CN;
F;
Br;
I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵;
R⁵ is at each occurrence independently from one another selected from the group consisting of hydrogen; deuterium; N(R⁶)₂; OR; Si(R⁶)₃; B(OR⁶)₂; OSO₂R⁶; CF₃; CN; F; Br; I;
C₁-C₄₀-alkyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-alkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-thioalkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkenyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkynyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₆-C₆₀-aryl,
which is optionally substituted with one or more substituents R⁶; and
C₃-C₅₇-heteroaryl,
which is optionally substituted with one or more substituents R⁶;
R⁶ is at each occurrence independently from one another selected from the group consisting of hydrogen; deuterium; OPh (Ph=phenyl); CF₃; CN; F;
C₁-C₅-alkyl,
wherein one or more hydrogen atoms are optionally, independently from each other substituted by deuterium, CN, CF₃, or F;
C₁-C₅-alkoxy,
wherein one or more hydrogen atoms are optionally, independently from each other substituted by deuterium, CN, CF₃, or F;
C₁-C₅-thioalkoxy,
wherein one or more hydrogen atoms are optionally, independently from each other substituted by deuterium, CN, CF₃, or F;
C₂-C₅-alkenyl,
wherein one or more hydrogen atoms are optionally, independently from each other substituted by deuterium, CN, CF₃, or F;
C₂-C₅-alkynyl,
wherein one or more hydrogen atoms are optionally, independently from each other substituted by deuterium, CN, CF₃, or F;
C₆-C₁₈-aryl,
which is optionally substituted with one or more C₁-C₅-alkyl substituents;
C₃-C₁₇-heteroaryl,
which is optionally substituted with one or more C₁-C₅-alkyl substituents;
N(C₆-C₁₈-aryl)₂;
N(C₃-C₁₇-heteroaryl)₂; and
N(C₃-C₁₇-heteroaryl)(C₆-C₁₈-aryl);
wherein the substituents R¹, R², R^{a}, R³, R⁴ or R⁵ independently from each other optionally form a mono- or polycyclic, aliphatic, aromatic and/or benzo-fused ring system with one or more substituents R¹, R², R^{a}, R³, R⁴ or R⁵.

Selected examples of organic molecules according to the invention are shown in the following:

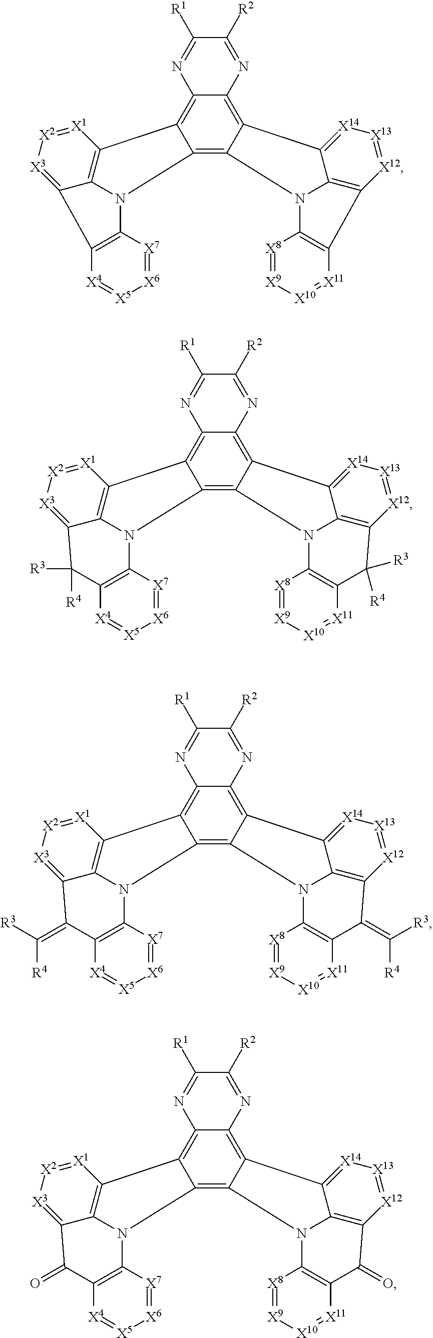

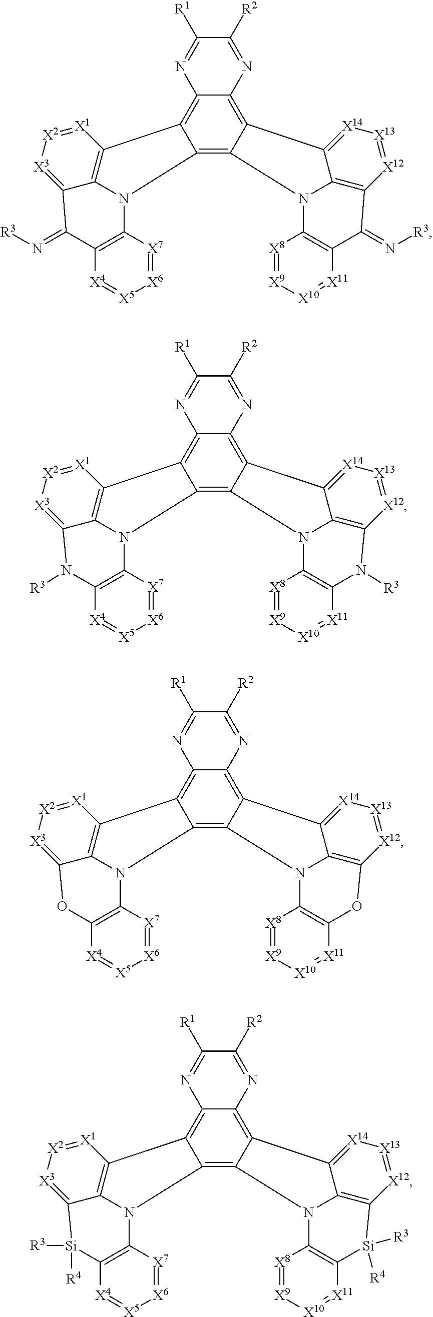

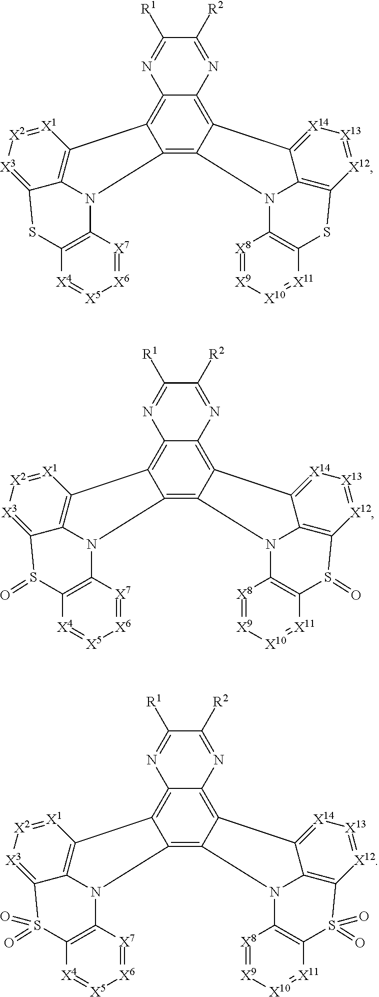

In one embodiment, the organic molecules according to the invention include or consist of a structure of Formula Ia:

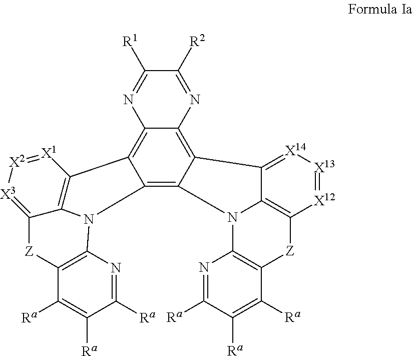

wherein
X¹, X², X³, X¹², X¹³, and X¹⁴ is independently from each other selected from the group consisting of N and CR^{a}.

In one embodiment of the invention, the organic molecules according to the invention include or consist of a structure of Formula Ib:

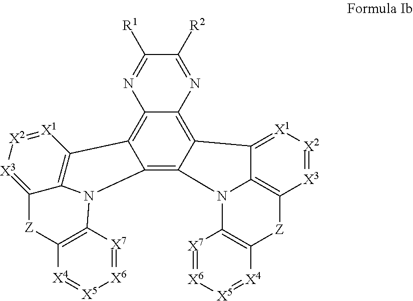

wherein X¹, X², X³, X⁴, X⁵, X⁶ and X⁷ is independently from each other selected from the group consisting of N and CR^{a}.

In one embodiment of the invention, the organic molecules according to the invention include or consist of a structure selected from the group consisting of Formula IIa, Formula IIb, Formula IIc, Formula IId, and Formula IIe:

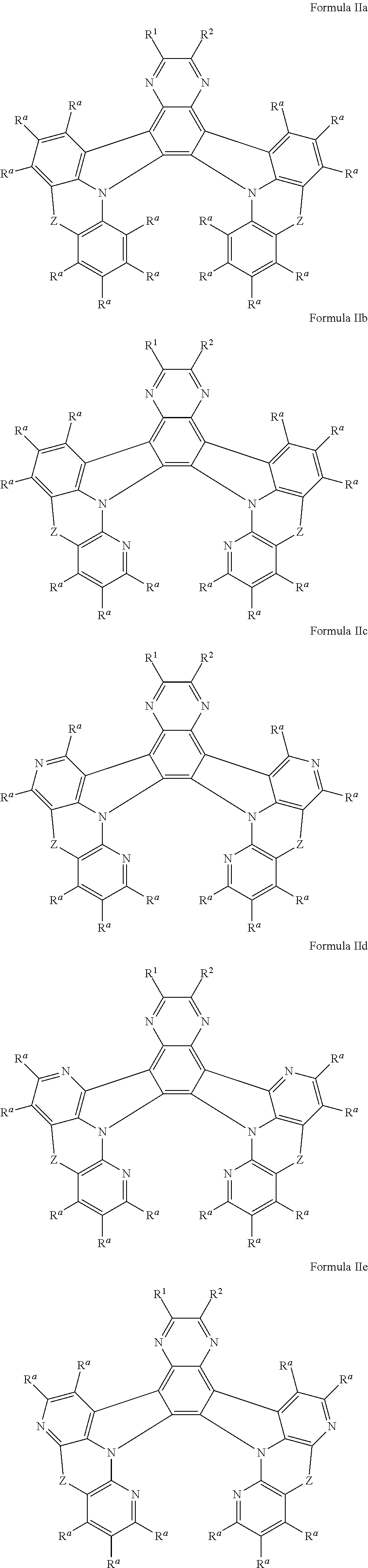

In a preferred embodiment, the organic molecules according to the invention include or consist of a structure according to Formula IIa.

In a preferred embodiment of the invention, the organic molecules according to the invention include or consist of a structure selected from the group consisting of Formula IIa-1, Formula IIb-1, Formula IIc-1, Formula IId-1, and Formula IIe-1:

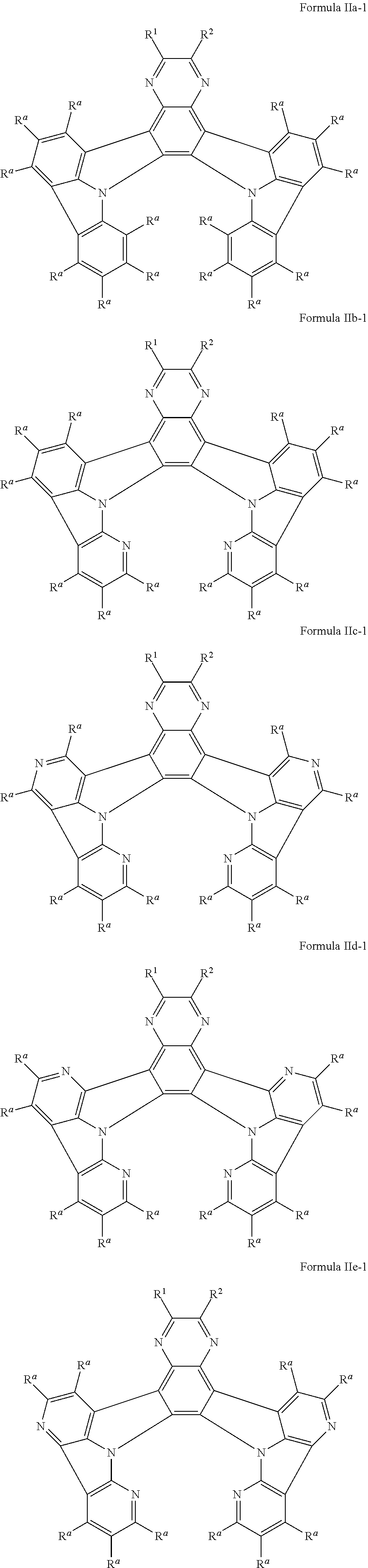

In one embodiment of the invention, at least one mono- or polycyclic, aliphatic, aromatic and/or benzo-fused ring system is formed by R^{a}, R³, R⁴ or R⁵ substituents together with one or more further R^{a}, R³, R⁴ or R⁵ substituents. Examples for such structures are shown below:

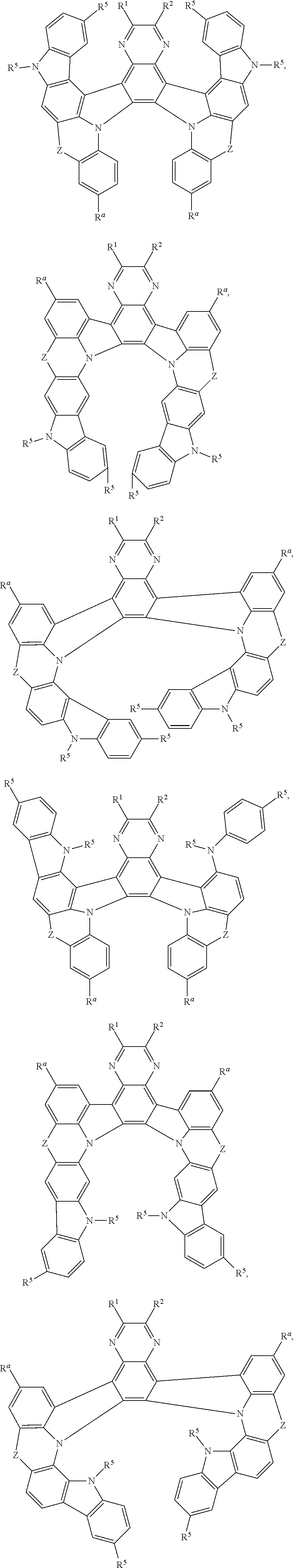

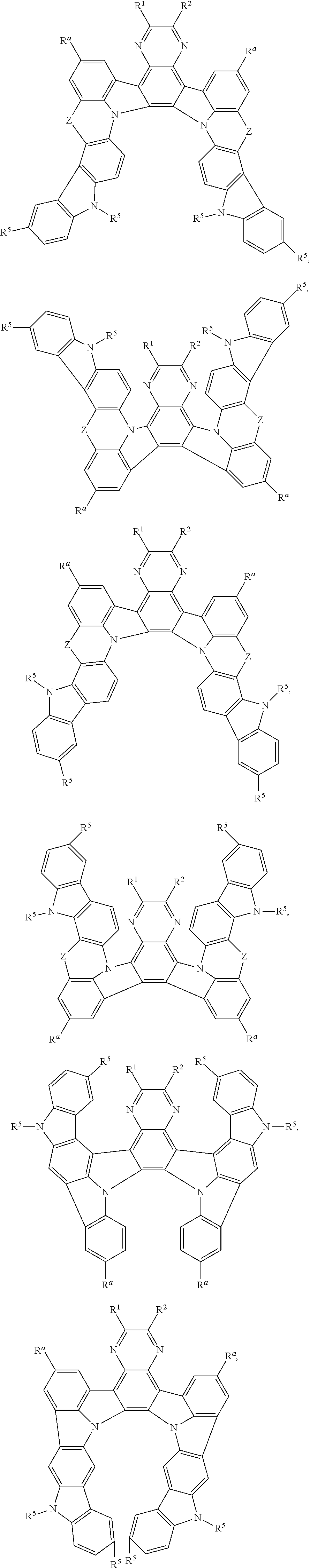

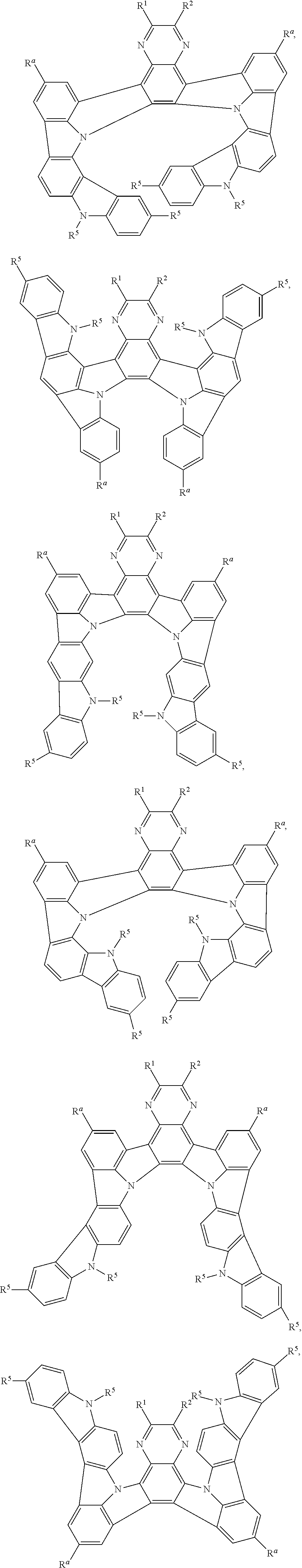

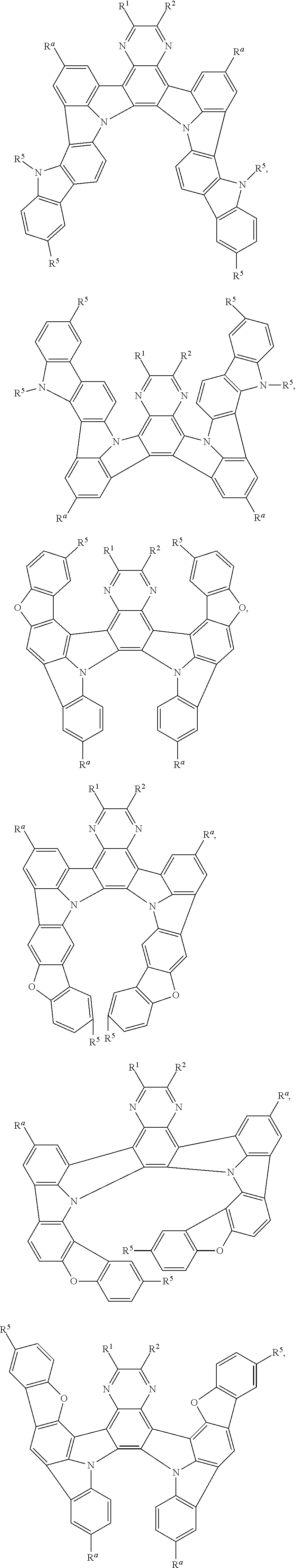

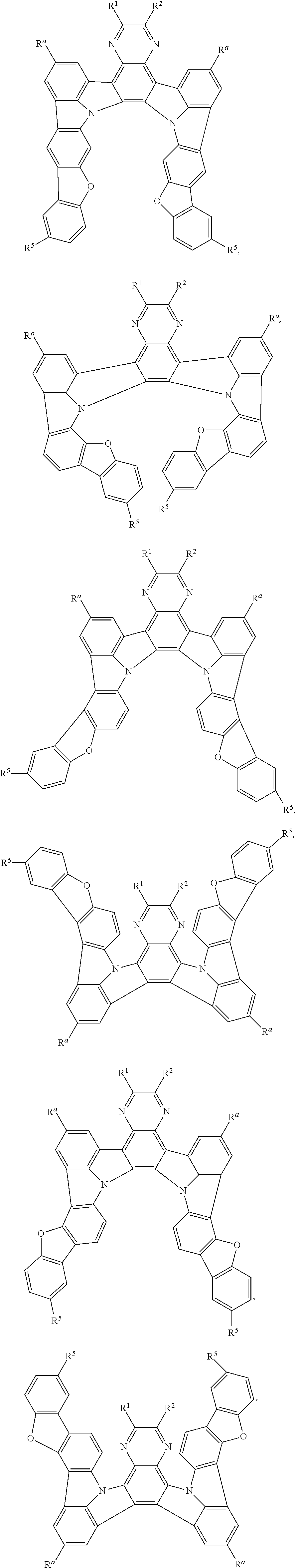

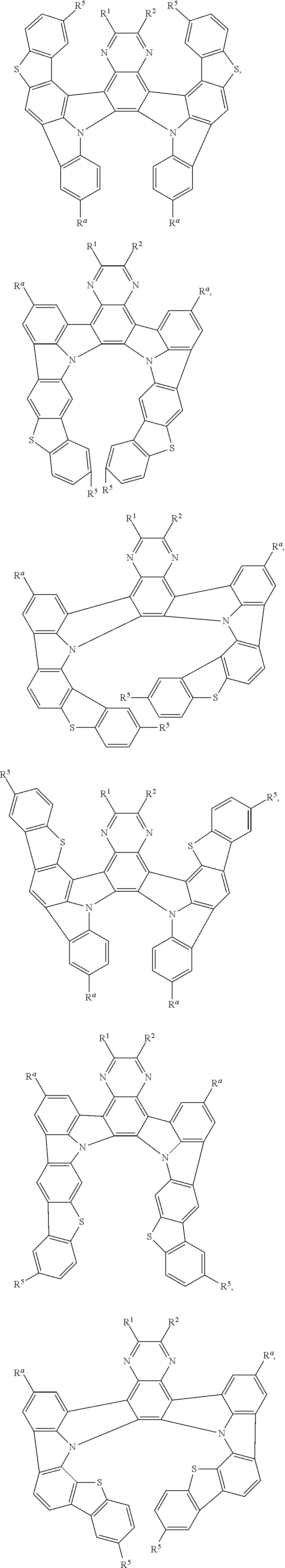

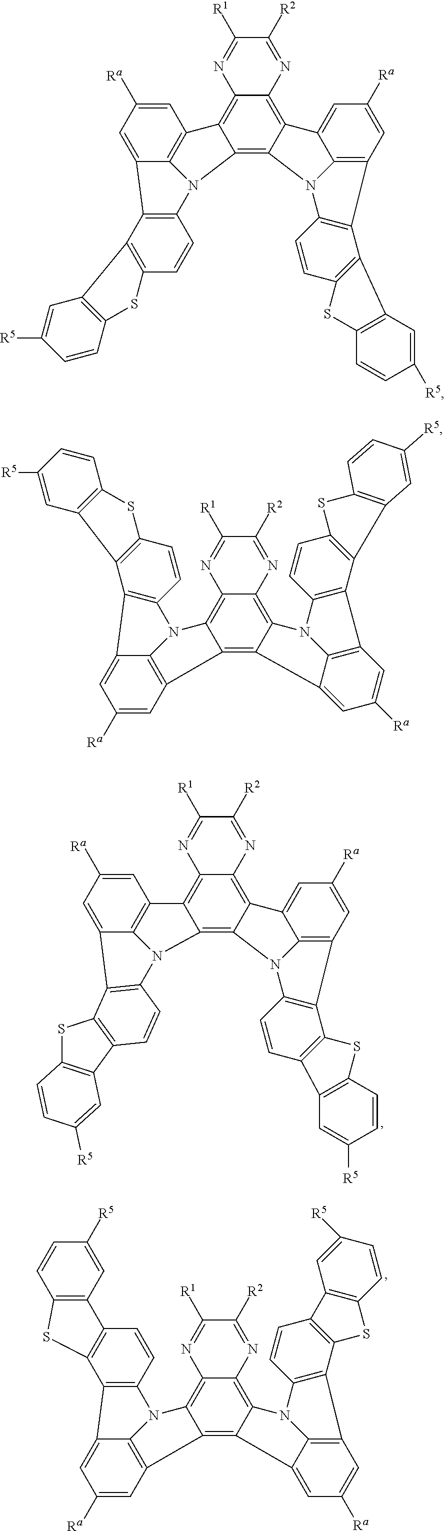

In a preferred embodiment of the invention, R¹ and R² is independently from each other selected from the group consisting of
phenyl, which is optionally substituted with one or more substituents R⁵; and
pyridine, which is optionally substituted with one or more substituents R⁵.

In further embodiments of the invention, the organic molecules include or consist of a structure selected from the group consisting of Formula IIIa, Formula IIIb, Formula IIIc, Formula IIId, Formula IIIe, Formula IIIf, Formula IIIg, Formula IIIh, and Formula IIIi:

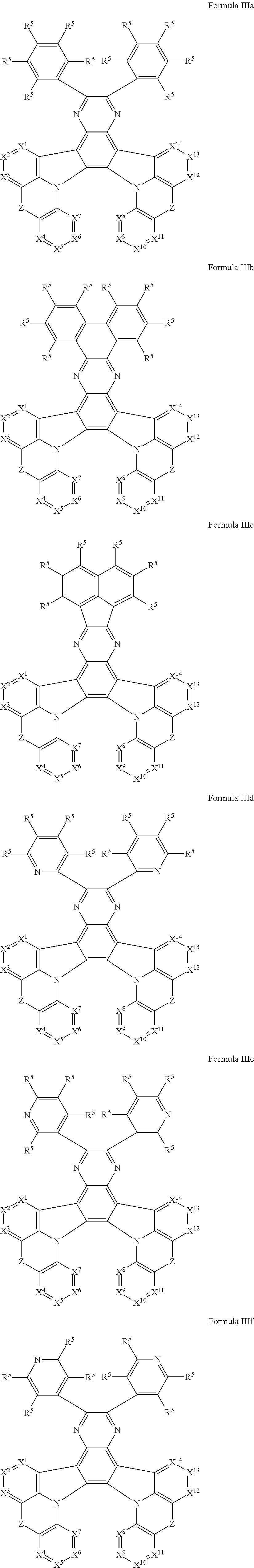

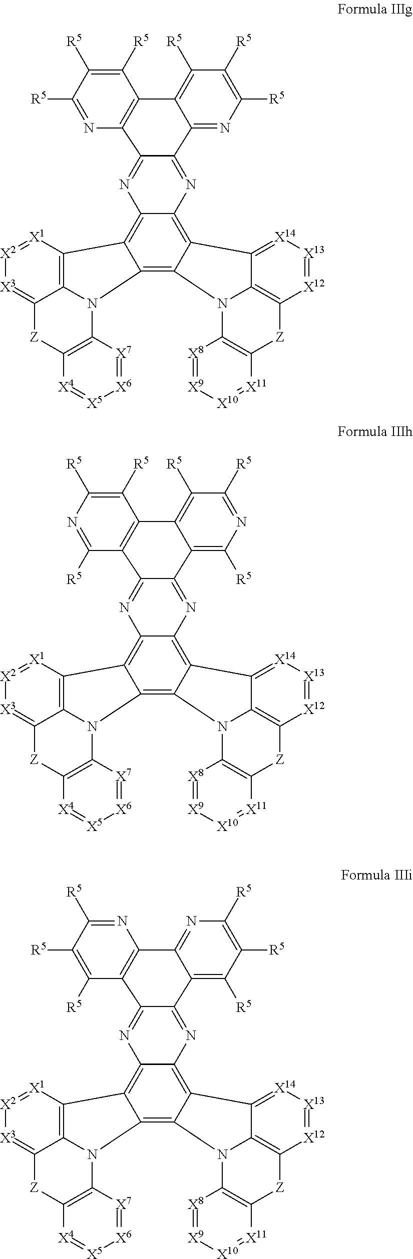

In certain embodiments of the invention, Z is a direct bond at each occurrence.

In further embodiments of the invention, the organic molecules include or consist of a structure selected from the group consisting of Formula IVa, Formula IVb, Formula IVc, Formula IVd, Formula IVe, Formula IVf, Formula IVg, Formula IVh, and Formula IVi:

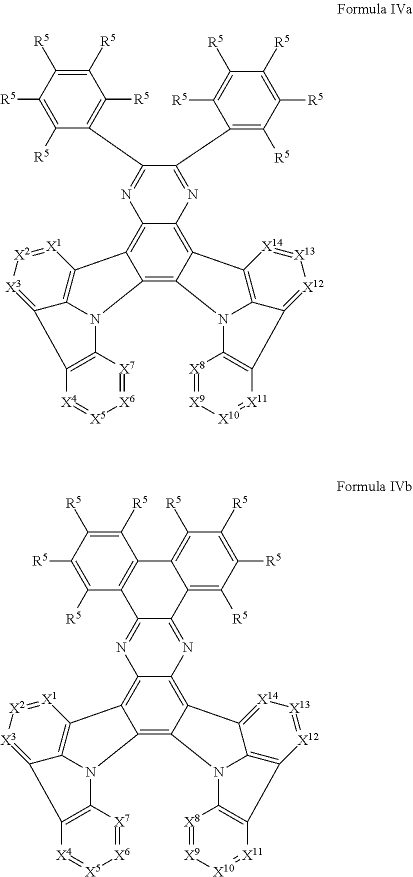

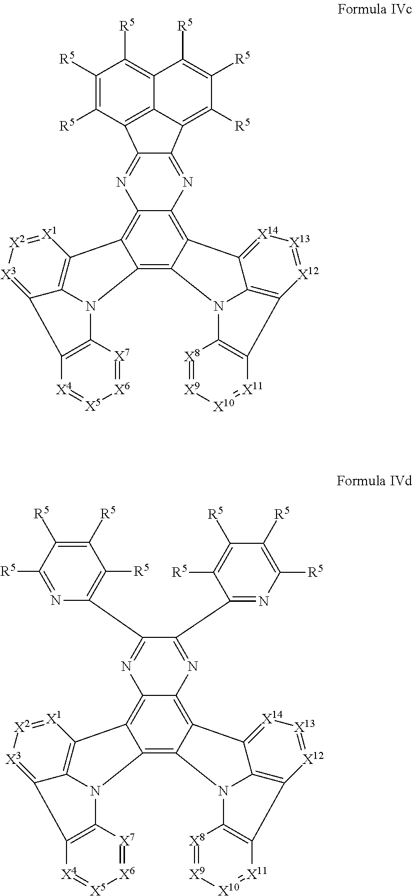

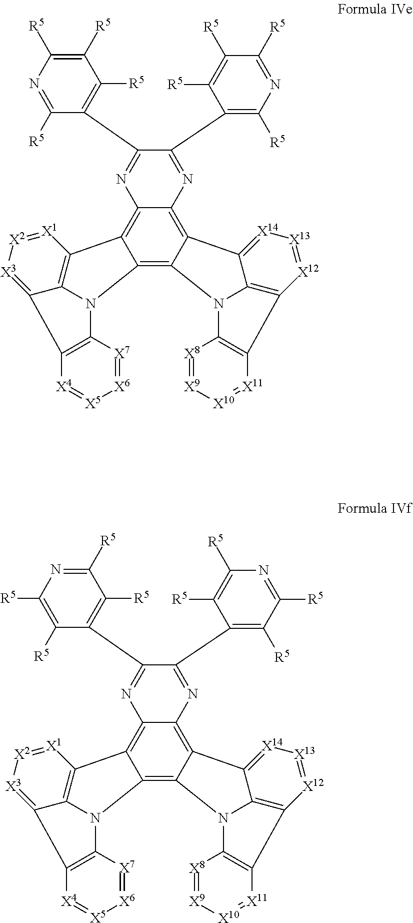

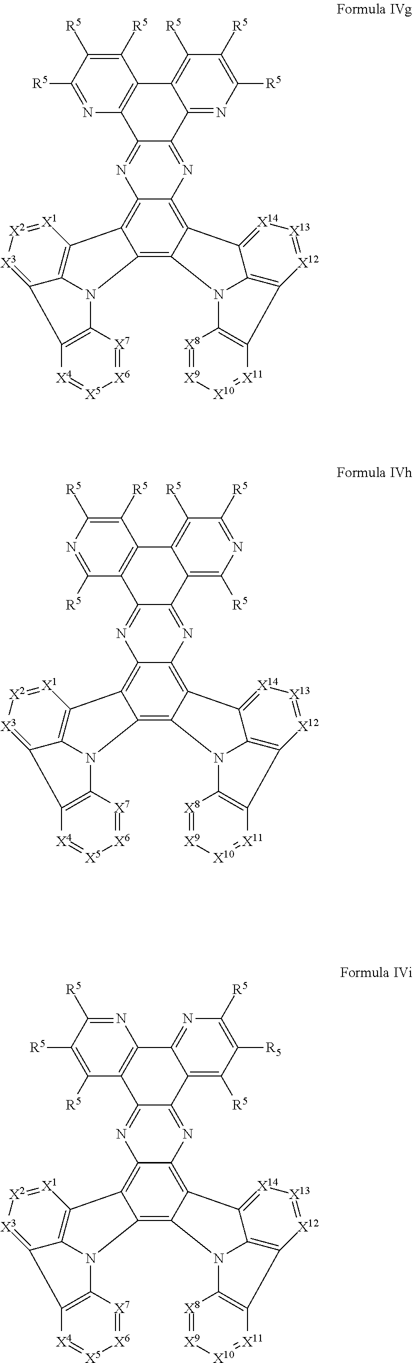

In certain embodiments of the invention, less than five atoms selected from the group consisting of X¹, X², X³, X⁴, X⁵, X⁶, X⁷, X⁸, X⁹, X¹⁰, X¹¹, X¹², X¹³, and X¹⁴ are N.

In one embodiment of the invention, exactly 2n atoms selected from the group consisting of X¹, X², X³, X⁴, X⁵, X⁶, X⁷, X⁸, X⁹, X¹⁰, X¹¹, X¹², X¹³, and X¹⁴ are N, wherein n is an integer selected from 0, 1, and 2.

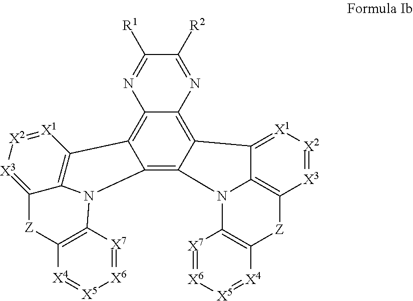

In one embodiment of the invention, the organic molecules include or consist of a structure of Formula Ib, wherein exactly n atoms of each of the two groups that are both selected from the group consisting of X¹, X², X³, X⁴, X⁵, X⁶, and X⁷ are N, wherein n is an integer selected from 0, 1, and 2.

In one embodiment, wherein the substituents R² and R¹ are the same, i.e., the substituent R² is equal to R¹; put differently, R²═R¹.

In one embodiment of the invention, the organic molecules include or consist of a structure of Formula V:

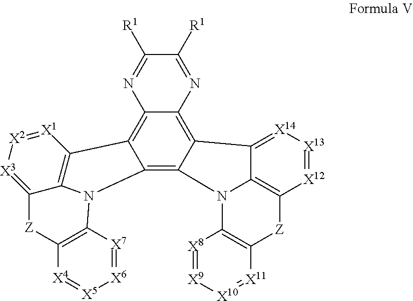

wherein the substituents R¹, R^{a}, R³, R⁴ or R⁵ independently from each other optionally form a mono- or polycyclic, aliphatic, aromatic and/or benzo-fused ring system with one or more substituents R¹, R^{a}, R³, R⁴ or R⁵.

In one embodiment of the invention, the organic molecules include or consist of a structure of Formula Va:

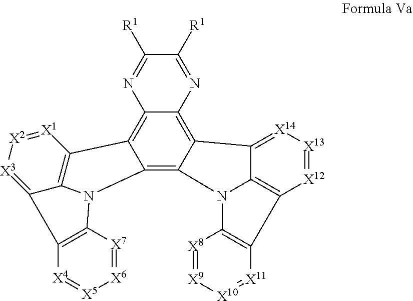

wherein the substituents R¹, R^{a}, or R⁵ independently from each other optionally form a mono- or polycyclic, aliphatic, aromatic and/or benzo-fused ring system with one or more substituents R¹, R^{a}, or R⁵.

In one embodiment of the invention, the organic molecules include or consist of a structure of formula Vb:

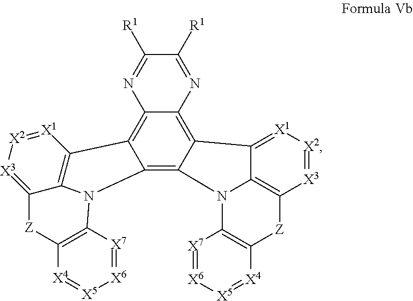

wherein
X¹, X², X³, X⁴, X⁵, and X⁶ is independently from each other selected from the group consisting of N and CR^{a}, and
the substituents R¹, R^{a}, R³, R⁴ or R⁵ independently from each other optionally form a mono- or polycyclic, aliphatic, aromatic and/or benzo-fused ring system with one or more substituents R¹, R^{a}, R³, R⁴ or R⁵.

In one embodiment of the invention, the organic molecules include or consist of a structure selected from the group consisting of Formula Vb-1, Formula Vb-2, Formula Vb-3, Formula Vb-4, Formula Vb-5, Formula Vb-6, Formula Vb-7, Formula Vb-8, and Formula Vb-9:

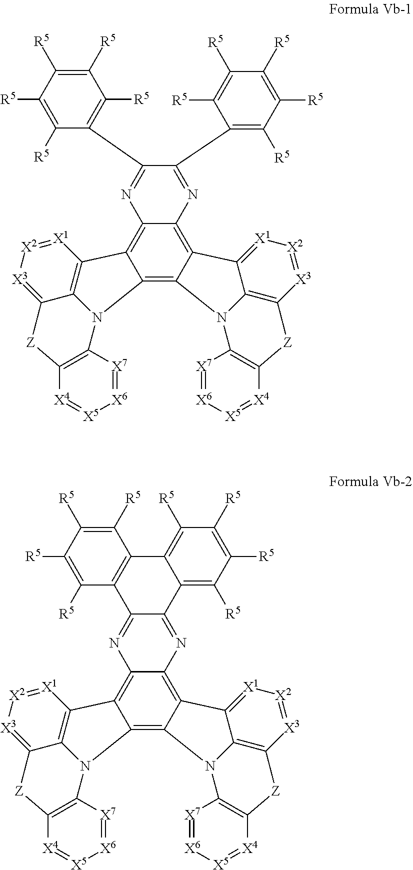

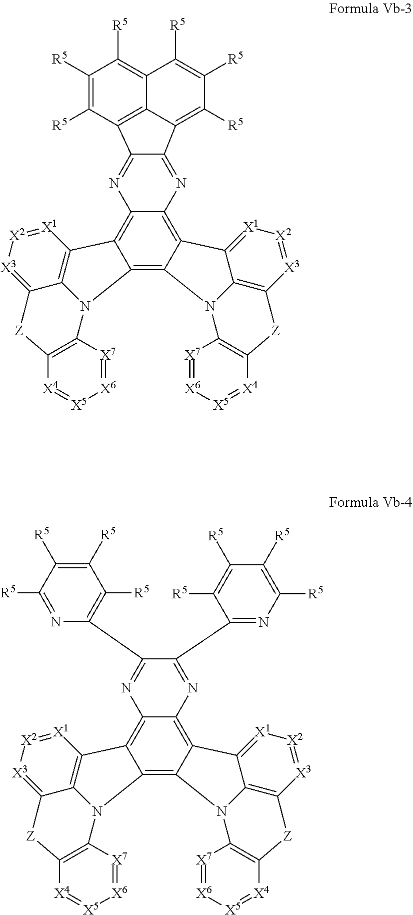

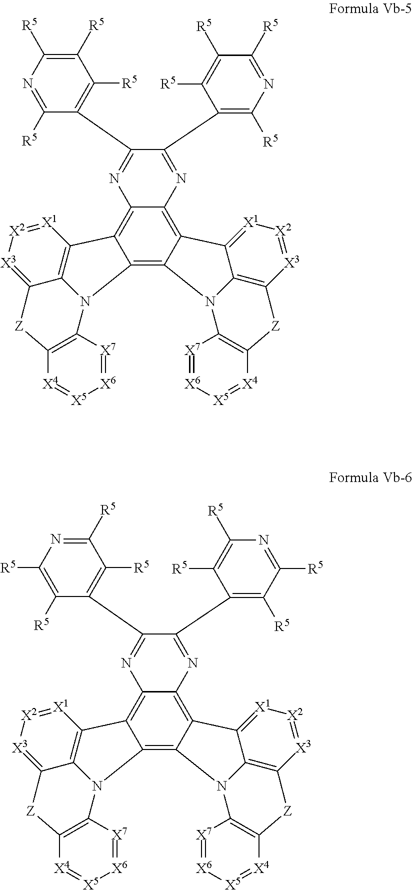

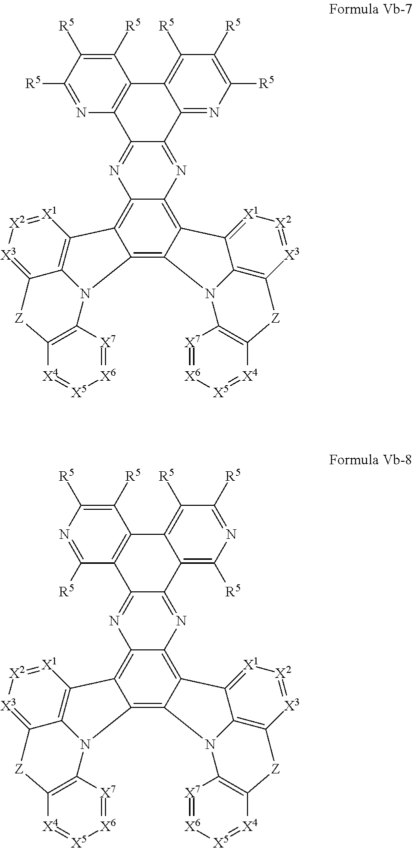

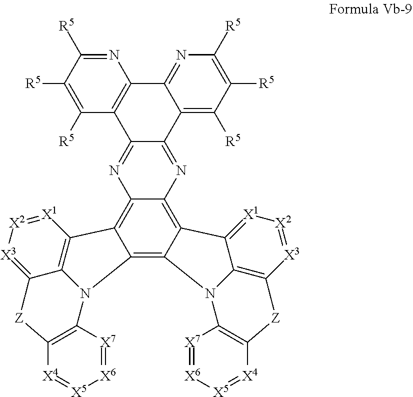

In one embodiment of the invention, the organic molecules include or consist of a structure of Formula Vc:

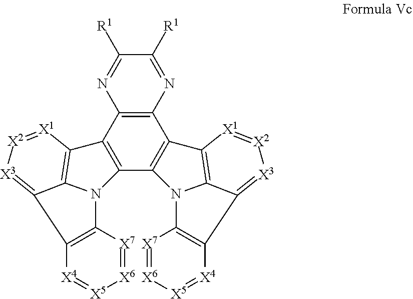

wherein
X¹, X², X³, X⁴, X⁵, X⁶ and X⁷ is independently from each other selected from the group consisting of N and CR^{a}, and
the substituents R¹, R^{a} or R⁵ independently from each other optionally form a mono- or polycyclic, aliphatic, aromatic and/or benzo-fused ring system with one or more substituents R¹, R^{a} or R⁵.

In one embodiment of the invention, the organic molecules include or consist of a structure selected from the group consisting of Formula Vc-1, Formula Vc-2, Formula Vc-3, Formula Vc-4, Formula Vc-5, Formula Vc-6, Formula Vc-7, Formula Vc-8, and Formula Vc-9:

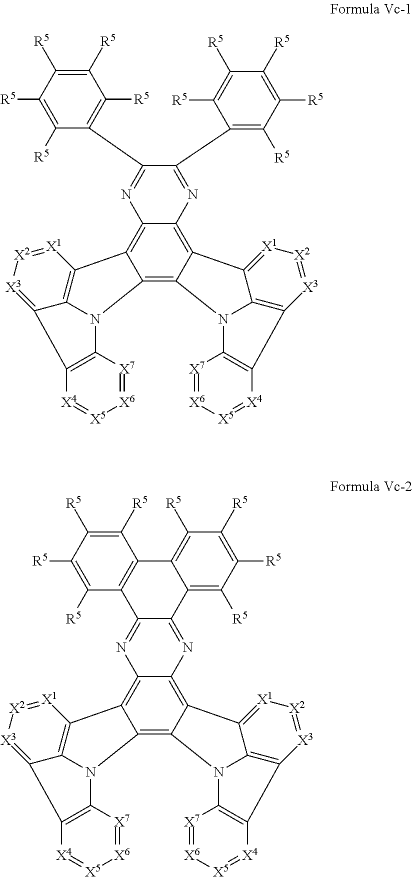

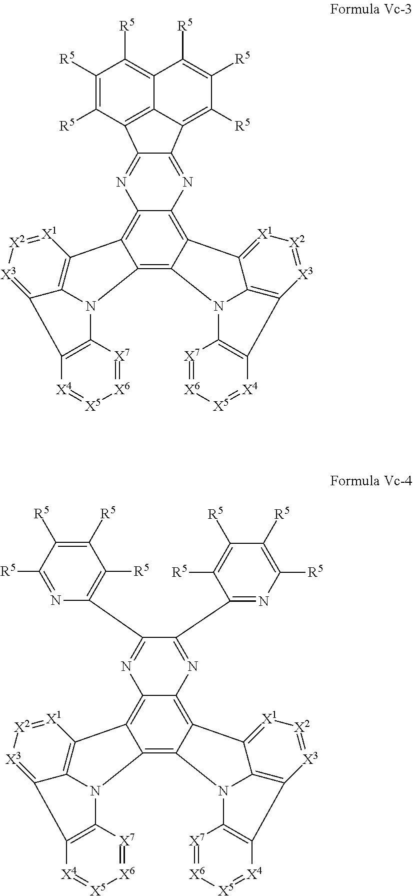

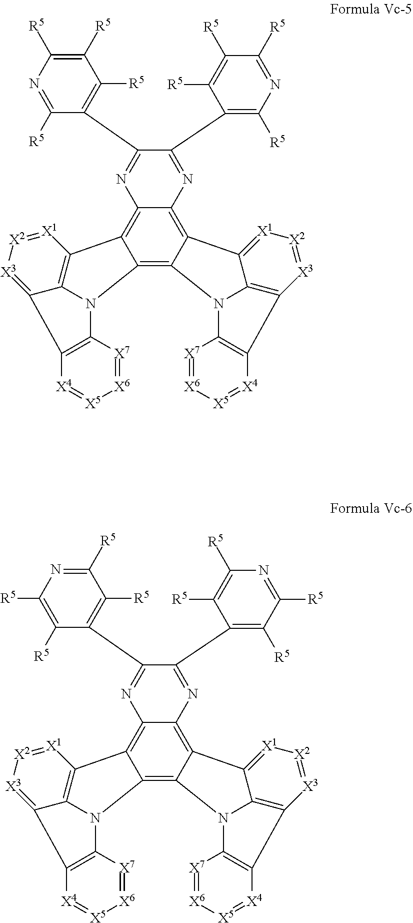

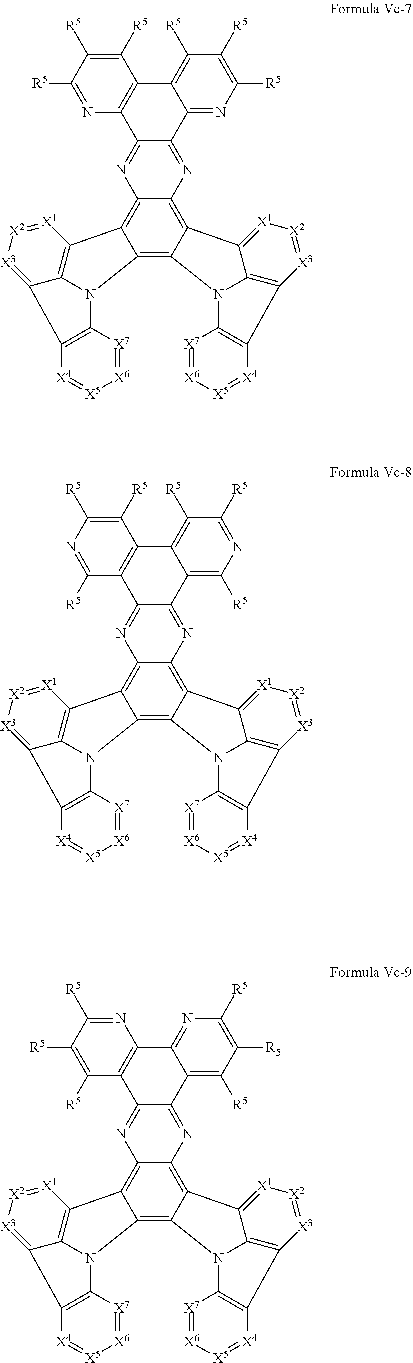

In certain embodiments of the invention, R⁵ is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

In certain embodiments of the invention, R⁵ is at each occurrence independently from one another selected from the group consisting of:
hydrogen,
C₁-C₄₀-alkyl, which is optionally substituted with one or more substituents R⁶ and
C₆-C₆₀-aryl, which is optionally substituted with one or more substituents R⁶.

In certain embodiments of the invention, R⁵ is at each occurrence independently from one another selected from the group consisting of:
hydrogen,
C₁-C₄-alkyl, which is optionally substituted with one or more substituents R⁶, and
phenyl, which is optionally substituted with one or more substituents R⁶.

In certain embodiments of the invention, R⁵ is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph.

In a further embodiment of the invention, R^{a} is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

In a further embodiment of the invention, R^{a} is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph.

In one embodiment of the invention, the organic molecules include or consist of a structure of Formula VI:

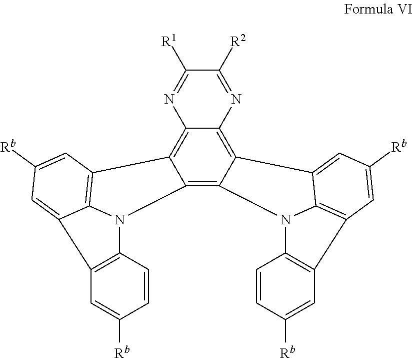

wherein the substituents R¹, R², or R⁵ independently from each other optionally form a mono- or polycyclic, aliphatic, aromatic and/or benzo-fused ring system with one or more substituents R¹, R², or R⁵, and
wherein R^{b} is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph, and
N(Ph)₂.

In one embodiment of the invention, the organic molecules include or consist of a structure of Formula VIa:

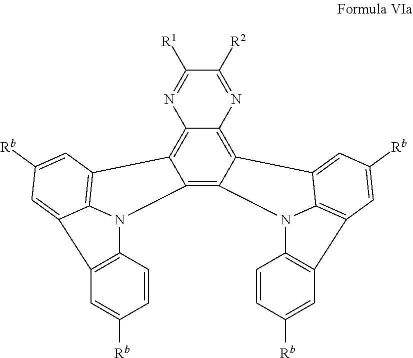

wherein the substituents R¹ or R⁵ independently from each other optionally form a mono- or polycyclic, aliphatic, aromatic and/or benzo-fused ring system with one or more substituents R¹ or R⁵.

In one embodiment of the invention, the organic molecules include or consist of a structure selected from the group consisting of Formula VIa-1, Formula VIa-2, Formula VIa-3, Formula VIa-4, Formula VIa-5, Formula VIa-6, Formula VIa-7, Formula VIa-8, and Formula VIa-9:

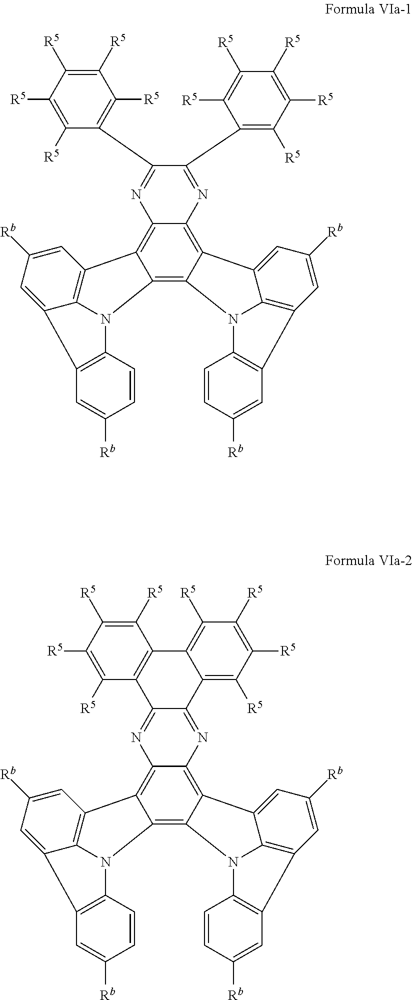

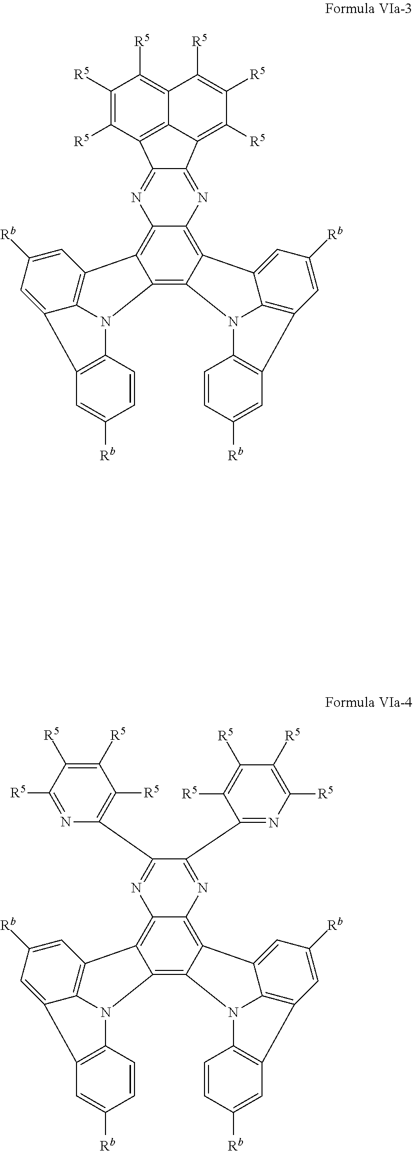

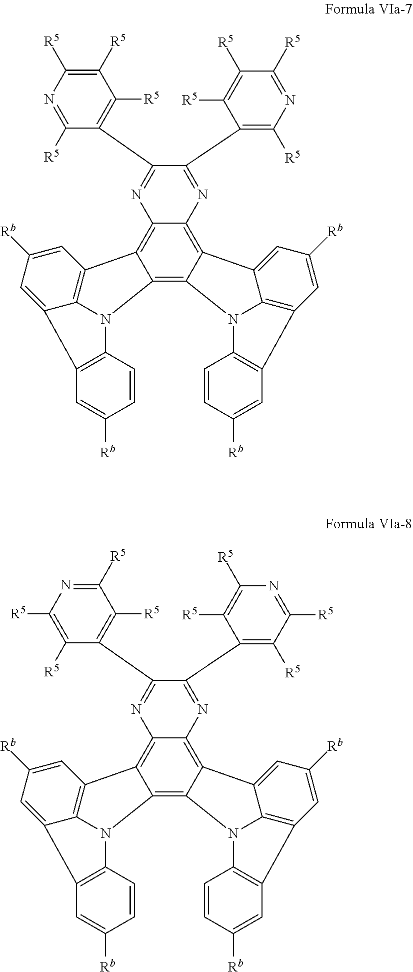

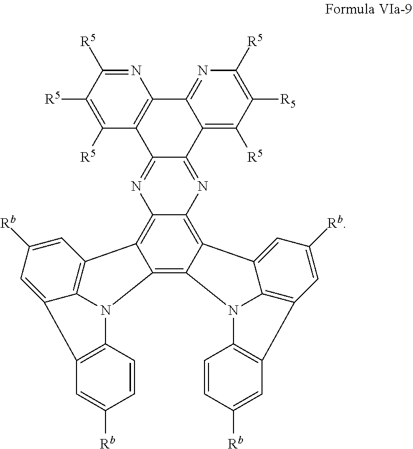

In one embodiment of the invention, the organic molecules include or consist of a structure of Formula VIb:

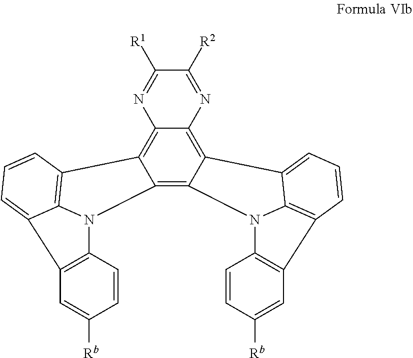

wherein the substituents R¹, R², or R⁵ independently from each other optionally form a mono- or polycyclic, aliphatic, aromatic and/or benzo-fused ring system with one or more substituents R¹, R², or R⁵, and
wherein R^{b} is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph, and
N(Ph)₂.

In one embodiment of the invention, the organic molecules include or consist of a structure of Formula VIb-1:

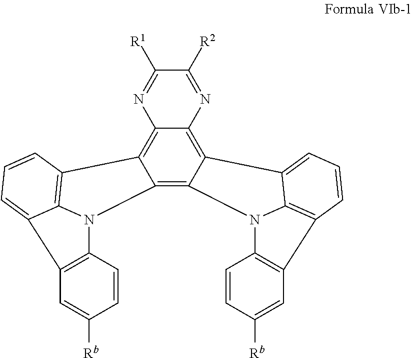

wherein the substituents R¹ or R⁵ independently from each other optionally form a mono- or polycyclic, aliphatic, aromatic and/or benzo-fused ring system with one or more substituents R¹ or R⁵.

In one embodiment of the invention, the organic molecules include or consist of a structure of Formula VIc:

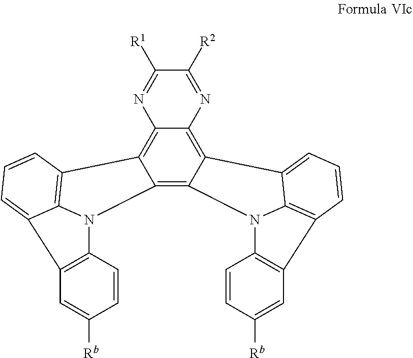

wherein the substituents R¹, R², or R⁵ independently from each other optionally form a mono- or polycyclic, aliphatic, aromatic and/or benzo-fused ring system with one or more substituents R¹, R², or R⁵, and
wherein R^{b} is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph, and
N(Ph)₂.

In one embodiment of the invention, the organic molecules include or consist of a structure of Formula VIc-1:

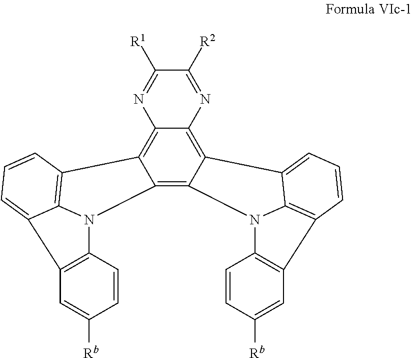

wherein the substituents R¹ or R⁵ independently from each other optionally form a mono- or polycyclic, aliphatic, aromatic and/or benzo-fused ring system with one or more substituents R¹ or R⁵.

In one embodiment of the invention, the organic molecules include or consist of a structure of Formula VII:

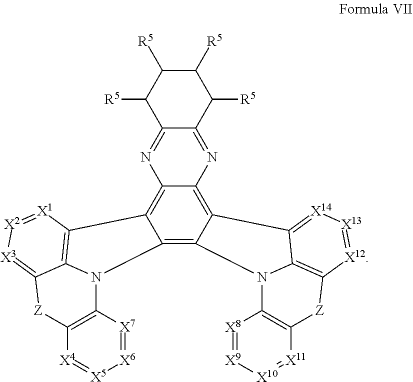

As used throughout the present application, the terms “aryl” and “aromatic” may be understood in the broadest sense as any mono-, bi- or polycyclic aromatic moieties. Accordingly, an aryl group contains 6 to 60 aromatic ring atoms, and a heteroaryl group contains 5 to 60 aromatic ring atoms, of which at least one is a heteroatom. Notwithstanding, throughout the application the number of aromatic ring atoms may be given as subscripted number in the definition of certain substituents. In particular, the heteroaromatic ring includes one to three heteroatoms. Again, the terms “heteroaryl” and “heteroaromatic” may be understood in the broadest sense as any mono-, bi- or polycyclic hetero-aromatic moieties that include at least one heteroatom. The heteroatoms may at each occurrence be the same or different and be individually selected from the group consisting of N, O and S. Accordingly, the term “arylene” refers to a divalent substituent that bears two binding sites to other molecular structures and thereby serving as a linker structure. In case, a group in the exemplary embodiments is defined differently from the definitions given here, for example, the number of aromatic ring atoms or number of heteroatoms differs from the given definition, the definition in the exemplary embodiments is to be applied. According to the invention, a condensed (annulated) aromatic or heteroaromatic polycycle is built of two or more single aromatic or heteroaromatic cycles, which formed the polycycle via a condensation reaction.

In particular, as used here throughout, the term “aryl group or heteroaryl group” includes groups which can be bound via any position of the aromatic or heteroaromatic group, derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene; pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthoimidazole, phenanthroimidazole, pyridoimidazole, pyrazinoimidazole, quinoxalinoimidazole, oxazole, benzoxazole, naphthooxazole, anthroxazole, phenanthroxazole, isoxazole, 1,2-thiazole, 1,3-thiazole, benzothiazole, pyridazine, benzopyridazine, pyrimidine, benzopyrimidine, 1,3,5-triazine, quinoxaline, pyrazine, phenazine, naphthyridine, carboline, benzocarboline, phenanthroline, 1,2,3-triazole, 1,2,4-triazole, benzotriazole, 1,2,3-oxadiazole, 1,2,4-oxadiazole, 1,2,5-oxadiazole, 1,2,3,4-tetrazine, purine, pteridine, indolizine and benzothiadiazole or combinations of the abovementioned groups.

As used here throughout, the term “cyclic group” may be understood in the broadest sense as any mono-, bi- or polycyclic moieties.

As used here throughout, the term “biphenyl” as a substituent may be understood in the broadest sense as ortho-biphenyl, meta-biphenyl, or para-biphenyl, wherein ortho, meta and para are defined in regard to the binding site to another chemical moiety.

As used here throughout, the term “terphenyl” as a substituent may be understood in the broadest sense as 3-ortho-terphenyl, 4-ortho-terphenyl, 4-meta-terphenyl, 5-meta-terphenyl, 2-para-terphenyl or 3-para-terphenyl, wherein ortho, meta and para are defined in regard to the position of the Ph moieties to each other and “2-”, “3-”, “4-” and “5-” are defined in regard to the binding site to another chemical moiety, i.e.,

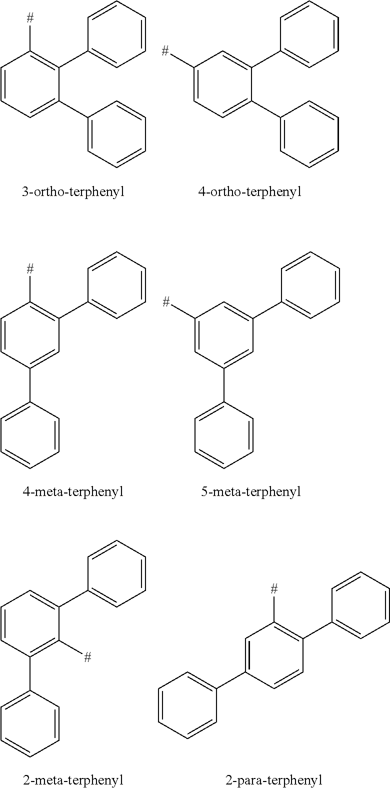

wherein # indicates the binding site to another chemical moiety.

As used here throughout, the term “naphthyl” as a naphthalene substituent may be understood in the broadest sense as 1-naphthyl and 2-naphthyl, wherein “1-” and “2-” are defined in regard to the binding site to another chemical moiety, i.e.,

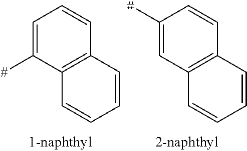

wherein # indicates the binding site to another chemical moiety.

As used here throughout, the term “anthracene” as a substituent may be understood in the broadest sense as 1-anthracenyl, 2-anthracenyl and 9-anthracenyl wherein “1-”, “2-” and “9-” are defined in regard to the binding site to another chemical moiety, i.e.,

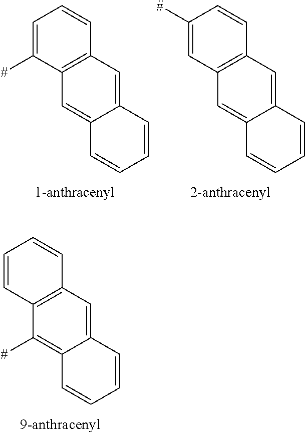

wherein # indicates the binding site to another chemical moiety.

As used here throughout, the term “alkyl group” may be understood in the broadest sense as any linear, branched, or cyclic alkyl substituent. In particular, the term alkyl includes the substituents methyl (Me), ethyl (Et), n-propyl (ⁿPr), i-propyl (ⁱPr), cyclopropyl, n-butyl (ⁿBu), i-butyl (ⁱBu), s-butyl (^{s}Bu), t-butyl (^{t}Bu), cyclobutyl, 2-methylbutyl, n-pentyl, s-pentyl, t-pentyl, 2-pentyl, neo-pentyl, cyclopentyl, n-hexyl, s-hexyl, t-hexyl, 2-hexyl, 3-hexyl, neo-hexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2,2,2]octyl, 2-bicyclo[2,2,2]-octyl, 2-(2,6-dimethyl)octyl, 3-(3,7-dimethyl)octyl, adamantyl, 2,2,2-trifluoroethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)-cyclohex-1-yl, 1-(n-butyl)-cyclohex-1-yl, 1-(n-hexyl)-cyclohex-1-yl, 1-(n-octyl)-cyclohex-1-yl and 1-(n-decyl)-cyclohex-1-yl.

As used throughout, the term “alkenyl” includes linear, branched, and cyclic alkenyl substituents. The term “alkenyl group”, for example, includes the substituents ethenyl, propenyl, butenyl, pentenyl, cyclopentenyl, hexenyl, cyclohexenyl, heptenyl, cycloheptenyl, octenyl, cyclooctenyl or cyclooctadienyl.

As used throughout, the term “alkynyl” includes linear, branched, and cyclic alkynyl substituents. The term “alkynyl group”, for example, includes ethynyl, propynyl, butynyl, pentynyl, hexynyl, heptynyl or octynyl.

As used throughout, the term “alkoxy” includes linear, branched, and cyclic alkoxy substituents. The term “alkoxy group” exemplarily includes methoxy, ethoxy, n-propoxy, i-propoxy, n-butoxy, i-butoxy, s-butoxy, t-butoxy and 2-methylbutoxy.

As used throughout, the term “thioalkoxy” includes linear, branched, and cyclic thioalkoxy substituents, in which the O of the exemplarily alkoxy groups is replaced by S.

As used throughout, the terms “halogen” and “halo” may be understood in the broadest sense as being preferably fluorine, chlorine, bromine, or iodine.

Whenever hydrogen (H) is mentioned herein, it could also be replaced by deuterium at each occurrence.

It is understood that when a molecular fragment is described as being a substituent or otherwise attached to another moiety, its name may be written as if it were a fragment (e.g., naphthyl, dibenzofuryl) or as if it were the whole molecule (e.g., naphthalene, dibenzofuran). As used herein, these different ways of designating a substituent or attached fragment are considered to be equivalent.

In one embodiment of the invention, the organic molecules according to the invention have an emission peak in the visible or nearest ultraviolet range, i.e., in the range of a wavelength of from 380 nm to 800 nm, with a full width at half maximum of less than 0.35 eV, preferably less than 0.30 eV, more preferably less than 0.26 eV, even more preferably less than 0.22 eV or even less than 0.18 eV with 0.001 mg/mL in an organic solvent, preferably in DCM or toluene, of organic molecule at room temperature.

The energy of the first excited triplet state T1 is determined from the onset of the emission spectrum at low temperature, typically at 77 K. The phosphorescence is usually visible in a steady-state spectrum in a film of 2% by weight emitter and 98% by weight PMMA. The triplet energy can thus be determined as the onset of the phosphorescence spectrum. For Fluorescent emitter molecules, the energy of the first excited triplet state T1 is determined from the onset of the delayed emission spectrum at 77 K.

The onset of an emission spectrum is determined by computing the intersection of the tangent to the emission spectrum with the x-axis. The tangent to the emission spectrum is set at the high-energy side of the emission band and at the point at half maximum of the maximum intensity of the emission spectrum.

In one embodiment, the organic molecules according to the invention have an onset of the emission spectrum, which is energetically close to the emission maximum, i.e., the energy difference between the onset of the emission spectrum and the energy of the emission maximum is below 0.14 eV, preferably below 0.13 eV, or even below 0.12 eV, while the full width at half maximum (FWHM) of the organic molecules is less than 0.35 eV, preferably less than 0.30 eV, more preferably less than 0.26 eV, even more preferably less than 0.22 eV or even less than 0.18 eV with 0.001 mg/mL in DCM of organic molecule at room temperature, resulting in a CIEy coordinate below 0.20, preferably below 0.18, more preferably below 0.16 or even more preferred below 0.14.

A further aspect of the invention relates to the use of an organic molecule of the invention as a luminescent emitter or as an absorber, and/or as a host material and/or as an electron transport material, and/or as a hole injection material, and/or as a hole blocking material in an optoelectronic device.

A preferred embodiment relates to the use of an organic molecule according to the invention as a luminescent emitter in an optoelectronic device.

The optoelectronic device may be understood in the broadest sense as any device based on organic materials that is suitable for emitting light in the visible or nearest ultraviolet (UV) range, i.e., in the range of a wavelength of from 380 to 800 nm. More preferably, the optoelectronic device may be able to emit light in the visible range, i.e., light of from 400 nm to 800 nm.

In the context of such use, the optoelectronic device is more particularly selected from the group consisting of:
organic light-emitting diodes (OLEDs),
light-emitting electrochemical cells,
OLED sensors, especially in gas and vapor sensors that are not hermetically shielded to the surroundings,
organic diodes,
organic solar cells,
organic transistors,
organic field-effect transistors,
organic lasers, and
down-conversion elements.

In a preferred embodiment in the context of such use, the optoelectronic device is a device selected from the group consisting of an organic light emitting diode (OLED), a light emitting electrochemical cell (LEC), and a light-emitting transistor.

In the case of the use, the fraction of the organic molecule according to the invention in the emission layer in an optoelectronic device, more particularly in an OLED, is 0.1% to 99% by weight, more particularly 1% to 80% by weight. In an alternative embodiment, the proportion of the organic molecule in the emission layer is 100% by weight.

In one embodiment, the light-emitting layer includes not only the organic molecules according to the invention, but also a host material whose triplet (T1) and singlet (S1) energy levels are energetically higher than the triplet (T1) and singlet (S1) energy levels of the organic molecule.

A further aspect of the invention relates to a composition including or consisting of:
(a) at least one organic molecule according to the invention, in particular in the form of an emitter, and
(b) one or more triplet-triplet annihilation (TTA) host materials, which differ from the organic molecule according to the invention, and
(c) optionally one or more TADF materials, and
(d) optionally one or more dyes and/or one or more solvents.

A further aspect of the invention relates to a composition including or consisting of:
(a) at least one organic molecule according to the invention, in particular in the form of an emitter, and
(b) one or more host materials, which differ from the organic molecule according to the invention, and
(c) one or more TADF materials, and
(d) optionally, one or more dyes and/or one or more solvents.

A further aspect of the invention relates to a composition including or consisting of:
(a) at least one organic molecule according to the invention, in particular in the form of an emitter, and
(b) one or more host materials, which differ from the organic molecule according to the invention, and
(c) one or more phosphorescent materials, and
(d) optionally, one or more dyes and/or one or more solvents.

In a particular embodiment, the light-emitting layer EML includes or essentially consists of a composition including or consisting of:
(i) 0.1-10% by weight, preferably 0.5-5% by weight, in particular 1-3% by weight, of one or more organic molecules according to the invention;
(ii) 5-99% by weight, preferably 15-85% by weight, in particular 20-75% by weight, of at least one host compound H; and
(iii) 0.9-94.9% by weight, preferably 14.5-80% by weight, in particular 24-77% by weight, of at least one further host compound D with a structure differing from the structure of the molecules according to the invention; and
(iv)optionally 0-94% by weight, preferably 0-65% by weight, in particular 0-50% by weight, of a solvent; and
(v) optionally 0-30% by weight, in particular 0-20% by weight, preferably 0-5% by weight, of at least one further emitter molecule F with a structure differing from the structure of the molecules according to the invention.

Compositions with One or More TTA Host Material

In a preferred embodiment, in the optoelectronic device of the present invention, the light-emitting layer B includes (or consists of):
(i) 10-84% by weight of the TTA material H^{N};
(ii) 0-30% by weight of the TADF material E^{B}; and
(iii) 0.1-10% by weight of the emitter according to the invention; and optionally
(iv) 0-74% by weight of one or more solvents.

In a preferred embodiment, the percentage numbers of (i)-(iv) sum up to 100% by weight.

In another preferred embodiment, in the optoelectronic device of the present invention, the light-emitting layer B includes (or consists of):
(i) 56-90% by weight of the TTA material H^{N};
(ii) 0-5% by weight of the TADF material E^{B}; and
(iii) 0.5-5% by weight of the emitter according to the invention; and optionally
(iv) 0-34% by weight of one or more solvents.

In a preferred embodiment, the percentage numbers of (i)-(iv) sum up to 100% by weight.

Compositions with One or More TADF Material

In one embodiment, the light-emitting layer B includes:
(i) 10-89.9% by weight of one or more p-host compound H^{P};
(ii) 0-79.9% by weight of one or more n-host compound H^{N};
(iii) 10-50% by weight of one or more TADF material E^{B}; and
(iv) 0.1-10% by weight of one or more emitter according to the invention; and
(v) 0-79.9% by weight of one or more solvents.

In one embodiment, the light-emitting layer B includes:
(i) 22-87.5% by weight of one or more p-host compound H^{P};
(ii) 0-65.5% by weight of one or more n-host compound H^{N};
(iii) 12-40% by weight of one or more TADF material E^{B}; and
(iv) 0.5-5% by weight of one or more emitter according to the invention; and
(v) 0-65.5% by weight of one or more solvents.

Compositions with One or More Phosphorescent Material

In a preferred embodiment where H^{N} is optional, in the optoelectronic device of the present invention, the light-emitting layer B includes (or consists of):
(i) 10-84.9% by weight of the host compound H^{P};
(ii) 0-84.9% by weight of the host compound H^{N};
(iii) 5-15% by weight of the Phosphorescence material E^{B}; and
(iv) 0.1-10% by weight of the emitter according to the invention; and optionally
(v) 0-84.9% by weight of one or more solvents.

In a preferred embodiment where H^{N} is optional, in the optoelectronic device of the present invention, the light-emitting layer B includes (or consists of):
(i) 22-70.5% by weight of the host compound H^{P};
(ii) 0-72.5% by weight of the host compound H^{N};
(iii) 5-10% by weight of the phosphorescence material E^{B}; and
(iv) 0.5-5% by weight of the emitter according to the invention; and optionally
(v) 0-72.5% by weight of one or more solvents.

Preferably, energy can be transferred from the host compound H to the one or more organic molecules according to the invention, in particular transferred from the first excited triplet state T1(H) of the host compound H to the first excited triplet state T1(E) of the one or more organic molecules according to the invention E and/or from the first excited singlet state S1(H) of the host compound H to the first excited singlet state S1(E) of the one or more organic molecules according to the invention E.

In one embodiment, the host compound H has a highest occupied molecular orbital HOMO(H) having an energy E^{HOMO}(H) in the range of from −5 to −6.5 eV and the at least one further host compound D has a highest occupied molecular orbital HOMO(D) having an energy E^{HOMO}(D), wherein E^{HOMO}(H)>E^{HOMO}(D).

In a further embodiment, the host compound H has a lowest unoccupied molecular orbital LUMO(H) having an energy E^{LUMO}(H) and the at least one further host compound D has a lowest unoccupied molecular orbital LUMO(D) having an energy E^{LUMO}(D), wherein E^{LUMO}(H)>E^{LUMO}(D).

In one embodiment, the host compound H has a highest occupied molecular orbital HOMO(H) having an energy E^{HOMO}(H) and a lowest unoccupied molecular orbital LUMO(H) having an energy E^{LUMO}(H), and
the at least one further host compound D has a highest occupied molecular orbital HOMO(D) having an energy E^{HOMO}(D) and a lowest unoccupied molecular orbital LUMO(D) having an energy E^{LUMO}(D)
the organic molecule according to the invention E has a highest occupied molecular orbital HOMO(E) having an energy E^{HOMO}(E) and a lowest unoccupied molecular orbital LUMO(E) having an energy E^{LUMO}(E),
wherein
E^{HOMO}(H)>E^{HOMO}(D) and the difference between the energy level of the highest occupied molecular orbital HOMO(E) of the organic molecule according to the invention E (E^{HOMO}(E)) and the energy level of the highest occupied molecular orbital HOMO(H) of the host compound H (E^{HOMO}(H)) is between −0.5 eV and 0.5 eV, more preferably between −0.3 eV and 0.3 eV, even more preferably between −0.2 eV and 0.2 eV or even between −0.1 eV and 0.1 eV; and
E^{LUMO}(H)>E^{LUMO}(D) and the difference between the energy level of the lowest unoccupied molecular orbital LUMO(E) of the organic molecule according to the invention E (E^{LUMO}(E)) and the lowest unoccupied molecular orbital LUMO(D) of the at least one further host compound D (E^{LUMO}(D)) is between −0.5 eV and 0.5 eV, more preferably between −0.3 eV and 0.3 eV, even more preferably between −0.2 eV and 0.2 eV or even between −0.1 eV and 0.1 eV.

In one embodiment of the invention the host compound D and/or the host compound H is a thermally-activated delayed fluorescence (TADF)-material. TADF materials exhibit a ΔE_{ST} value, which corresponds to the energy difference between the first excited singlet state (S1) and the first excited triplet state (T1), of less than 2500 cm⁻¹. Preferably the TADF material exhibits a ΔE_{ST} value of less than 3000 cm⁻¹, more preferably less than 1500 cm⁻¹, even more preferably less than 1000 cm⁻¹ or even less than 500 cm⁻¹.

In one embodiment, the host compound D is a TADF material and the host compound H exhibits a ΔE_{ST} value of more than 2500 cm⁻¹. In a particular embodiment, the host compound D is a TADF material and the host compound H is selected from group consisting of CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole.

In one embodiment, the host compound H is a TADF material and the host compound D exhibits a ΔE_{ST} value of more than 2500 cm⁻¹. In a particular embodiment, the host compound H is a TADF material and the host compound D is selected from group consisting of T2T (2,4,6-tris(biphenyl-3-yl)-1,3,5-triazine), T3T (2,4,6-tris(triphenyl-3-yl)-1,3,5-triazine) and/or TST (2,4,6-tris(9,9′-spirobifluorene-2-yl)-1,3,5-triazine).

In a further aspect, the invention relates to an optoelectronic device including an organic molecule or a composition of the type described here, more particularly in the form of a device selected from the group consisting of organic light-emitting diode (OLED), light-emitting electrochemical cell, OLED sensor (particularly gas and vapor sensors not hermetically externally shielded), organic diode, organic solar cell, organic transistor, organic field-effect transistor, organic laser and down-conversion element.

In a preferred embodiment, the optoelectronic device is a device selected from the group consisting of an organic light emitting diode (OLED), a light emitting electrochemical cell (LEC), and a light-emitting transistor.

In one embodiment of the optoelectronic device of the invention, the organic molecule according to the invention E is used as emission material in a light-emitting layer EML.

In one embodiment of the optoelectronic device of the invention, the light-emitting layer EML consists of the composition according to the invention described here.

When the optoelectronic device is an OLED, it may, for example, have the following layer structure:
1. substrate
2. anode layer A
3. hole injection layer, HIL
4. hole transport layer, HTL
5. electron blocking layer, EBL
6. emitting layer, EML
7. hole blocking layer, HBL
8. electron transport layer, ETL
9. electron injection layer, EIL
10. cathode layer,
wherein the OLED includes each layer selected from the group of HIL, HTL, EBL, HBL, ETL, and EIL only optionally, different layers may be merged and the OLED may include more than one layer of each layer type defined above.

Furthermore, the optoelectronic device may, in one embodiment, include one or more protective layers protecting the device from damaging exposure to harmful species in the environment including, for example, moisture, vapor and/or gases.

In one embodiment of the invention, the optoelectronic device is an OLED, with the following inverted layer structure:
1. substrate
2. cathode layer
3. electron injection layer, EIL
4. electron transport layer, ETL
5. hole blocking layer, HBL
6. emitting layer, B
7. electron blocking layer, EBL
8. hole transport layer, HTL
9. hole injection layer, HIL
10. anode layer A
wherein the OLED includes each layer selected from the group of HIL, HTL, EBL, HBL, ETL, and EIL only optionally, different layers may be merged and the OLED may include more than one layer of each layer types defined above.

In one embodiment of the invention, the optoelectronic device is an OLED, which may have a stacked architecture. In this architecture, contrary to the typical arrangement in which the OLEDs are placed side by side, the individual units are stacked on top of each other. Blended light may be generated with OLEDs exhibiting a stacked architecture, in particular white light may be generated by stacking blue, green and red OLEDs. Furthermore, the OLED exhibiting a stacked architecture may include a charge generation layer (CGL), which is typically located between two OLED subunits and typically consists of an n-doped layer and a p-doped layer with the n-doped layer of one CGL being typically located closer to the anode layer.

In one embodiment of the invention, the optoelectronic device is an OLED, which includes two or more emission layers between anode and cathode. In particular, this so-called tandem OLED includes three emission layers, wherein one emission layer emits red light, one emission layer emits green light and one emission layer emits blue light, and optionally may include further layers such as charge generation layers, blocking or transporting layers between the individual emission layers. In a further embodiment, the emission layers are adjacently stacked. In a further embodiment, the tandem OLED includes a charge generation layer between each two emission layers. In addition, adjacent emission layers or emission layers separated by a charge generation layer may be merged.

The substrate may be formed by any material or composition of materials. Most frequently, glass slides are used as substrates. Alternatively, thin metal layers (e.g., copper, gold, silver, or aluminum films) or plastic films or slides may be used. This may allow for a higher degree of flexibility. The anode layer A is mostly composed of materials allowing to obtain an (essentially) transparent film. As at least one of both electrodes should be (essentially) transparent in order to allow light emission from the OLED, either the anode layer A or the cathode layer C is transparent. Preferably, the anode layer A includes a large content or even consists of transparent conductive oxides (TCOs). Such anode layer A may, for example, include indium tin oxide, aluminum zinc oxide, fluorine doped tin oxide, indium zinc oxide, PbO, SnO, zirconium oxide, molybdenum oxide, vanadium oxide, tungsten oxide, graphite, doped Si, doped Ge, doped GaAs, doped polyaniline, doped polypyrrole and/or doped polythiophene.

The anode layer A (essentially) may consist of indium tin oxide (ITO) (e.g., (In₂O₃)_{0.9}(SnO2)_{0.1}). The roughness of the anode layer A caused by the transparent conductive oxides (TCOs) may be compensated by using a hole injection layer (HIL). Further, the HIL may facilitate the injection of quasi charge carriers (i.e., holes) in that the transport of the quasi charge carriers from the TCO to the hole transport layer (HTL) is facilitated. The hole injection layer (HIL) may include poly-3,4-ethylenedioxy thiophene (PEDOT), polystyrene sulfonate (PSS), MoO₂, V₂O₅, CuPC or CuI, in particular a mixture of PEDOT and PSS. The hole injection layer (HIL) may also prevent the diffusion of metals from the anode layer A into the hole transport layer (HTL). The HIL may, for example, include PEDOT:PSS (poly-3,4-ethylenedioxy thiophene: polystyrene sulfonate), PEDOT (poly-3,4-ethylenedioxy thiophene), mMTDATA (4,4′,4″-tris[phenyl(m-tolyl)amino]triphenylamine), Spiro-TAD (2,2′,7,7′-tetrakis(n,n-diphenylamino)-9,9′-spirobifluorene), DNTPD (N1,N1′-(biphenyl-4,4′-diyl)bis(N1-phenyl-N4,N4-di-m-tolylbenzene-1,4-diamine), NPB (N,N′-bis-(1-naphthalenyl)-N,N′-bis-phenyl-(1,1′-biphenyl)-4,4′-diamine), NPNPB (N,N′-diphenyl-N,N′-di-[4-(N,N-diphenyl-amino)phenyl]benzidine), MeO-TPD (N,N,N′,N′-tetrakis(4-methoxyphenyl)benzidine), HAT-CN (1,4,5,8,9,11-hexaazatriphenylen-hexacarbonitrile) and/or Spiro-NPD (N,N′-diphenyl-N,N′-bis-(1-naphthyl)-9,9′-spirobifluorene-2,7-diamine).

Adjacent to the anode layer A or hole injection layer (HIL), a hole transport layer (HTL) is typically located. Herein, any hole transport compound may be used. For example, electron-rich heteroaromatic compounds such as triarylamines and/or carbazoles may be used as hole transport compound. The HTL may decrease the energy barrier between the anode layer A and the light-emitting layer EML. The hole transport layer (HTL) may also be an electron blocking layer (EBL). Preferably, hole transport compounds bear comparably high energy levels of their triplet states T1. For example, the hole transport layer (HTL) may include a star-shaped heterocycle such as tris(4-carbazoy-9-ylphenyl)amine (TCTA), poly-TPD (poly(4-butylphenyl-diphenyl-amine)), [alpha]-NPD (poly(4-butylphenyl-diphenyl-amine)), TAPC (4,4′-cyclohexyliden-bis[N,N-bis(4-methylphenyl)benzenamine]), 2-TNATA (4,4′,4″-tris[2-naphthyl(phenyl)amino]triphenylamine), Spiro-TAD, DNTPD, NPB, NPNPB, MeO-TPD, HAT-CN and/or TrisPcz (9,9′-diphenyl-6-(9-phenyl-9H-carbazol-3-yl)-9H,9′H-3,3′-bicarbazole). In addition, the HTL may include a p-doped layer, which may be composed of an inorganic or organic dopant in an organic hole-transporting matrix. Transition metal oxides such as vanadium oxide, molybdenum oxide or tungsten oxide may, for example, be used as inorganic dopant. Tetrafluorotetracyanoquinodimethane (F₄-TCNQ), copper-pentafluorobenzoate (Cu(I)pFBz) or transition metal complexes may, for example, be used as organic dopant.

The EBL may, for example, include mCP (1,3-bis(carbazol-9-yl)benzene), TCTA, 2-TNATA, mCBP (3,3-di(9H-carbazol-9-yl)biphenyl), tris-Pcz, CzSi (9-(4-tert-Butylphenyl)-3,6-bis(triphenylsilyl)-9H-carbazole), and/or DCB (N,N′-dicarbazolyl-1,4-dimethylbenzene).

Adjacent to the hole transport layer (HTL), the light-emitting layer EML is typically located. The light-emitting layer EML includes at least one light emitting molecule. Particularly, the EML includes at least one light emitting molecule according to the invention E. In one embodiment, the light-emitting layer includes only the organic molecules according to the invention. Typically, the EML additionally includes one or more host materials H. For example, the host material H is selected from CBP (4,4′-Bis-(N-carbazolyl)-biphenyl), mCP, mCBP, Sif87 (dibenzo[b,d]thiophen-2-yltriphenylsilane), CzSi, Sif88 (dibenzo[b,d]thiophen-2-yl)diphenylsilane), DPEPO (bis[2-(diphenylphosphino)phenyl] ether oxide), 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole, 9-[3,5-bis(2-dibenzothiophenyl)phenyl]-9H-carbazole, T2T (2,4,6-tris(biphenyl-3-yl)-1,3,5-triazine), T3T (2,4,6-tris(triphenyl-3-yl)-1,3,5-triazine) and/or TST (2,4,6-tris(9,9′-spirobifluorene-2-yl)-1,3,5-triazine). The host material H typically should be selected to exhibit first triplet (T1) and first singlet (S1) energy levels, which are energetically higher than the first triplet (T1) and first singlet (S1) energy levels of the organic molecule.

In one embodiment of the invention, the EML includes a so-called mixed-host system with at least one hole-dominant host and one electron-dominant host. In a particular embodiment, the EML includes exactly one light emitting organic molecule according to the invention and a mixed-host system including T2T as electron-dominant host and a host selected from CBP, mCP, mCBP, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole as hole-dominant host. In a further embodiment the EML includes 50-80% by weight, preferably 60-75% by weight of a host selected from CBP, mCP, mCBP, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole; 10-45% by weight, preferably 15-30% by weight of T2T and 5-40% by weight, preferably 10-30% by weight of light emitting molecule according to the invention.

Adjacent to the light-emitting layer EML, an electron transport layer (ETL) may be located. Herein, any electron transporter may be used. Exemplarily, electron-poor compounds such as, e.g., benzimidazoles, pyridines, triazoles, oxadiazoles (e.g., 1,3,4-oxadiazole), phosphine oxides and sulfone, may be used. An electron transporter may also be a star-shaped heterocycle such as 1,3,5-tri(1-phenyl-1H-benzo[d]imidazol-2-yl)phenyl (TPBi). The ETL may include NBphen (2,9-bis(naphthalen-2-yl)-4,7-diphenyl-1,10-phenanthroline), Alq₃ (aluminum-tris(8-hydroxyquinoline)), TSPO1 (diphenyl-4-triphenylsilylphenyl-phosphine oxide), BPyTP2 (2,7-di(2,2′-bipyridin-5-yl)triphenylene), Sif87 (dibenzo[b,d]thiophen-2-yltriphenylsilane), Sif88 (dibenzo[b,d]thiophen-2-yl)diphenylsilane), BmPyPhB (1,3-bis[3,5-di(pyridin-3-yl)phenyl]benzene) and/or BTB (4,4′-bis-[2-(4,6-diphenyl-1,3,5-triazinyl)]-1,1′-biphenyl). Optionally, the ETL may be doped with materials such as Liq. The electron transport layer (ETL) may also block holes or a hole blocking layer (HBL) is introduced.

The HBL may, for example, include BCP (2,9-dimethyl-4,7-diphenyl-1,10-phenanthroline=bathocuproine), BAIq (bis(8-hydroxy-2-methylquinoline)-(4-phenylphenoxy)aluminum), NBphen (2,9-bis(naphthalen-2-yl)-4,7-diphenyl-1,10-phenanthroline), Alq₃ (aluminum-tris(8-hydroxyquinoline)), TSPO1 (diphenyl-4-triphenylsilylphenyl-phosphine oxide), T2T (2,4,6-tris(biphenyl-3-yl)-1,3,5-triazine), T3T (2,4,6-tris(triphenyl-3-yl)-1,3,5-triazine), TST (2,4,6-tris(9,9′-spirobifluorene-2-yl)-1,3,5-triazine), and/or TCB/TCP (1,3,5-tris(N-carbazolyl)benzene/1,3,5-tris(carbazol)-9-yl) benzene).

Adjacent to the electron transport layer (ETL), a cathode layer C may be located. The cathode layer C may, for example, include or may consist of a metal (e.g., Al, Au, Ag, Pt, Cu, Zn, Ni, Fe, Pb, LiF, Ca, Ba, Mg, In, W, or Pd) or a metal alloy. For practical reasons, the cathode layer may also consist of (essentially) intransparent metals such as Mg, Ca or Al. Alternatively or additionally, the cathode layer C may also include graphite and or carbon nanotubes (CNTs). Alternatively, the cathode layer C may also consist of nanoscale silver wires.

An OLED may further, optionally, include a protection layer between the electron transport layer (ETL) and the cathode layer C (which may be designated as electron injection layer (EIL)). This layer may include lithium fluoride, cesium fluoride, silver, Liq (8-hydroxyquinolinolatolithium), Li₂O, BaF₂, MgO and/or NaF.

Optionally, the electron transport layer (ETL) and/or a hole blocking layer (HBL) may also include one or more host compounds H.

In order to modify the emission spectrum and/or the absorption spectrum of the light-emitting layer EML further, the light-emitting layer EML may further include one or more further emitter molecules F. Such an emitter molecule F may be any emitter molecule known in the art. Preferably such an emitter molecule F is a molecule with a structure differing from the structure of the molecules according to the invention E. The emitter molecule F may optionally be a TADF emitter. Alternatively, the emitter molecule F may optionally be a fluorescent and/or phosphorescent emitter molecule which is able to shift the emission spectrum and/or the absorption spectrum of the light-emitting layer EML. Exemplarily, the triplet and/or singlet excitons may be transferred from the organic emitter molecule according to the invention to the emitter molecule F before relaxing to the ground state S0 by emitting light typically red-shifted in comparison to the light emitted by an organic molecule. Optionally, the emitter molecule F may also provoke two-photon effects (i.e., the absorption of two photons of half the energy of the absorption maximum).

Optionally, an optoelectronic device (e.g., an OLED) may, for example, be an essentially white optoelectronic device. For example, such white optoelectronic device may include at least one (deep) blue emitter molecule and one or more emitter molecules emitting green and/or red light. Then, there may also optionally be energy transmittance between two or more molecules as described above.

As used herein, if not defined more specifically in the particular context, the designation of the colors of emitted and/or absorbed light is as follows:
violet: wavelength range of >380-420 nm;
deep blue: wavelength range of >420-480 nm;
sky blue: wavelength range of >480-500 nm;
green: wavelength range of >500-560 nm;
yellow: wavelength range of >560-580 nm;
orange: wavelength range of >580-620 nm;
red: wavelength range of >620-800 nm.

With respect to emitter molecules, such colors refer to the emission maximum. Therefore, for example, a deep blue emitter has an emission maximum in the range of from >420 to 480 nm, a sky blue emitter has an emission maximum in the range of from >480 to 500 nm, a green emitter has an emission maximum in a range of from >500 to 560 nm, a red emitter has an emission maximum in a range of from >620 to 800 nm.

A green emitter may preferably have an emission maximum between 500 and 560 nm, more preferably between 510 and 550 nm, and even more preferably between 520 and 540 nm.

A further embodiment of the present invention relates to an OLED, which emits light with CIEx and CIEy color coordinates close to the CIEx (=0.170) and CIEy (=0.797) color coordinates of the primary color green (CIEx=0.170 and CIEy=0.797) as defined by ITU-R Recommendation BT.2020 (Rec. 2020) and thus is suited for the use in Ultra High Definition (UHD) displays, e.g., UHD-TVs. In this context, the term “close to” refers to the ranges of CIEx and CIEy coordinates provided at the end of this paragraph. In commercial applications, typically top-emitting (top-electrode is transparent) devices are used, whereas test devices as used throughout the present application represent bottom-emitting devices (bottom-electrode and substrate are transparent). Accordingly, a further aspect of the present invention relates to an OLED, whose emission exhibits a CIEx color coordinate of between 0.06 and 0.34, preferably between 0.07 and 0.29, more preferably between 0.09 and 0.24 or even more preferably between 0.12 and 0.22 or even between 0.14 and 0.19 and/or a CIEy color coordinate of between 0.44 and 0.84, preferably between 0.55 and 0.83, more preferably between 0.65 and 0.82 or even more preferably between 0.70 and 0.81 or even between 0.75 and 0.8.

Accordingly, a further aspect of the present invention relates to an OLED, which exhibits an external quantum efficiency at 14500 cd/m² of more than 10%, more preferably of more than 13%, more preferably of more than 15%, even more preferably of more than 17% or even more than 20% and/or exhibits an emission maximum between 495 nm and 580 nm, preferably between 500 nm and 560 nm, more preferably between 510 nm and 550 nm, even more preferably between 515 nm and 540 nm

A deep blue emitter may preferably have an emission maximum of below 480 nm, more preferably below 470 nm, even more preferably below 465 nm or even below 460 nm. It will typically be above 420 nm, preferably above 430 nm, more preferably above 440 nm or even above 450 nm.

Accordingly, a further aspect of the present invention relates to an OLED, which exhibits an external quantum efficiency at 1000 cd/m² of more than 8%, more preferably of more than 10%, more preferably of more than 13%, even more preferably of more than 15% or even more than 20% and/or exhibits an emission maximum between 420 nm and 500 nm, preferably between 430 nm and 490 nm, more preferably between 440 nm and 480 nm, even more preferably between 450 nm and 470 nm and/or exhibits a LT80 value at 500 cd/m² of more than 100 h, preferably more than 200 h, more preferably more than 400 h, even more preferably more than 750 h or even more than 1000 h. Accordingly, a further aspect of the present invention relates to an OLED, whose emission exhibits a CIEy color coordinate of less than 0.45, preferably less than 0.30, more preferably less than 0.20 or even more preferably less than 0.15 or even less than 0.10.

A further aspect of the present invention relates to an OLED, which emits light at a distinct color point. According to the present invention, the OLED emits light with a narrow emission band (small full width at half maximum (FWHM)). In one aspect, the OLED according to the invention emits light with a FWHM of the main emission peak of less than 0.30 eV, preferably less than 0.25 eV, more preferably less than 0.20 eV, even more preferably less than 0.19 eV or even less than 0.17 eV.

A further aspect of the present invention relates to an OLED, which emits light with CIEx and CIEy color coordinates close to the CIEx (=0.131) and CIEy (=0.046) color coordinates of the primary color blue (CIEx=0.131 and CIEy=0.046) as defined by ITU-R Recommendation BT.2020 (Rec. 2020) and thus is suited for the use in Ultra High Definition (UHD) displays, e.g., UHD-TVs. Accordingly, a further aspect of the present invention relates to an OLED, whose emission exhibits a CIEx color coordinate of between 0.02 and 0.30, preferably between 0.03 and 0.25, more preferably between 0.05 and 0.20 or even more preferably between 0.08 and 0.18 or even between 0.10 and 0.15 and/or a CIEy color coordinate of between 0.00 and 0.45, preferably between 0.01 and 0.30, more preferably between 0.02 and 0.20 or even more preferably between 0.03 and 0.15 or even between 0.04 and 0.10.

In a further aspect, the invention relates to a method for producing an optoelectronic component. In this case an organic molecule of the invention is used.

The optoelectronic device, in particular the OLED according to the present invention can be fabricated by any means of vapor deposition and/or liquid processing. Accordingly, at least one layer is
prepared by means of a sublimation process,
prepared by means of an organic vapor phase deposition process,
prepared by means of a carrier gas sublimation process,
solution processed or printed.

The methods used to fabricate the optoelectronic device, in particular the OLED according to the present invention are known in the art. The different layers are individually and successively deposited on a suitable substrate by means of subsequent deposition processes. The individual layers may be deposited using the same or differing deposition methods.

Vapor deposition processes, for example, include thermal (co)evaporation, chemical vapor deposition and physical vapor deposition. For active matrix OLED display, an AMOLED backplane is used as substrate. The individual layer may be processed from solutions or dispersions employing adequate solvents. Solution deposition process, for example, include spin coating, dip coating and jet printing. Liquid processing may optionally be carried out in an inert atmosphere (e.g., in a nitrogen atmosphere) and the solvent may be completely or partially removed by means known in the state of the art.

### EXAMPLES

### General Synthesis Scheme I

wherein X¹═X¹⁴, X²═X¹³, X³═X¹², X⁴═X¹¹, X⁵═X¹⁰, X⁶═X⁹, and X⁷═X⁸:

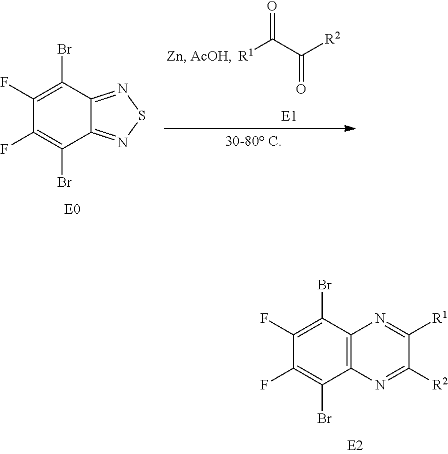

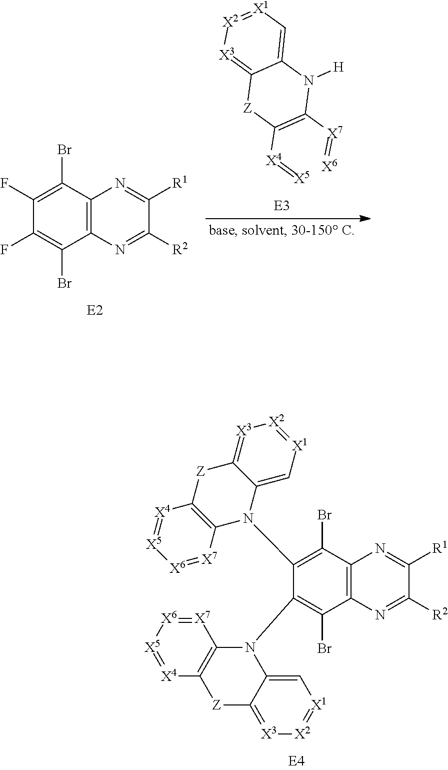

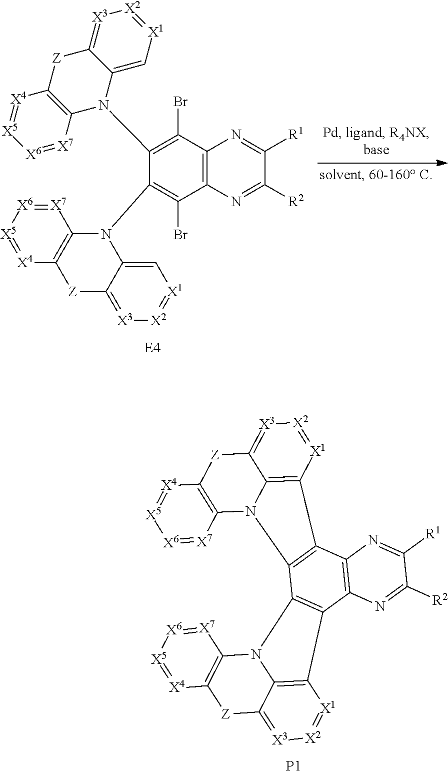

### General Procedure for Synthesis AAV1

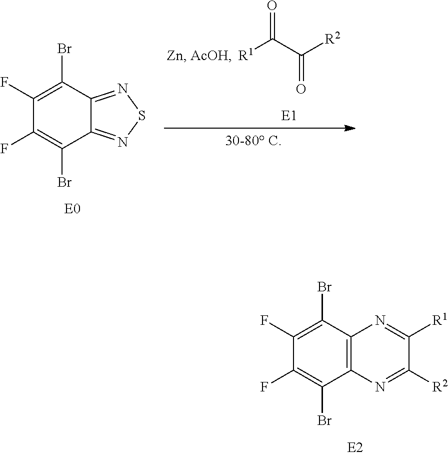

In the first step acetic acid (50 eq) added to a flask which contains 4,7-dibromo-5,6-difluorobenzo[c][1,2,5]thiadiazole (E0; 1 eq) and Zn (1.5 eq) and the mixture stirred at 60° C. Then 1,2-dione compounds E1 were added to the obtained compound and the reaction was stirred at elevated temperature (30° C. or higher) to generate the corresponding 5,8-dibromo-6,7-difluoroquinoxaline derivatives E2. Variations in the amount of compounds and temperature could be employed.

### General Procedure for Synthesis AAV2

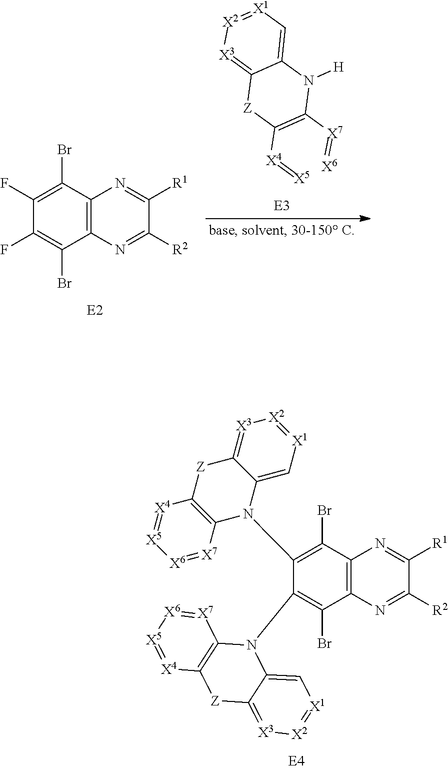

The second step was a nucleophilic aromatic substitution reaction between E2 (1 eq) and secondary amines E3 (2.3 eq) in organic solvent (for example DMSO, DMF, NMP, DMAc, DME, EtOH, MeCN, ToI, MeCN, or DCM) in the presence of base (4 eq; for example K₂CO₃, K₃PO₄, NaH, NaOtBu, KOtBu, Cs₂CO₃, KOH, NaOH, LDA, LDEA) at elevated temperature (30° C. or higher, and overnight) to obtain di-substituted compound E4. Variations in the amount of compounds, base, solvent, time, and temperature could be employed.

### General Procedure for Synthesis AAV3

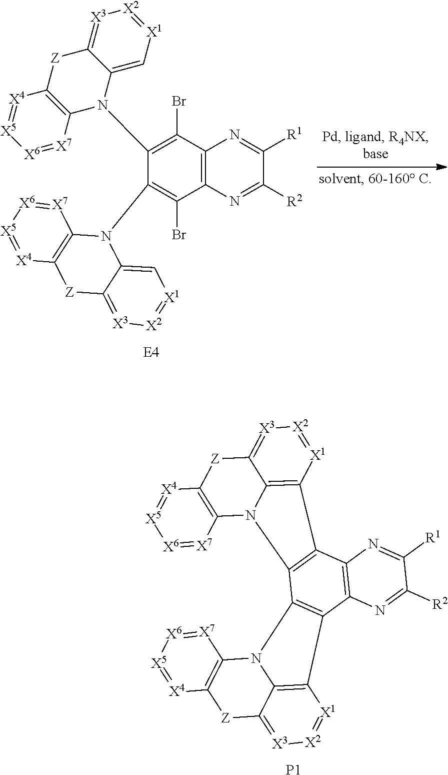

In the last step, C—H activation conducted in the presence of Pd^{II} or Pd⁰ catalyst (0.05 eq; for example Pd(OAc)₂, PdCl₂(PPh₃)₂, Pd(dppf)Cl₂, PdCl₂(PPh₃)₂, Pd(PPh₃)₄), ligand (0.08 eq; for example PPh₃, PCy₃, PCy₃—HBF₄, XPhos, S-Phos, R-Phos, xanphos, (tBu)₃P), quaternary ammonium salt (1 eq; for example TBACl, TBAB, TBAl, TBAOH, TMAB, THACl, TOACl, tetrabutylammonium tetrafluoroborate, benzyltrimethylammonium bromide), base (5 eq; for example K₂CO₃, K₃PO₄, NaH, NaOtBu, KOtBu, Cs₂CO₃, KOH, or NaOH), solvent (for example, DMAc, NMP, DMF, DME, MeCN, ToI, 1,4-dioxane, DMSO, EtOH, or THF) and at 140° C. to achieve organic molecules according to the invention, P1. Variations in the amount of compounds, phase catalyst, solvent, and temperature could be employed.

### General Synthesis Scheme II

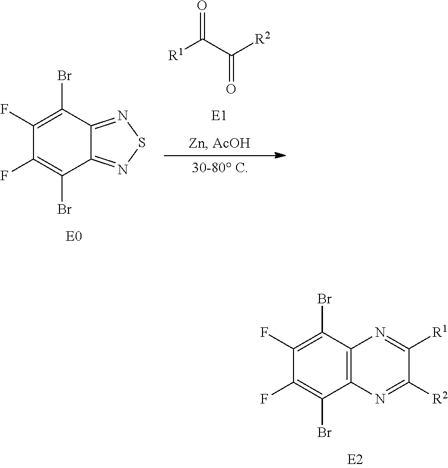

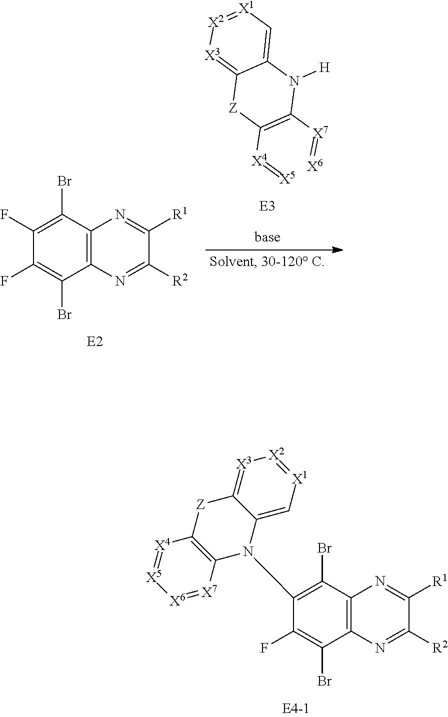

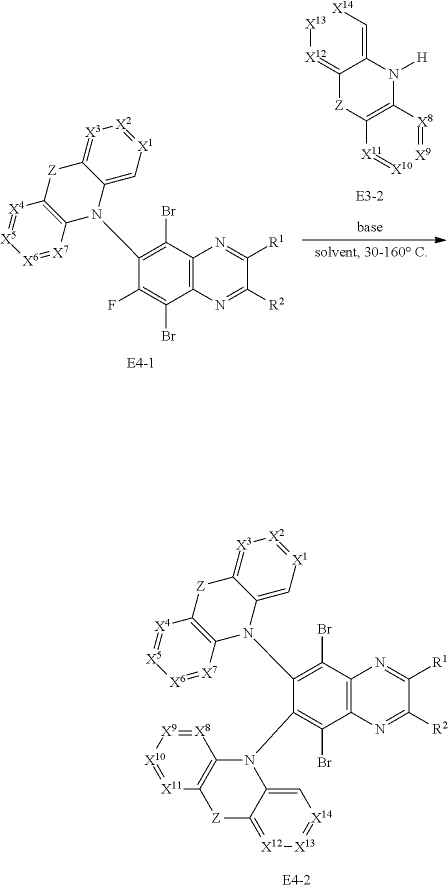

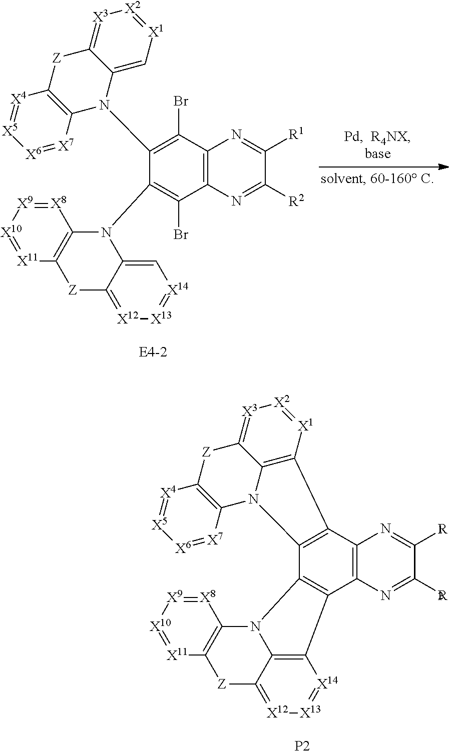

### General Procedure for Synthesis AAV2-1

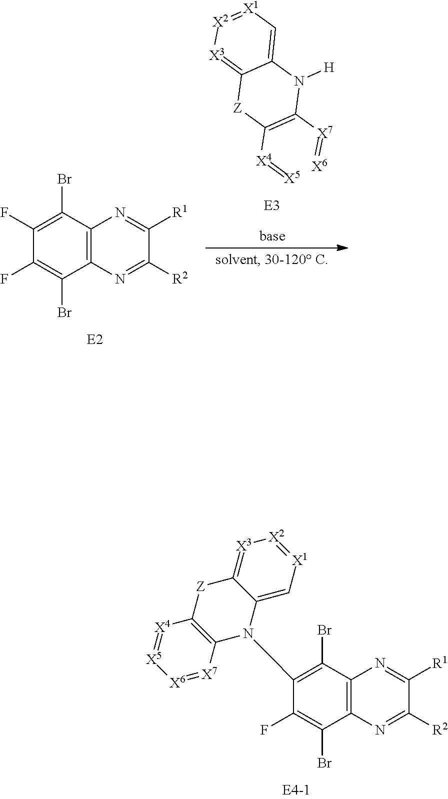

The synthesis of AAV2-1 is carried out by reacting of E2 (1.05 eq), secondary amine E3 (1 eq) in the presence of base (1.5 eq; for example K₂CO₃, K₃PO₄, NaH, NaOtBu, KOtBu, Cs₂CO₃, KOH, NaOH, LDA, or LDEA), and solvent (for example DMSO, DMF, NMP, DMAc, DME, EtOH, MeCN, ToI, MeCN, or DCM) at ambient temperature up to 120° C.

### General Procedure for Synthesis AAV2-2

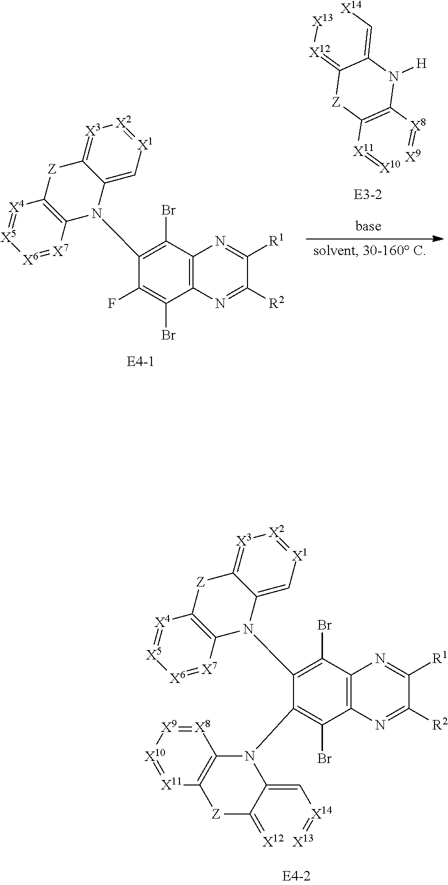

The synthesis of E4-2 is carried out from reaction of E4-1 and secondary amine E3-2. To a flask containing E4-1 (1 eq), base (1.5 eq), E3-2 (1.2 eq) was added in a solvent (for example DMSO, DMF, NMP, DMAc, DME, EtOH, MeCN, ToI, MeCN, or DCM) and the reaction mixture stirred at elevated temperature (50° C. or higher) for 12 hours. Variations in the amount of compounds, base, solvent, and temperature could be employed.

### General Procedure for Synthesis AAV2-3

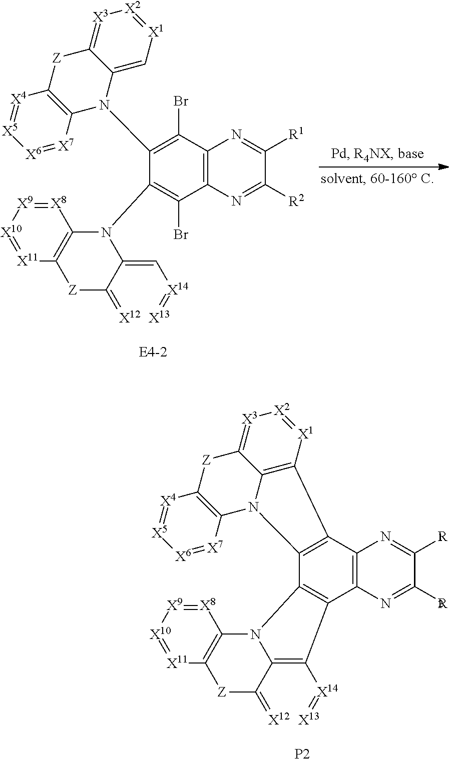

To 1 eq of E4-2, palladium catalyst (0.05 eq; for example Pd(OAc)₂, PdCl₂(PPh₃)₂, Pd(dppf)Cl₂, PdCl₂(PPh₃)₂, Pd(PPh₃)₄), ligand (0.08 eq; for example PPh₃, PCy₃, PCy₃—HBF₄, XPhos, S-Phos, R-Phos, xanphos, (tBu)₃P), quaternary ammonium salt (1 eq; for example TBACl, TBAB, TBAl, TBAOH, TMAB, THACl, TOACl, tetrabutylammonium tetrafluoroborate, benzyltrimethylammonium bromide), base (5 eq; for example K₂CO₃, K₃PO₄, NaH, NaOtBu, KOtBu, Cs₂CO₃, KOH, NaOH, LDA, LDEA) were added to a solvent (for example DMAc, NMP, DMF, DME, MeCN, ToI, 1,4-dioxane, DMSO, THF, EtOH) and at elevated temperatures (60-160° C.). Variations in the amount of compounds, phase catalyst, solvent, and temperature could be employed.

### General Synthesis Scheme III

wherein X¹═X¹⁴, X²═X¹³, X³═X¹², X⁴═X¹¹, X⁵═X¹⁰, X⁶═X⁹, and X⁷=X⁸:

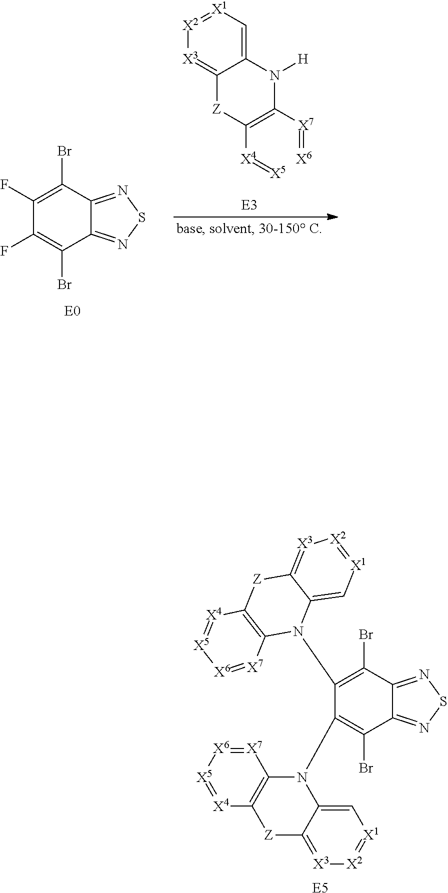

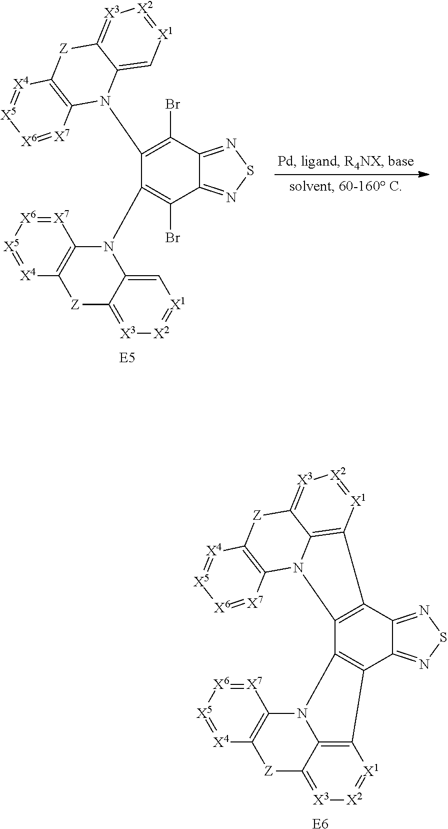

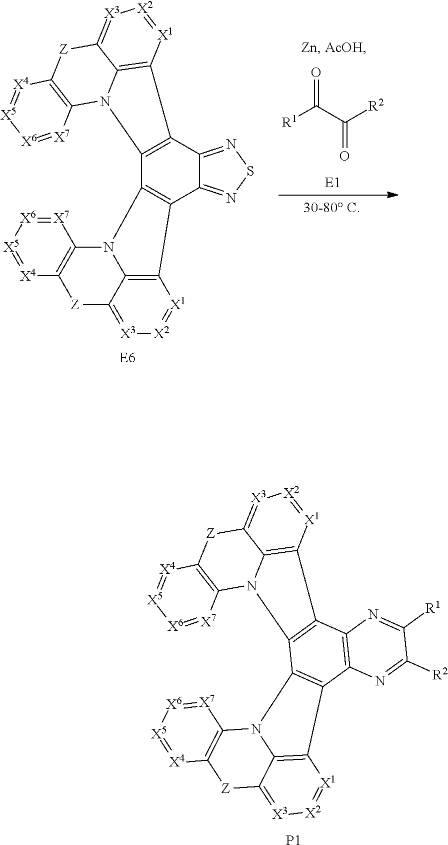

### General Procedure for Synthesis AAV3-1

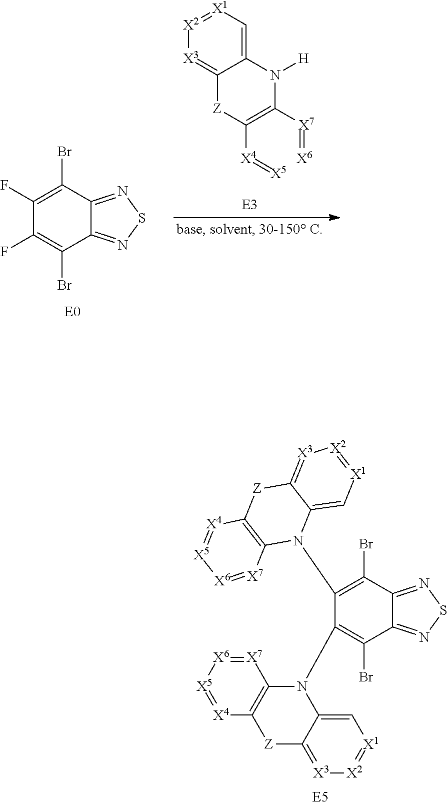

The first step was a nucleophilic aromatic substitution reaction between 4,7-dibromo-5,6-difluorobenzo[c][1,2,5]thiadiazole (E0; 1 eq) and secondary amines E3 (2.1 eq) in organic solvent (for example DMSO, DMF, NMP, DMAc, DME, EtOH, MeCN, ToI, MeCN, or DCM) in the presence of base (4 eq; for example K₂CO₃, K₃PO₄, NaH, NaOtBu, KOtBu, Cs₂CO₃, KOH, NaOH, LDA, LDEA) at elevated temperature (30° C. or higher, and overnight) to obtain di-substituted compound E5. Variations in the amount of compounds, base, solvent, time, and temperature may be employed.

### General Procedure for Synthesis AAV3-2

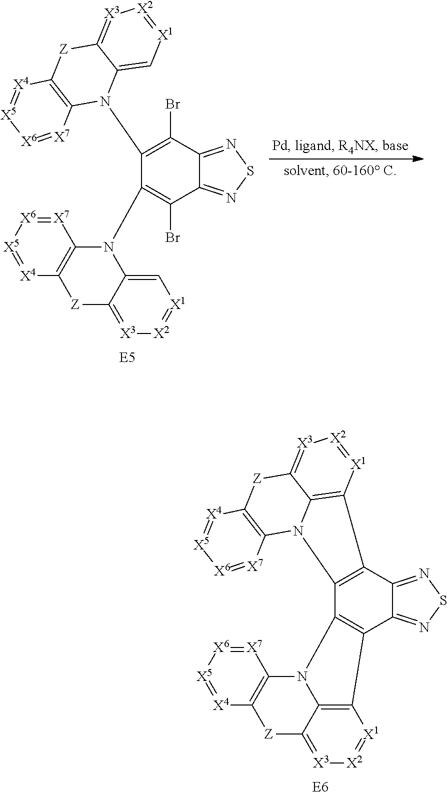

In the second step, C—H activation of E5 (1 eq) conducted in the presence of Pd^{II} or Pd⁰ catalyst (0.15 eq; for example Pd(OAc)₂, PdCl₂(PPh₃)₂, Pd(dppf)Cl₂, PdCl₂(PPh₃)₂, Pd(PPh₃)₄), ligand (0.4 eq; for example PPh₃, PCy₃, PCy₃—HBF₄, XPhos, S-Phos, R-Phos, xanphos, (tBu)₃P), quaternary ammonium salt (1 eq; for example TBACl, TBAB, TBAl, TBAOH, TMAB, THACl, TOACl, tetrabutylammonium tetrafluoroborate, benzyltrimethylammonium bromide), base (5 eq; for example K₂CO₃, K₃PO₄, NaH, NaOtBu, KOtBu, Cs₂CO₃, KOH, or NaOH), solvent (for example, DMAc, NMP, DMF, DME, MeCN, ToI, 1,4-dioxane, DMSO, EtOH, or THF) and at elevated temperature (60° C. or higher, and overnight) to achieve E6. Variations in the amount of compounds, phase catalyst, solvent, and temperature could be employed.

### General Procedure for Synthesis AAV3-3

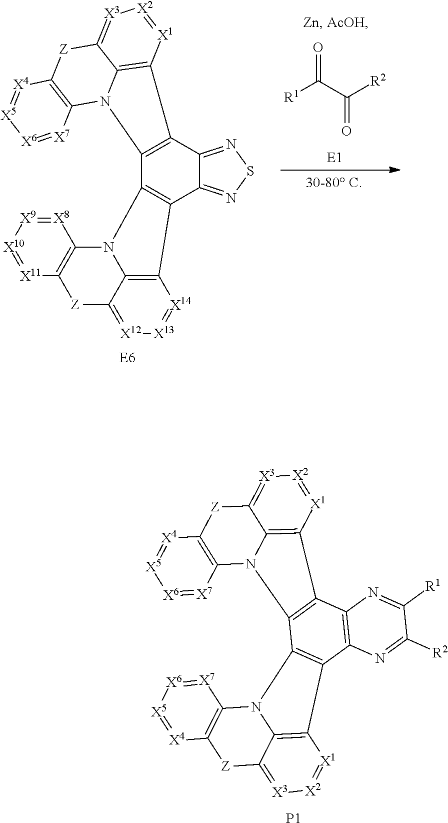

In the last step acetic acid (50 eq) added to a flask which contains E6 (1 eq) and Zn (10 eq) and the mixture stirred at 60° C. Then 1,2-dione compounds E1 were added to the obtained compound and the reaction was stirred at elevated temperature (30° C. or higher) to generate the corresponding organic molecules according to the invention, P1. Variations in the amount of compounds and temperature could be employed.

Cyclic Voltammetry

Cyclic voltammograms are measured from solutions having concentration of 10⁻³ mol/L of the organic molecules in dichloromethane or a suitable solvent and a suitable supporting electrolyte (e.g., 0.1 mol/L of tetrabutylammonium hexafluorophosphate). The measurements are conducted at room temperature under nitrogen atmosphere with a three-electrode assembly (Working and counter electrodes: Pt wire, reference electrode: Pt wire) and calibrated using FeCp₂/FeCp₂⁺ as internal standard. The HOMO data was corrected using ferrocene as internal standard against a saturated calomel electrode (SCE).

Density Functional Theory Calculation

Molecular structures are optimized employing the BP86 functional and the resolution of identity approach (RI). Excitation energies are calculated using the (BP86) optimized structures employing Time-Dependent DFT (TD-DFT) methods. Orbital and excited state energies are calculated with the B3LYP functional. Def2-SVP basis sets (and a m4-grid for numerical integration are used. The Turbomole program package is used for all calculations.

Photophysical Measurements
Sample pretreatment: Spin-coating
Apparatus: Spin150, SPS euro.
The sample concentration is 0.2 mg/ml, dissolved in Toluene/DCM.
Program: 7) 30 sec. at 2000 U/min. After coating, the films are dried at 70° C. for 1 min.

Fluorescence Spectroscopy and Phosphorescence Spectroscopy

For the analysis of Phosphorescence and Photoluminescence spectroscopy a fluorescence spectrometer “Fluoromax 4P” from Horiba is used.

Time-Resolved PL Spectroscopy in the μs-Range and ns-Range (FS5)

Time-resolved PL measurements were performed on an FS5 fluorescence spectrometer from Edinburgh Instruments. Compared to measurements on the HORIBA setup, better light gathering allows for an optimized signal to noise ratio, which favors the FS5 system especially for transient PL measurements of delayed fluorescence characteristics. The FS5 consists of a xenon lamp providing a broad spectrum. The continuous light source is a 150W xenon arc lamp, selected wavelengths are chosen by a Czerny-Turner monochromator, which is also used to set specific emission wavelengths. The sample emission is directed towards a sensitive R928P photomultiplier tube (PMT), allowing the detection of single photons with a peak quantum efficiency of up to 25% in the spectral range between 200 nm to 870 nm. The detector is a temperature stabilized PMT, providing dark counts below 300 cps (counts per second). Finally, to determine the transient decay lifetime of the delayed fluorescence, a tail fit using three exponential functions is applied. By weighting the specific lifetimes τᵢ with their corresponding amplitudes Aᵢ,

$${τ}_{DF} = {\sum }_{i = 1}^{3} \frac{{A}_{i} {τ}_{i}}{{A}_{i}}$$

the delayed fluorescence lifetime τ_{DF} is determined.

Photoluminescence Quantum Yield Measurements

For photoluminescence quantum yield (PLQY) measurements an Absolute PL Quantum Yield Measurement C₉₉₂₀-03G system (Hamamatsu Photonics) is used. Quantum yields and CIE coordinates are determined using the software U6039-05 version 3.6.0.

Emission maxima are given in nm, quantum yields Φ in % and CIE coordinates as x,y values.

PLQY is determined using the following protocol:

Quality assurance: Anthracene in ethanol (known concentration) is used as reference.

Excitation wavelength: the absorption maximum of the organic molecule is determined and the molecule is excited using this wavelength

Measurement

Quantum yields are measured, for sample, of solutions or films under nitrogen atmosphere. The yield is calculated using the equation:

$${Φ}_{PL} = \frac{{n}_{photon} , emitted}{{n}_{photon} , absorbed} = \frac{\int \frac{λ}{hc} [ {Int}_{emitted}^{sample} ( λ ) - {Int}_{absorbed}^{sample} ( λ ) ] d λ}{\int \frac{λ}{hc} [ {Int}_{emitted}^{reference} ( λ ) - {Int}_{absorbed}^{reference} ( λ ) ] d λ}$$

wherein nₚₕₒₜₒₙ denotes the photon count and Int. the intensity.

Production and Characterization of Optoelectronic Devices

Optoelectronic devices, such as OLED devices, including organic molecules according to the invention can be produced via vacuum-deposition methods. If a layer contains more than one compound, the weight-percentage of one or more compounds is given in %. The total weight-percentage values amount to 100%, thus if a value is not given, the fraction of this compound equals to the difference between the given values and 100%.

The not fully optimized OLEDs are characterized using standard methods and measuring electroluminescence spectra, the external quantum efficiency (in %) in dependency on the intensity, calculated using the light detected by the photodiode, and the current. The OLED device lifetime is extracted from the change of the luminance during operation at constant current density. The LT50 value corresponds to the time, where the measured luminance decreased to 50% of the initial luminance, analogously LT80 corresponds to the time point, at which the measured luminance decreased to 80% of the initial luminance, LT 95 to the time point, at which the measured luminance decreased to 95% of the initial luminance etc.

Accelerated lifetime measurements are performed (e.g., applying increased current densities). For example, LT80 values at 500 cd/m² are determined using the following equation:

$$LT 80 ( 500 \frac{{cd}^{2}}{{m}^{2}} ) = LT 80 ( {L}_{0} ) {( \frac{{L}_{0}}{500 \frac{{cd}^{2}}{{m}^{2}}} )}^{1.6}$$

wherein L₀ denotes the initial luminance at the applied current density.

The values correspond to the average of several pixels (typically two to eight), the standard deviation between these pixels is given.

HPLC-MS

HPLC-MS analysis is performed on an HPLC by Agilent (1260 series) with MS-detector (Thermo LTQ XL).

For example, a typical HPLC method is as follows: a reverse phase column 3.0 mm×100 mm, particle size 2.7 μm from Agilent (Poroshell 120EC-C18, 3.0×100 mm, 2.7 μm HPLC column) is used in the HPLC. The HPLC-MS measurements are performed at room temperature (rt) following gradients

| Flow rate [ml/min] | Time [min] | A[%] | B[%] | C[%] |
|---|---|---|---|---|
| 1.5 | 30 | 40 | 40 | 30 |
| 1.5 | 45 | 10 | 10 | 80 |
| 1.5 | 50 | 40 | 10 | 80 |
| 1.5 | 51 | 30 | 40 | 30 |
| 1.5 | 55 | 30 | 10 | 30 |

using the following solvent mixtures containing 0.1% formic acid:

| | | |
|---|---|---|
| Solvent A: | H2O (10%) | MeCN (90%) |
| Solvent B: | H2O (90%) | MeCN (10%) |
| Solvent C: | THF (50%) | MeCN (50%) |

An injection volume of 2 μL from a solution with a concentration of 0.5 mg/mL of the analyte is taken for the measurements.

Ionization of the probe is performed using an atmospheric pressure chemical ionization (APCI) source either in positive (APCI+) or negative (APCI−) ionization mode or an atmospheric pressure photoionization (APPI) source.

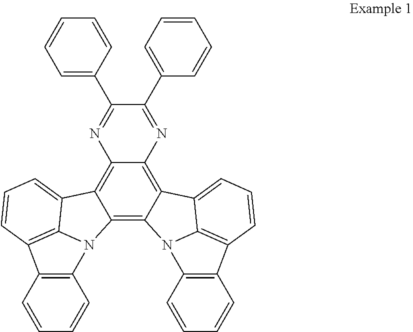 [SMILES: c1ccc(-c2nc3c(nc2-c2ccccc2)c2c4cccc5c6ccccc6n(c54)c2c2c3c3cccc4c5ccccc5n2c43)cc1]

Example 1 was synthesized according to
AAV1 (69% yield), wherein benzil [134-81-6] was used as E1, reaction temperature set to 40° C. and reaction time of 16 hours;
AAV2 (82% yield), wherein carbazole [201-696-0] was used as E3, DMF as solvent, potassium carbonate [584-08-7] as base, reaction temperature set to 80° C. and reaction time of 24 h; and
AAV3 (80% yield), wherein Pd(OAC)₂ [3375-31-3], PPh₃ [603-35-0], tetrabutylammonium bromide [1643-19-2 were used as catalyst system, potassium carbonate [584-08-7] as base, N,N-dimethyl acetamide as a solvent, reaction temperature set to 140° C. and reaction time of 24 h.

The drawing depicts the emission spectrum of example 1 (0,001 mg/ml in toluene). The emission maximum (λₘₐₓ) is at 461 nm. The photoluminescence quantum yield (PLQY) is 68%, the full width at half maximum (FWHM) is 0.22 eV. The resulting CIEₓ coordinate is 0.13 and the CIE_{y} coordinate is 0.15.

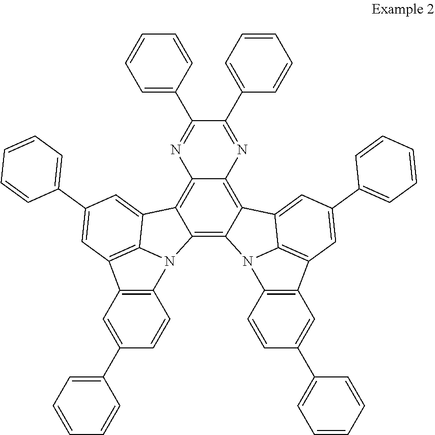 [SMILES: c1ccc(-c2ccc3c(c2)c2cc(-c4ccccc4)cc4c5c6nc(-c7ccccc7)c(-c7ccccc7)nc6c6c7cc(-c8ccccc8)cc8c9cc(-c%10ccccc%10)ccc9n(c87)c6c5n3c24)cc1]

Example 2 was synthesized according to
AAV1 (69% yield), wherein benzil [134-81-6] was used as E1, reaction temperature set to 40° C. and reaction time of 16 hours;
AAV2 (96% yield), wherein 3,6-diphenyl-9H-carbazole [56525-79-2] was used as E3, DMF as solvent, potassium carbonate [584-08-7] as base, reaction temperature set to 80° C. and reaction time of 24 h; and
AAV3 (73% yield), wherein Pd(OAC)₂ [3375-31-3], PPh₃ [603-35-0], tetrabutylammonium bromide [1643-19-2 were used as catalyst system, potassium carbonate [584-08-7] as base, N,N-dimethyl acetamide as a solvent, reaction temperature set to 140° C. and reaction time of 36 h.

The emission spectrum of example 2 (0.001 mg/ml in DCM) has an emission maximum (λₘₐₓ) at 477 nm. The photoluminescence quantum yield (PLQY) is 70%, the full width at half maximum (FWHM) is 0.21 eV. The resulting CIEₓ coordinate is 0.129 and the CIE_{y} coordinate is 0.344.

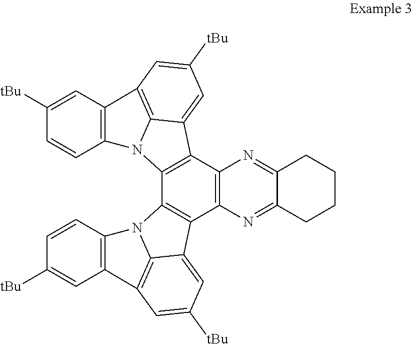 [SMILES: CC(C)(C)c1ccc2c(c1)c1cc(C(C)(C)C)cc3c4c5nc6c(nc5c5c7cc(C(C)(C)C)cc8c9cc(C(C)(C)C)ccc9n(c87)c5c4n2c13)CCCC6]

Example 3 was synthesized according to
AAV3-1 (37% yield), wherein 3,6-Di-tert-butylcarbazole [37500-95-1] was used as E3, DMF as solvent, potassium carbonate [584-08-7] as base, reaction temperature set to 100° C. and reaction time of 100 h; and
AAV3-2 (54% yield), wherein Pd(OAC)₂ [3375-31-3], PPh₃ [603-35-0], tetrabutylammonium bromide [1643-19-2] were used as catalyst system, potassium carbonate [584-08-7] as base, N,N-dimethyl acetamide as a solvent, reaction temperature set to 140° C. and reaction time of 3 h; and
AAV3-3 (22% yield), wherein cyclohexane-1,2-dione [765-87-7] was used as E1, reaction temperature set to 40° C. and reaction time of 11 hours.

The emission spectrum of example 3 (0.001 mg/ml in toluene) has an emission maximum (λₘₐₓ) at 450 nm. The photoluminescence quantum yield (PLQY) is 57%, the full width at half maximum (FWHM) is 0.15 eV. The resulting CIEₓ coordinate is 0.139 and the CIE_{y} coordinate is 0.104.

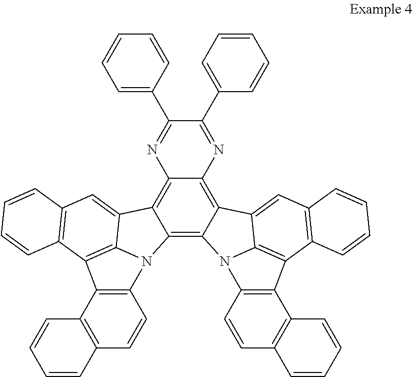 [SMILES: c1ccc(-c2nc3c(nc2-c2ccccc2)c2c4cc5ccccc5c5c6c7ccccc7ccc6n(c45)c2c2c3c3cc4ccccc4c4c5c6ccccc6ccc5n2c34)cc1]

Example 4 was synthesized according to
AAV1 (69% yield), wherein benzil [134-81-6] was used as E1, reaction temperature set to 40° C. and reaction time of 16 hours;
AAV2 (58% yield), wherein 7H-dibenzo[c,g]carbazole [194-59-2] was used as E3, DMF as solvent, potassium carbonate [584-08-7] as base, reaction temperature set to 100° C. and reaction time of 24 h; and
AAV3 (21% yield), wherein Pd(OAC)₂ [3375-31-3], PPh₃ [603-35-0], tetrabutylammonium bromide [1643-19-2 were used as catalyst system, potassium carbonate [584-08-7] as base, N,N-dimethyl acetamide as a solvent, reaction temperature set to 140° C. and reaction time of 72 h.

The emission spectrum of example 4 (0.001 mg/ml in toluene) has an emission maximum (λₘₐₓ) at 501 nm. The full width at half maximum (FWHM) is 0.13 eV. The resulting CIEₓ coordinate is 0.21 and the CIE_{y} coordinate is 0.59.

### ADDITIONAL EXAMPLES OF ORGANIC MOLECULES OF THE INVENTION

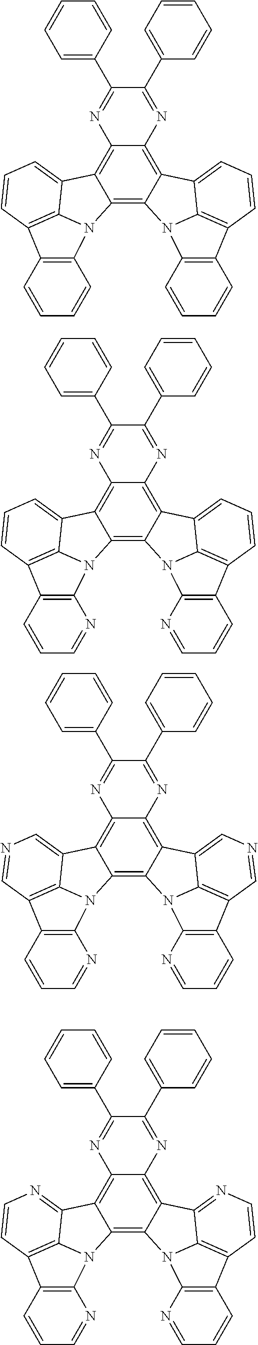

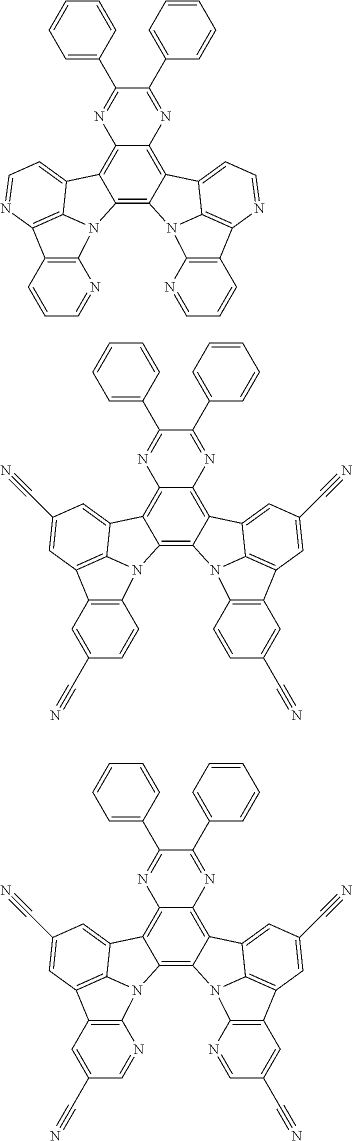

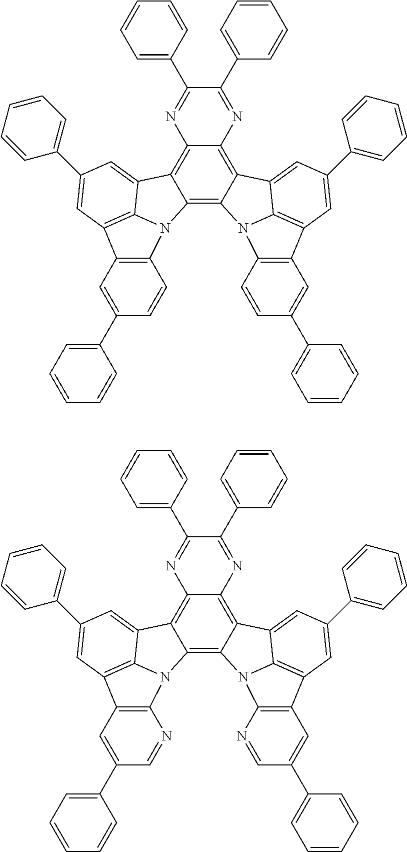 [SMILES: c1ccc(-c2ccc3c(c2)c2cc(-c4ccccc4)cc4c5c6nc(-c7ccccc7)c(-c7ccccc7)nc6c6c7cc(-c8ccccc8)cc8c9cc(-c%10ccccc%10)ccc9n(c87)c6c5n3c24)cc1 ; c1ccc(-c2cnc3c(c2)c2cc(-c4ccccc4)cc4c5c6nc(-c7ccccc7)c(-c7ccccc7)nc6c6c7cc(-c8ccccc8)cc8c9cc(-c%10ccccc%10)cnc9n(c87)c6c5n3c24)cc1]

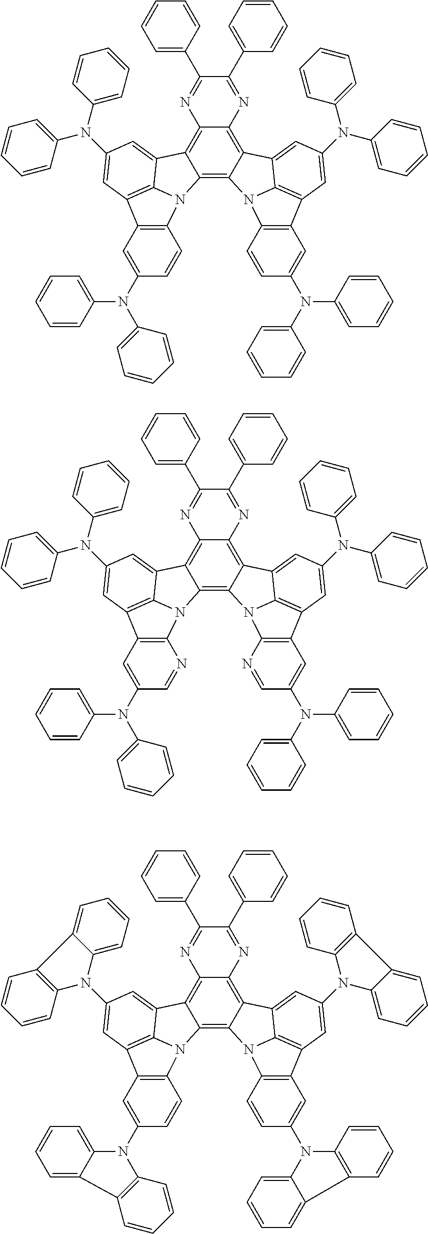

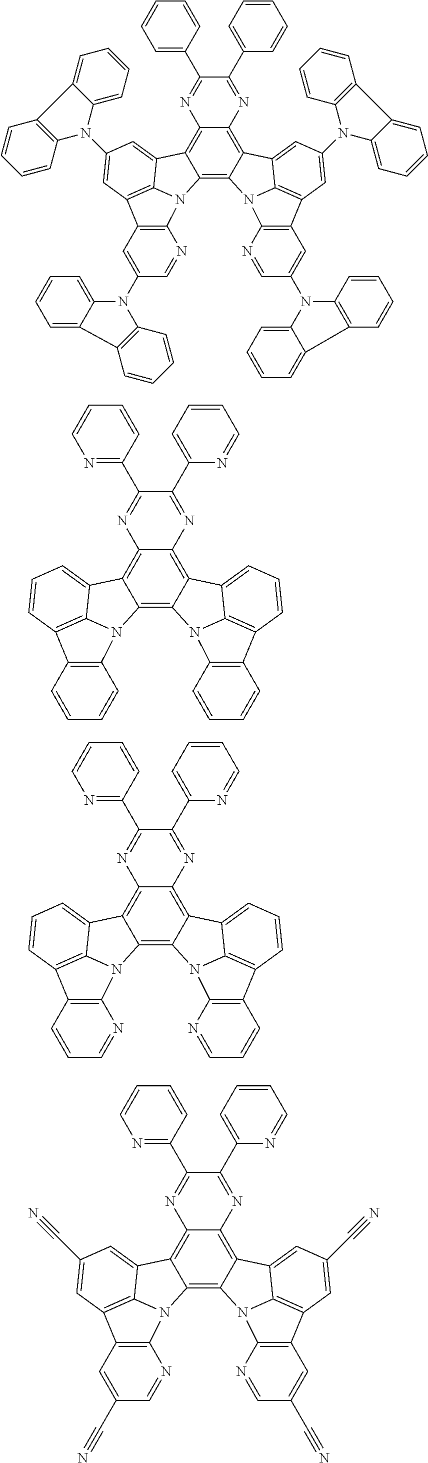

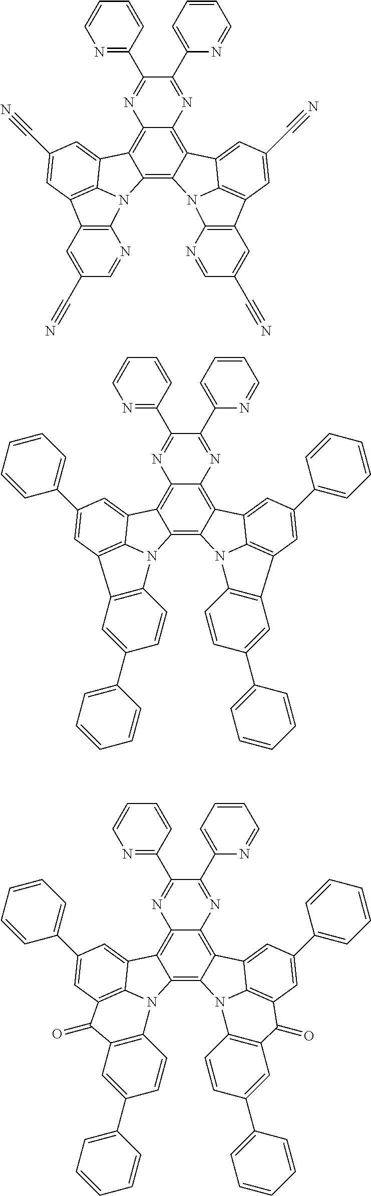

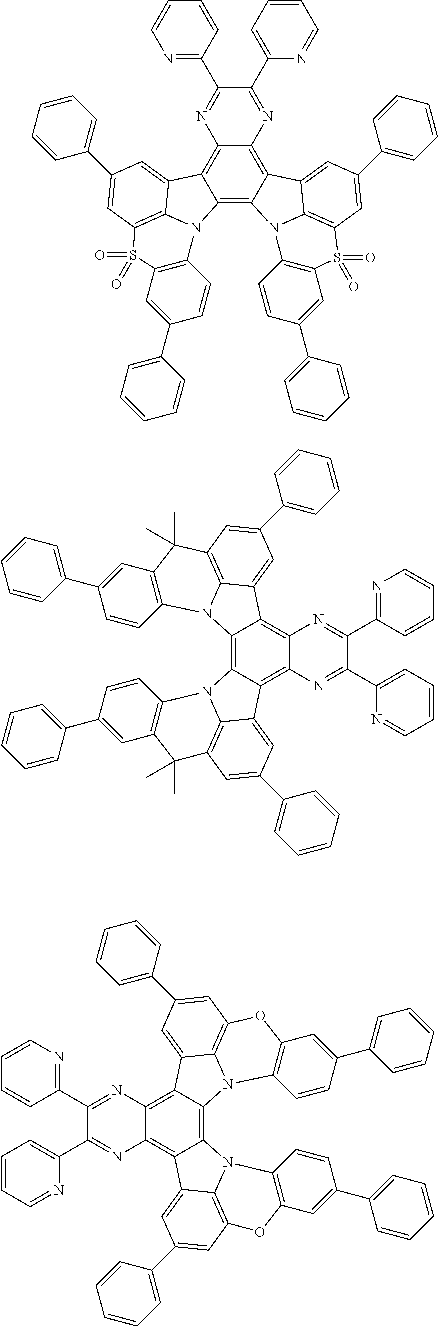

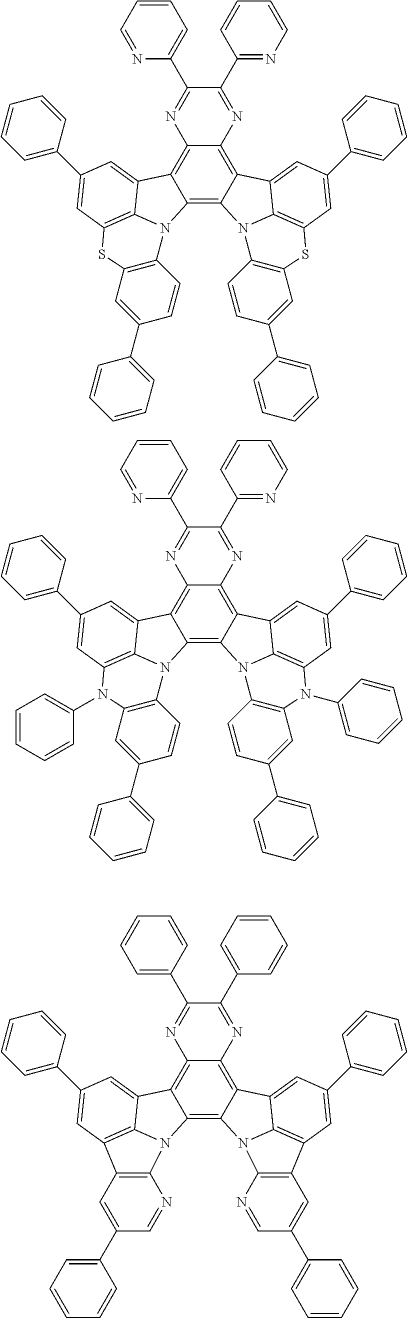

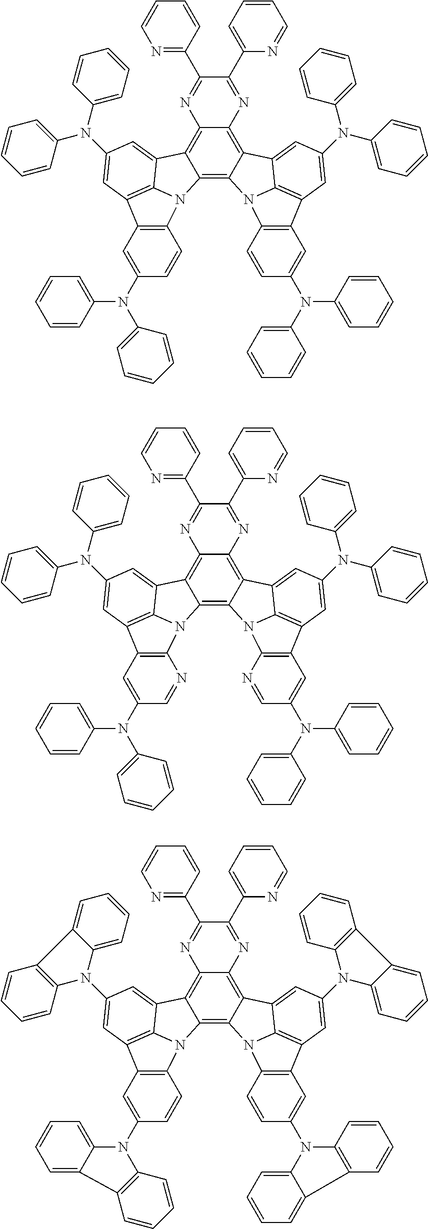

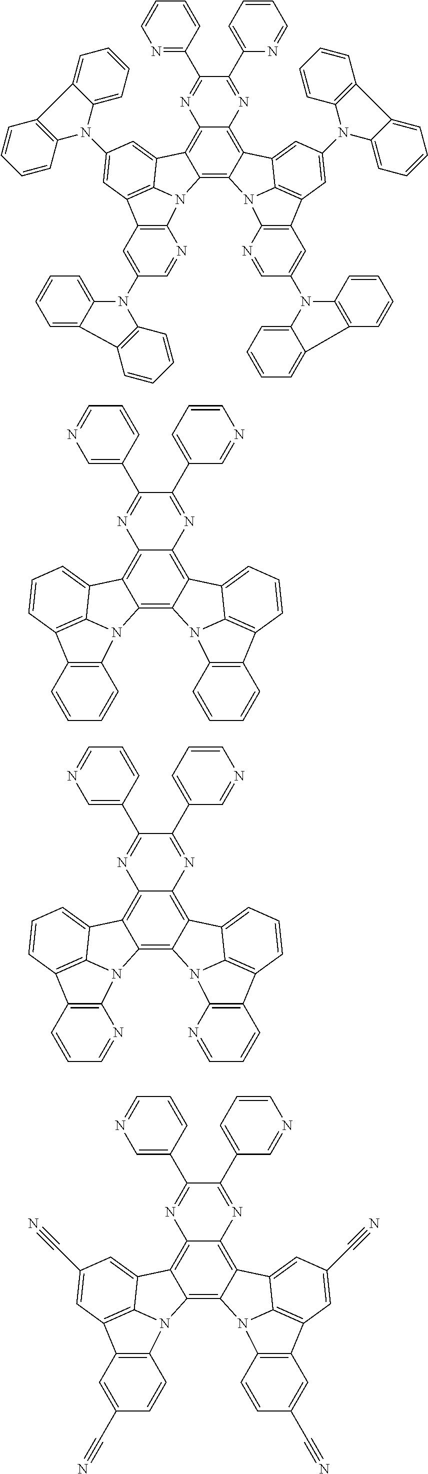

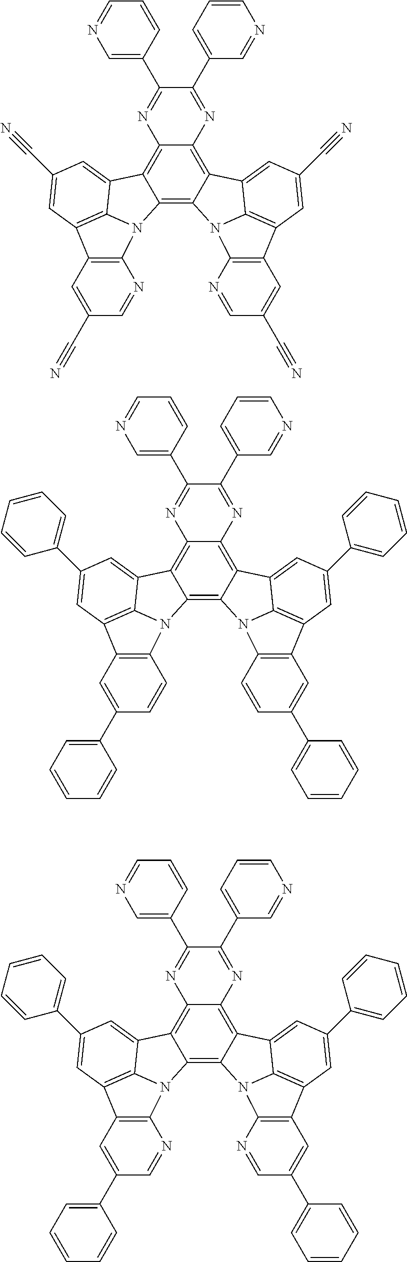

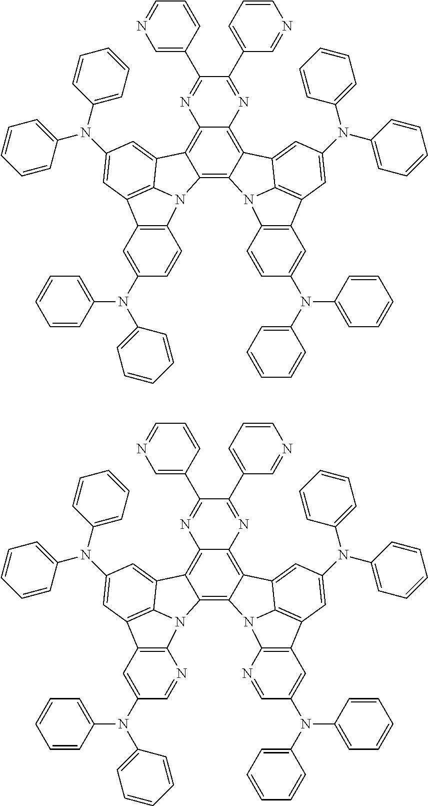

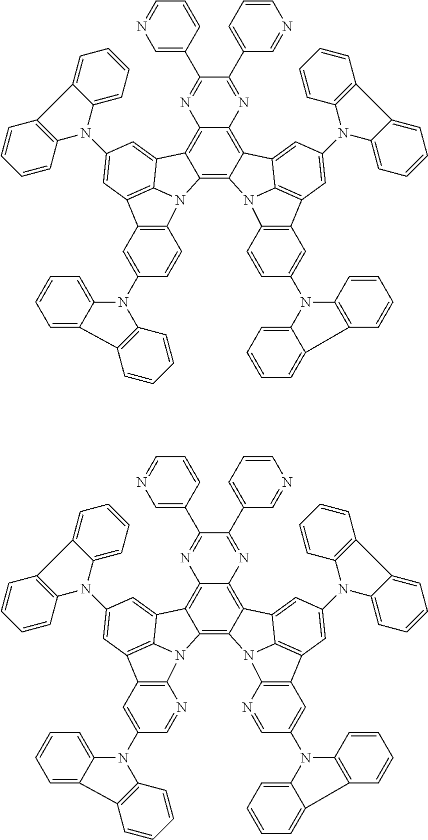 [SMILES: c1cncc(-c2nc3c(nc2-c2cccnc2)c2c4cc(-n5c6ccccc6c6ccccc65)cc5c6cc(-n7c8ccccc8c8ccccc87)ccc6n(c54)c2c2c3c3cc(-n4c5ccccc5c5ccccc54)cc4c5cc(-n6c7ccccc7c7ccccc76)ccc5n2c43)c1 ; c1cncc(-c2nc3c(nc2-c2cccnc2)c2c4cc(-n5c6ccccc6c6ccccc65)cc5c6cc(-n7c8ccccc8c8ccccc87)cnc6n(c54)c2c2c3c3cc(-n4c5ccccc5c5ccccc54)cc4c5cc(-n6c7ccccc7c7ccccc76)cnc5n2c43)c1]

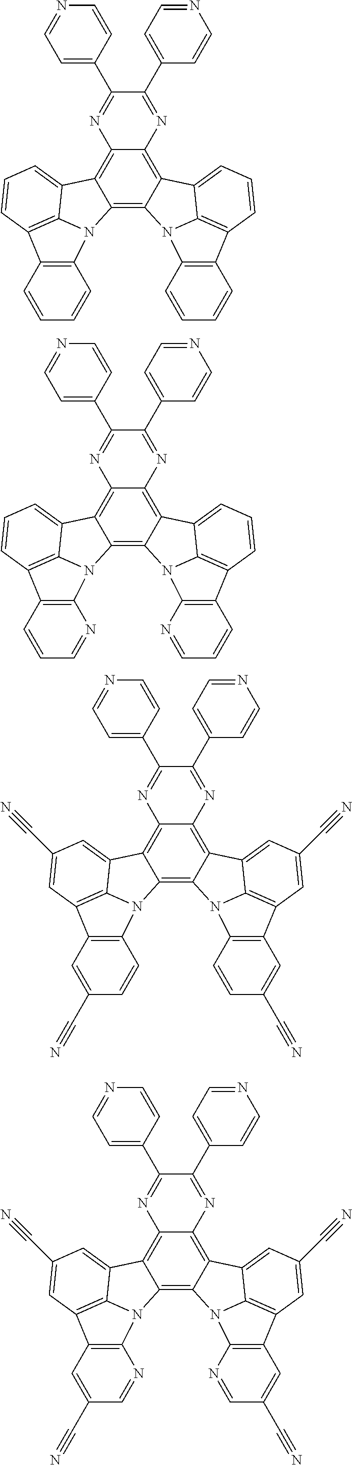

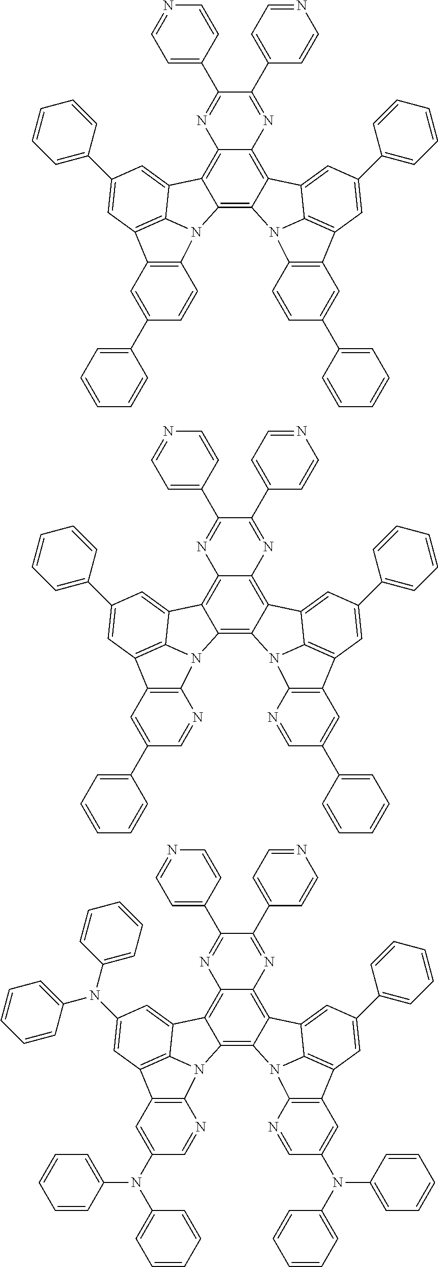

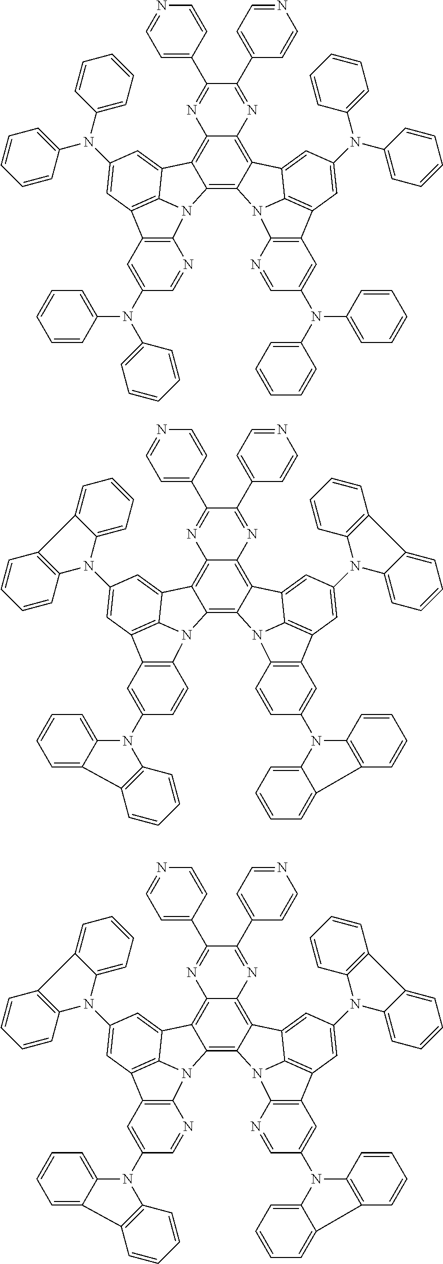

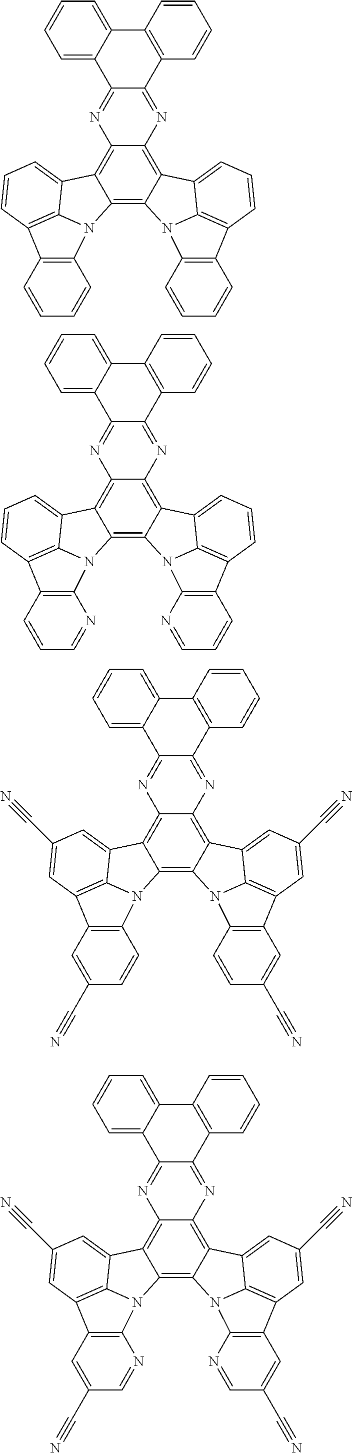

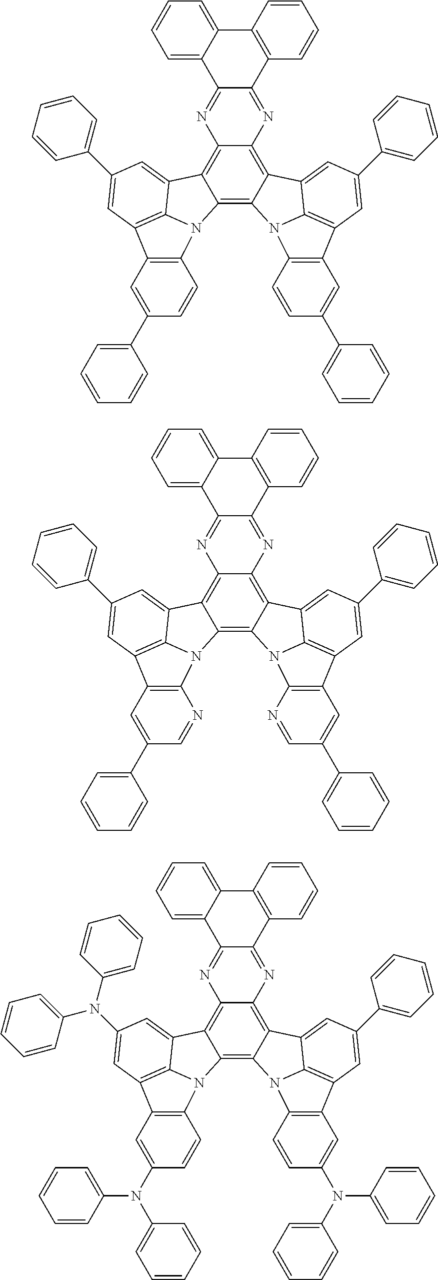

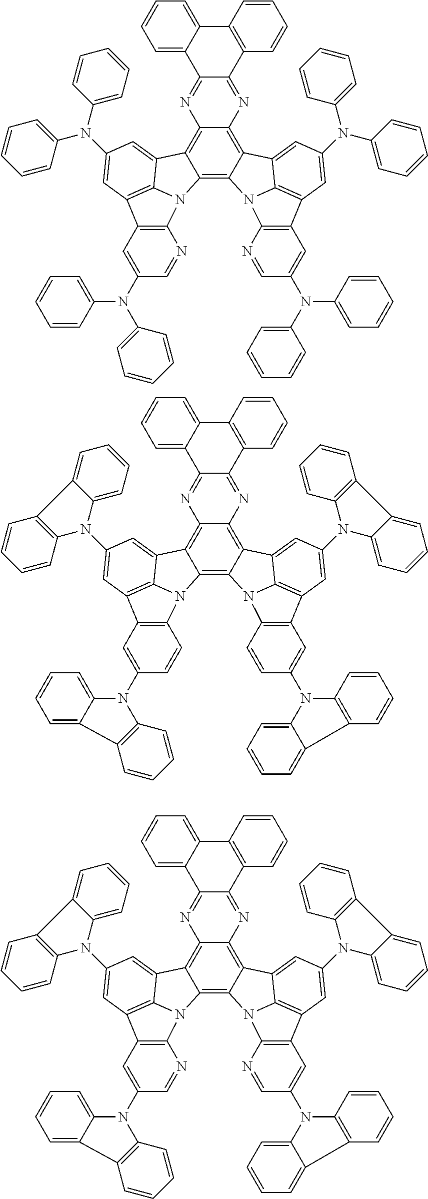

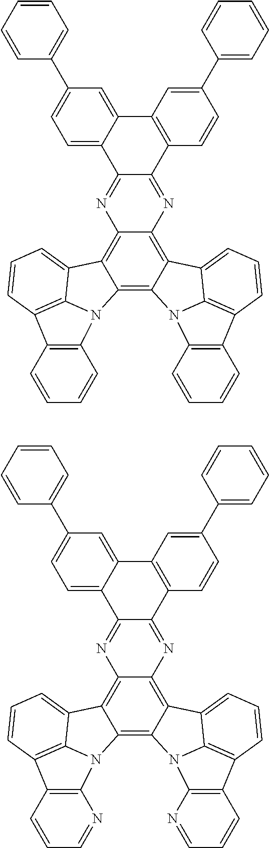

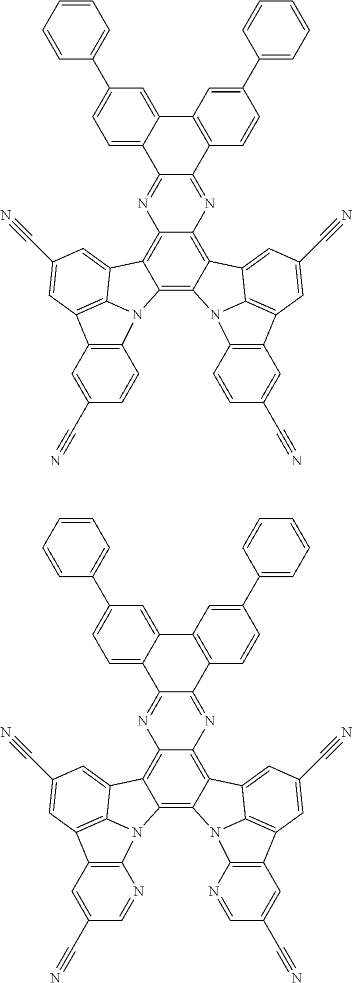 [SMILES: N#Cc1ccc2c(c1)c1cc(C#N)cc3c4c5nc6c7ccc(-c8ccccc8)cc7c7cc(-c8ccccc8)ccc7c6nc5c5c6cc(C#N)cc7c8cc(C#N)ccc8n(c76)c5c4n2c13 ; N#Cc1cnc2c(c1)c1cc(C#N)cc3c4c5nc6c7ccc(-c8ccccc8)cc7c7cc(-c8ccccc8)ccc7c6nc5c5c6cc(C#N)cc7c8cc(C#N)cnc8n(c76)c5c4n2c13]

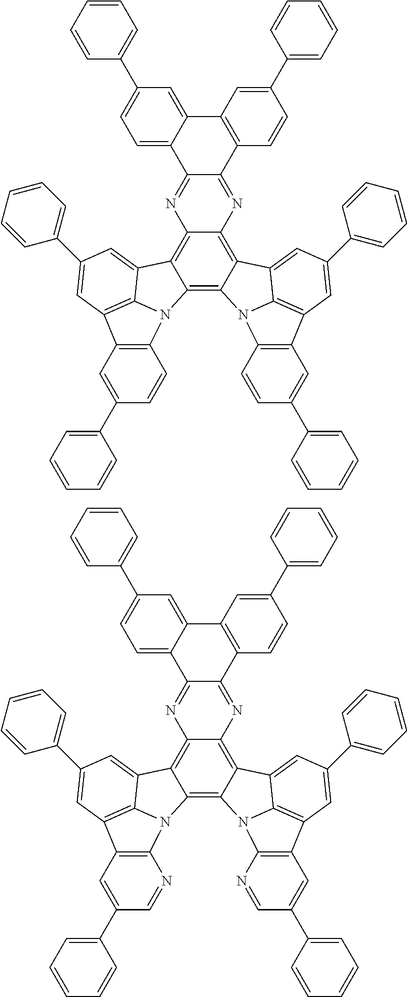

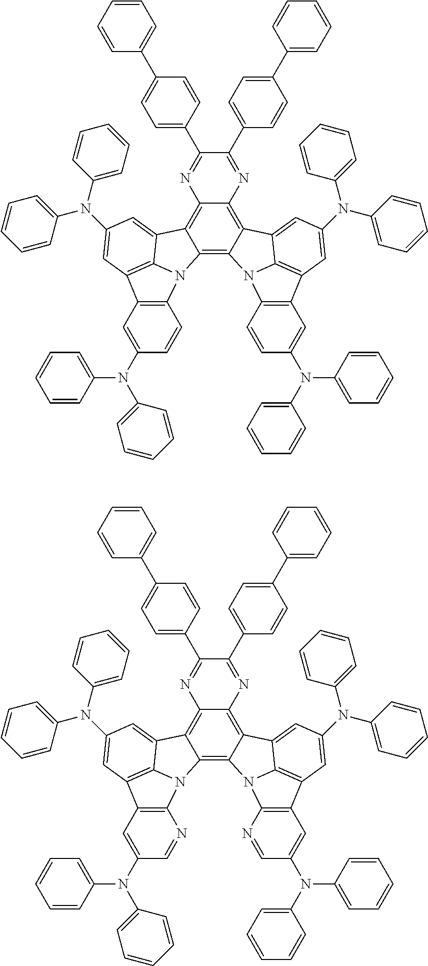 [SMILES: c1ccc(-c2ccc(-c3nc4c(nc3-c3ccc(-c5ccccc5)cc3)c3c5cc(N(c6ccccc6)c6ccccc6)cc6c7cc(N(c8ccccc8)c8ccccc8)ccc7n(c65)c3c3c4c4cc(N(c5ccccc5)c5ccccc5)cc5c6cc(N(c7ccccc7)c7ccccc7)ccc6n3c54)cc2)cc1 ; c1ccc(-c2ccc(-c3nc4c(nc3-c3ccc(-c5ccccc5)cc3)c3c5cc(N(c6ccccc6)c6ccccc6)cc6c7cc(N(c8ccccc8)c8ccccc8)cnc7n(c65)c3c3c4c4cc(N(c5ccccc5)c5ccccc5)cc5c6cc(N(c7ccccc7)c7ccccc7)cnc6n3c54)cc2)cc1]

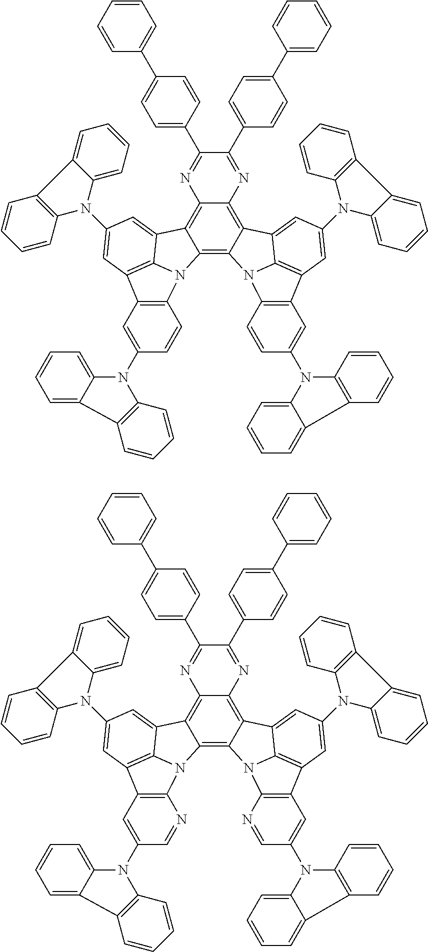

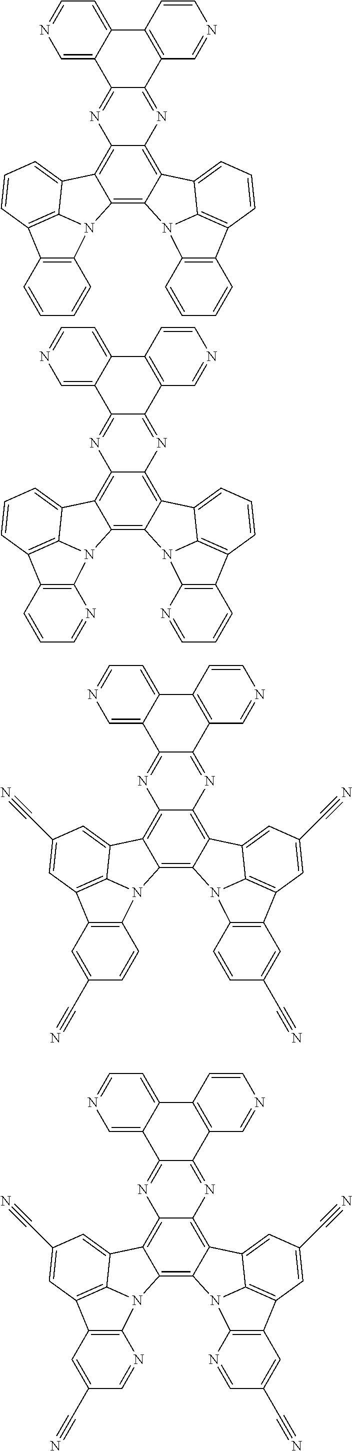

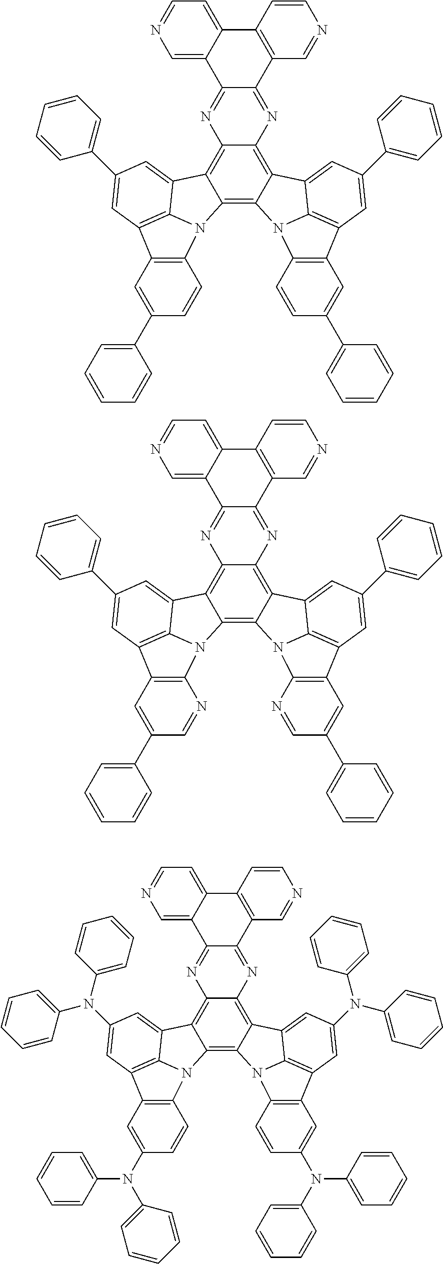

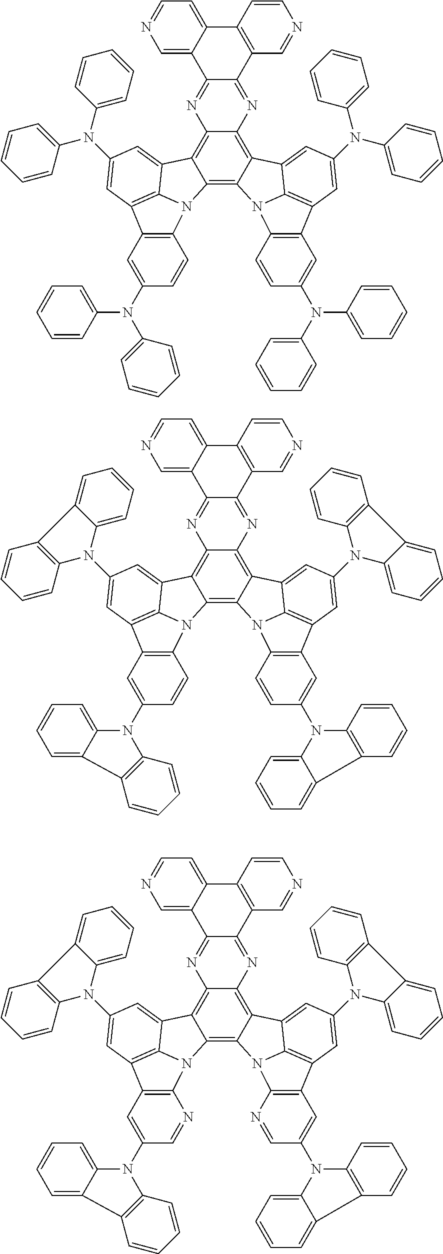

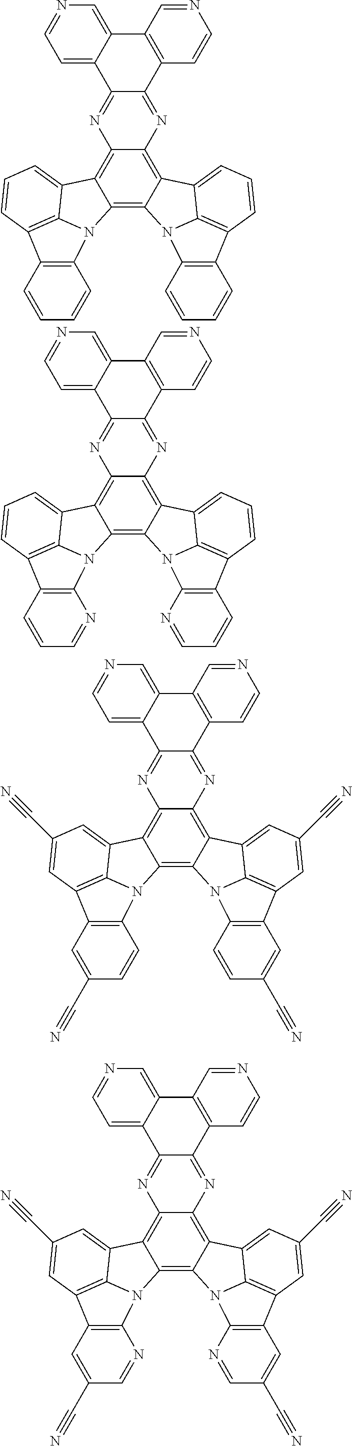

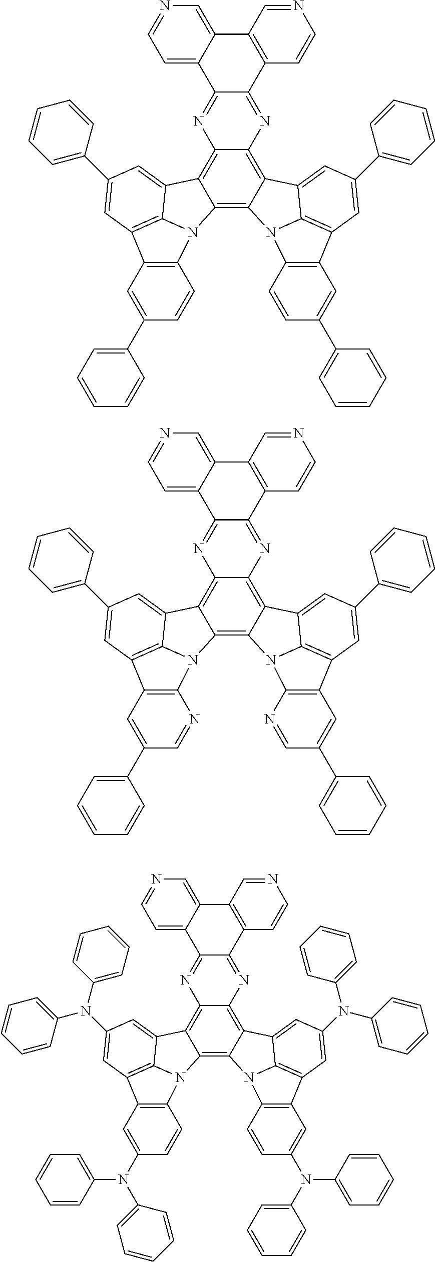

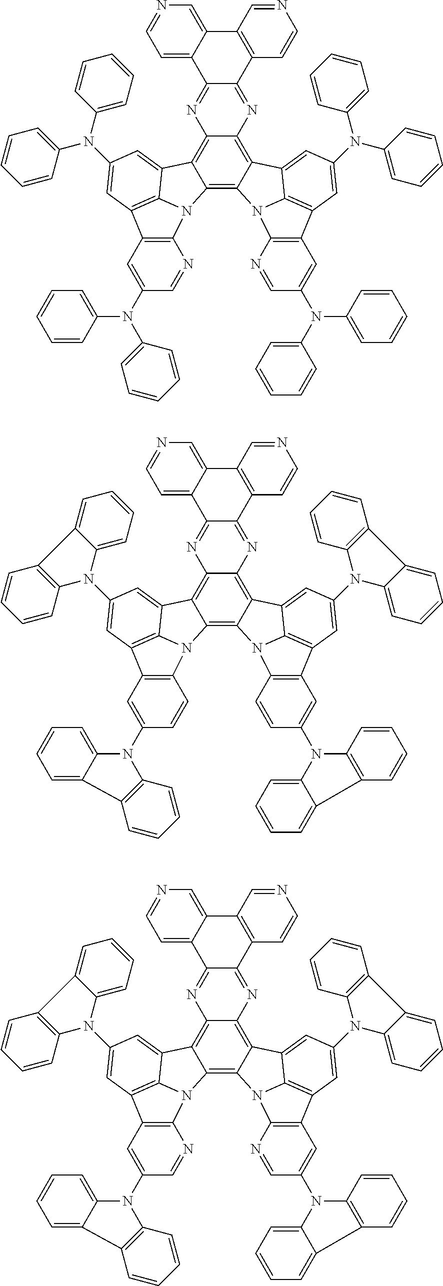

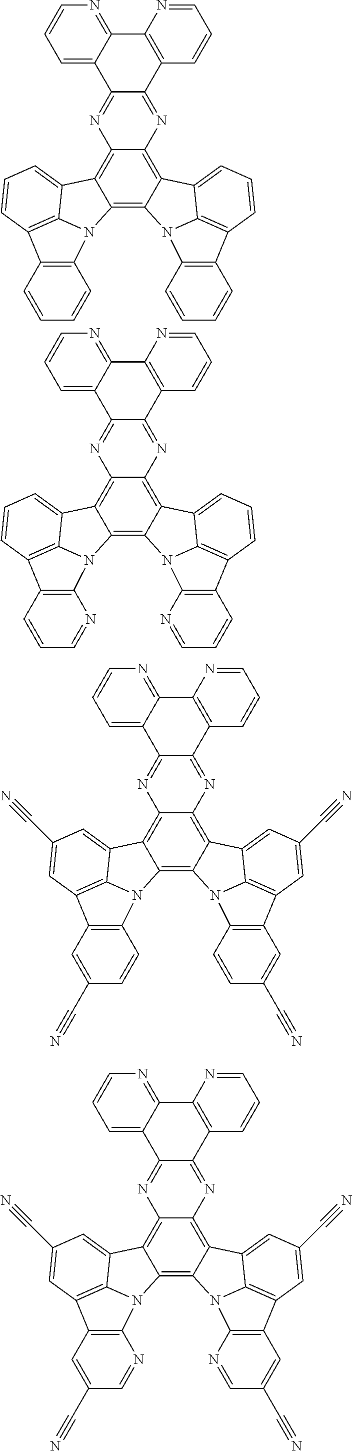

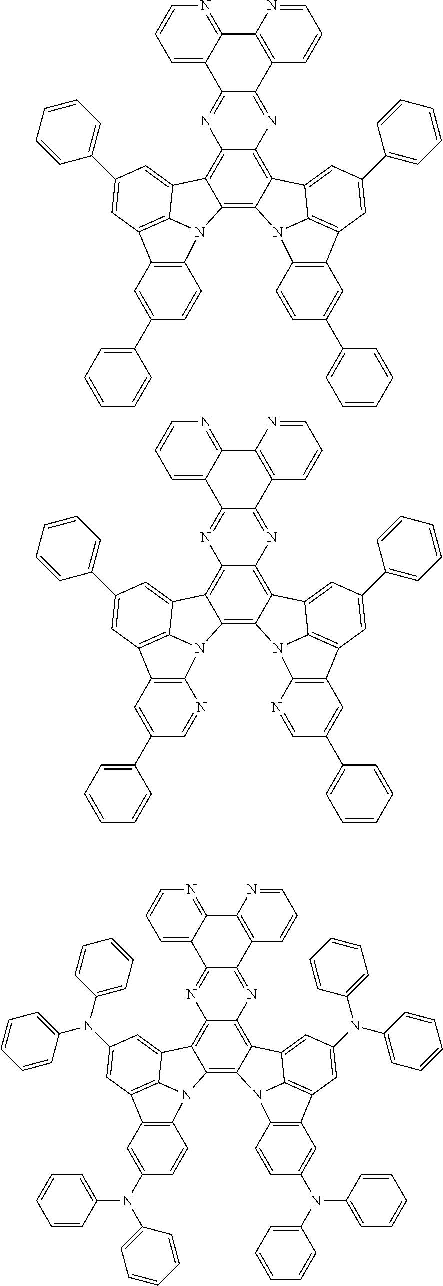

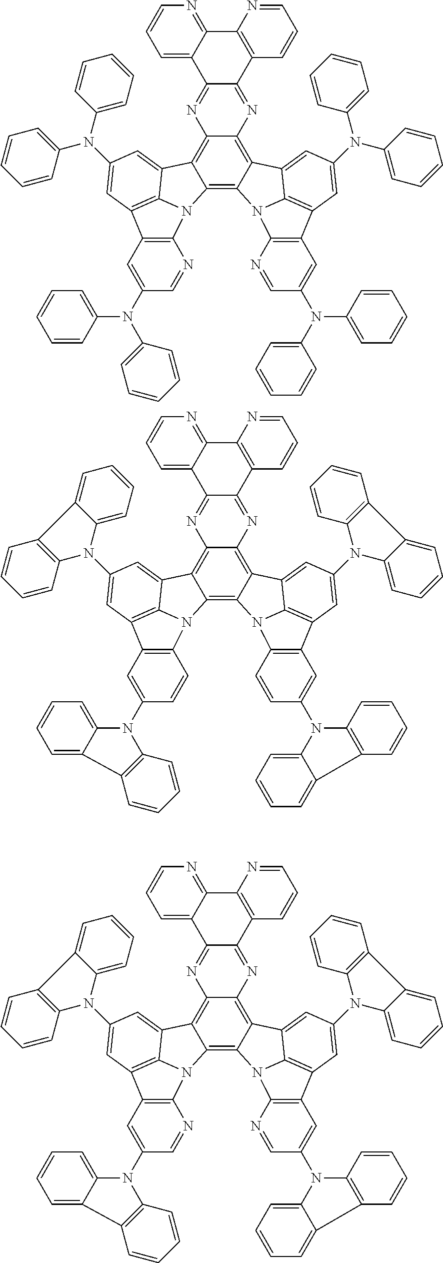

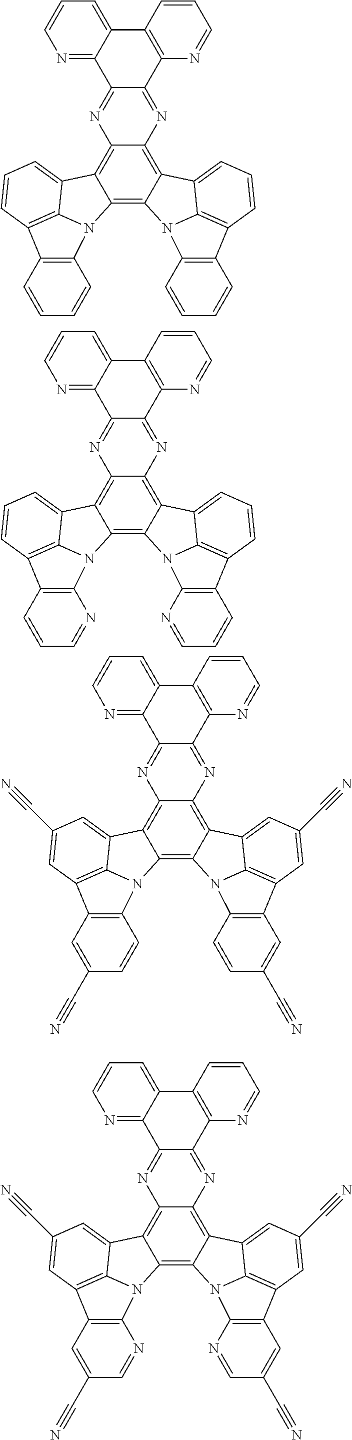

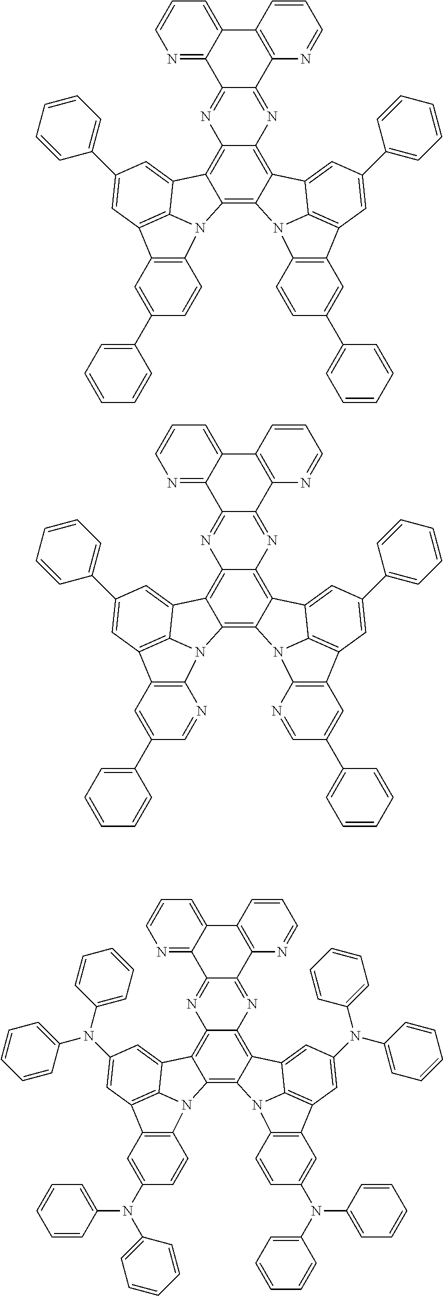

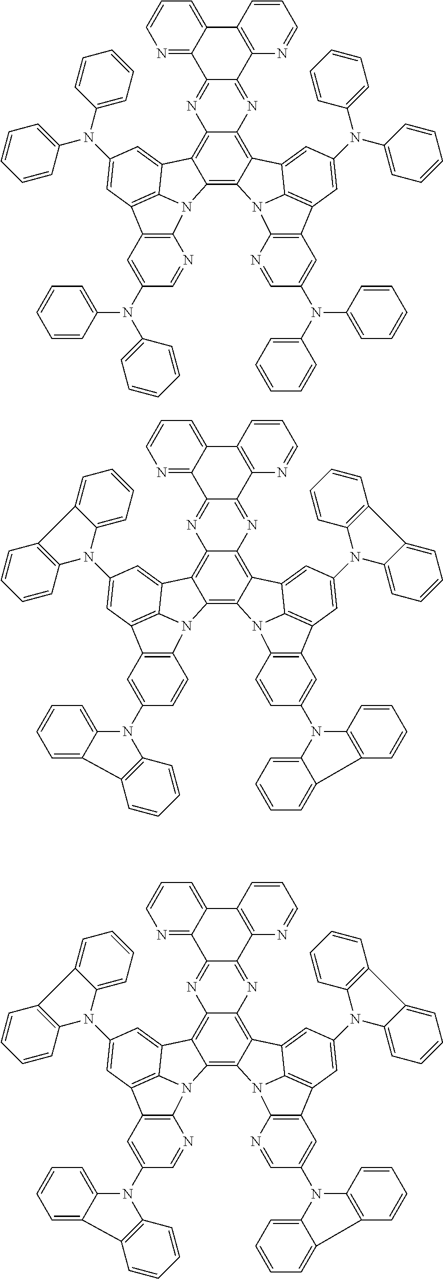

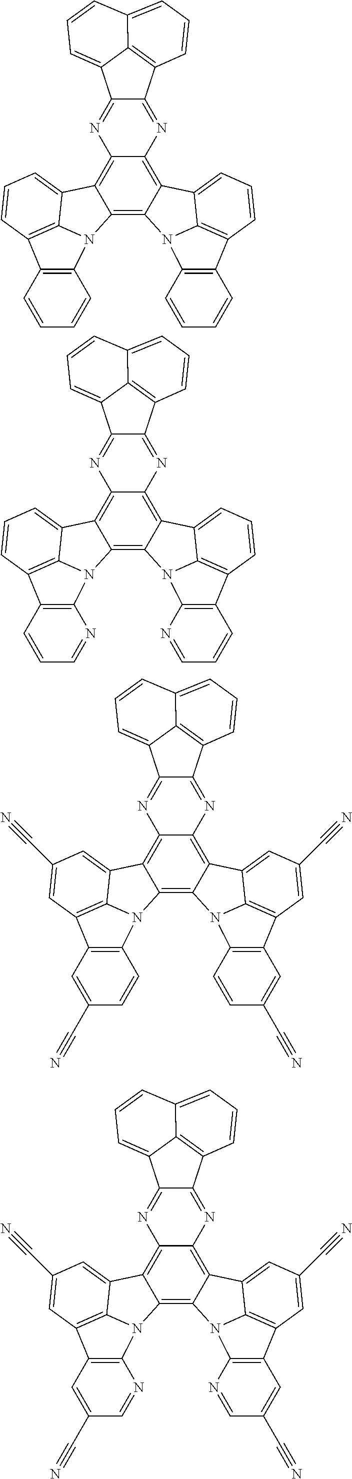

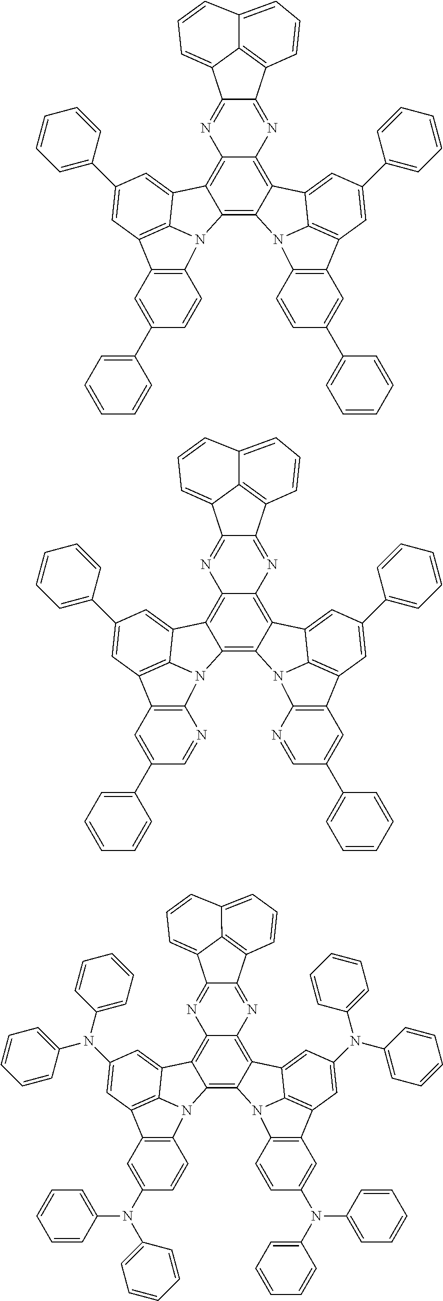 [SMILES: c1ccc(-c2ccc3c(c2)c2cc(-c4ccccc4)cc4c5c6nc7c8cccc9cccc(c7nc6c6c7cc(-c%10ccccc%10)cc%10c%11cc(-c%12ccccc%12)ccc%11n(c%107)c6c5n3c24)c98)cc1 ; c1ccc(-c2cnc3c(c2)c2cc(-c4ccccc4)cc4c5c6nc7c8cccc9cccc(c7nc6c6c7cc(-c%10ccccc%10)cc%10c%11cc(-c%12ccccc%12)cnc%11n(c%107)c6c5n3c24)c98)cc1 ; c1ccc(N(c2ccccc2)c2ccc3c(c2)c2cc(N(c4ccccc4)c4ccccc4)cc4c5c6nc7c8cccc9cccc(c7nc6c6c7cc(N(c%10ccccc%10)c%10ccccc%10)cc%10c%11cc(N(c%12ccccc%12)c%12ccccc%12)ccc%11n(c%107)c6c5n3c24)c98)cc1]

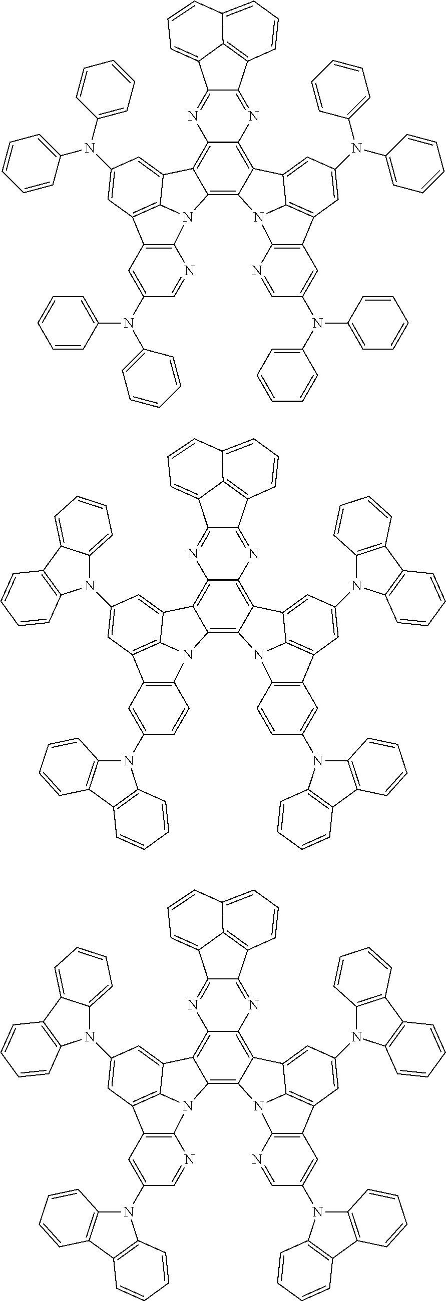

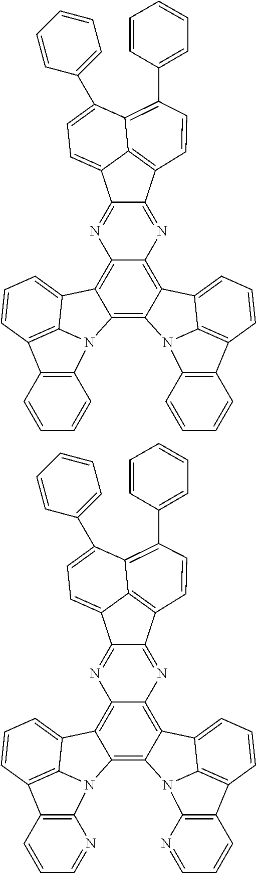

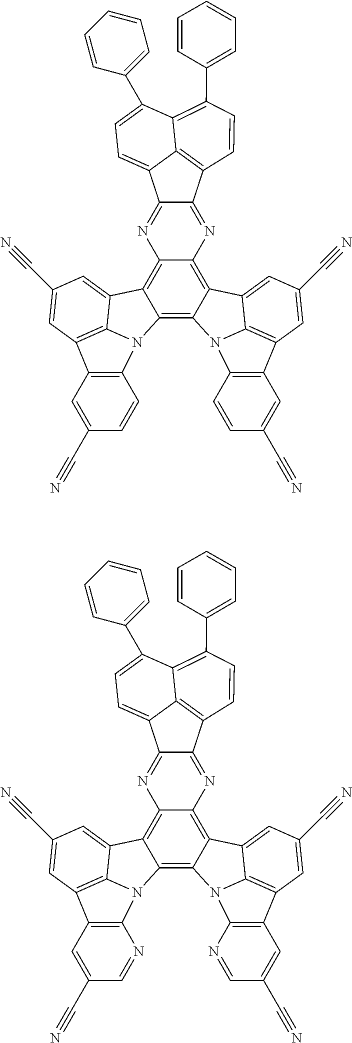 [SMILES: N#Cc1ccc2c(c1)c1cc(C#N)cc3c4c5nc6c7ccc(-c8ccccc8)c8c(-c9ccccc9)ccc(c6nc5c5c6cc(C#N)cc9c%10cc(C#N)ccc%10n(c96)c5c4n2c13)c87 ; N#Cc1cnc2c(c1)c1cc(C#N)cc3c4c5nc6c7ccc(-c8ccccc8)c8c(-c9ccccc9)ccc(c6nc5c5c6cc(C#N)cc9c%10cc(C#N)cnc%10n(c96)c5c4n2c13)c87]

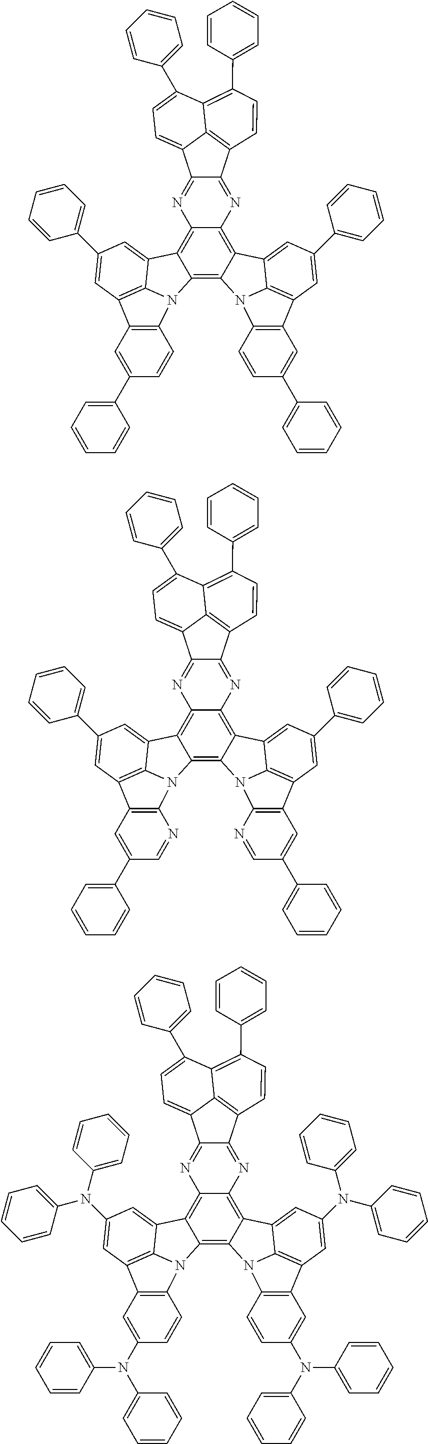

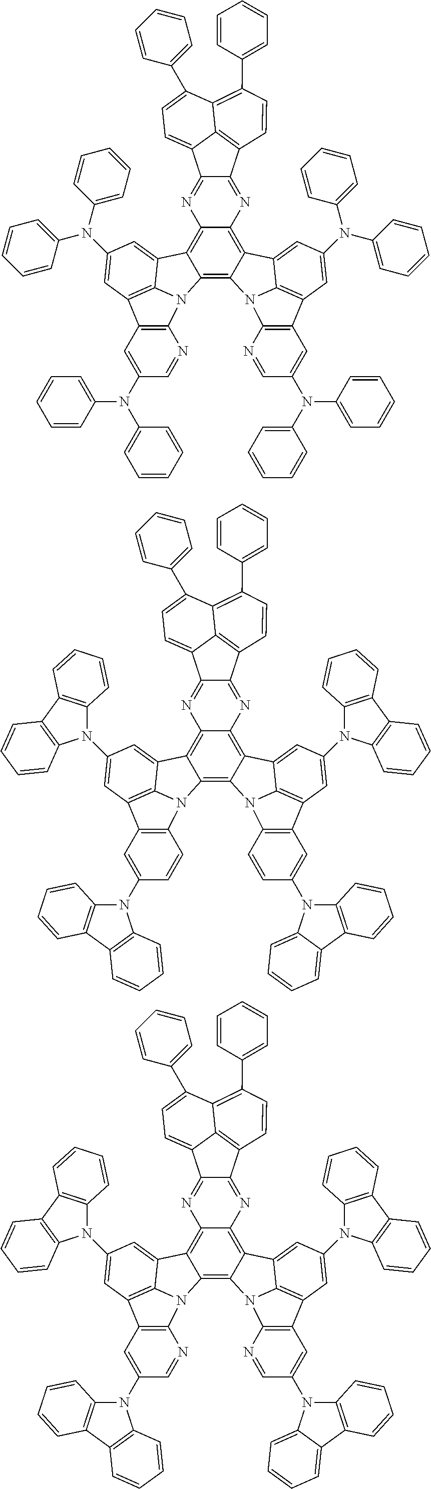

### BRIEF DESCRIPTION OF THE DRAWINGS

The drawing illustrates an emission spectrum of example 1 (0.001 mg/ml) in toluene.

1-15. (canceled)

16. An organic molecule, comprising a structure of Formula I: 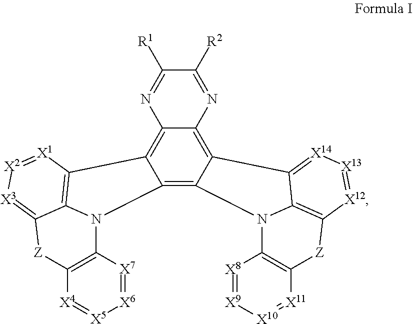
wherein
X¹, X², X³, X⁴, X⁵, X⁶, X⁷, X⁸, X⁹, X¹⁰, X¹¹, X¹², X¹³, and X¹⁴ is independently from each other selected from the group consisting of N and CR^{a};
Z is at each occurrence independently from one another selected from the group consisting of a direct bond, CR³R⁴, C═CR³R⁴, C═O, C═NR³, NR³, O, SiR³R⁴, S, S(O) and S(O)₂;
R¹ and R² is at each occurrence independently from one another selected from the group consisting of hydrogen; deuterium; N(R⁵)₂; OR⁵; Si(R⁵)₃; B(OR⁵)₂; OSO₂R⁵;
CF₃; CN; F; Br; I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵; and
a mono- or polycyclic, aliphatic, aromatic and/or benzo-fused ring system formed by ring-closure with one or more other substituent selected from the group consisting of R¹, R² and R⁵;
R^{a}, R³ and R⁴ is at each occurrence independently from one another selected from the group consisting of hydrogen;
deuterium;
N(R⁵)₂;
OR⁵;
Si(R⁵)₃,
B(OR⁵)₂;
OSO₂R⁵;
CF₃;
CN;
F;
Br;
I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵;
R⁵ is at each occurrence independently from one another selected from the group consisting of hydrogen; deuterium; N(R⁶)₂; OR⁶; Si(R⁶)₃; B(OR⁶)₂; OSO₂R⁶; CF₃; CN; F; Br; I;
C₁-C₄₀-alkyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-alkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-thioalkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkenyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkynyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₆-C₆₀-aryl,
which is optionally substituted with one or more substituents R⁶; and
C₃-C₅₇-heteroaryl,
which is optionally substituted with one or more substituents R⁶;
R⁶ is at each occurrence independently from one another selected from the group consisting of hydrogen; deuterium; OPh; CF₃; CN; F;
C₁-C₅-alkyl,
wherein one or more hydrogen atoms are optionally, independently from each other substituted by deuterium, CN, CF₃, or F;
C₁-C₅-alkoxy,
wherein one or more hydrogen atoms are optionally, independently from each other substituted by deuterium, CN, CF₃, or F;
C₁-C₅-thioalkoxy,
wherein one or more hydrogen atoms are optionally, independently from each other substituted by deuterium, CN, CF₃, or F;
C₂-C₅-alkenyl,
wherein one or more hydrogen atoms are optionally, independently from each other substituted by deuterium, CN, CF₃, or F;
C₂-C₅-alkynyl,
wherein one or more hydrogen atoms are optionally, independently from each other substituted by deuterium, CN, CF₃, or F;
C₆-C₁₈-aryl,
which is optionally substituted with one or more C₁-C₅-alkyl substituents;
C₃-C₁₇-heteroaryl,
which is optionally substituted with one or more C₁-C₅-alkyl substituents;
N(C₆-C₁₈-aryl)₂;
N(C₃-C₁₇-heteroaryl)₂; and
N(C₃-C₁₇-heteroaryl)(C₆-C₁₈-aryl); and
wherein any of the substituents R¹, R², R^{a}, R³, R⁴ or R⁵ independently from each other optionally form a mono- or polycyclic, aliphatic, aromatic and/or benzo-fused ring system with one or more other substituent R¹, R², R^{a}, R³, R⁴ or R⁵.

17. The organic molecule according to claim 16, comprising a structure of Formula Ib:

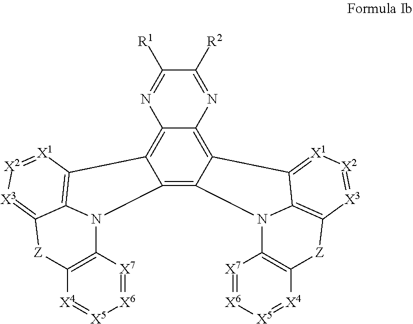

18. The organic molecule according to claim 16, wherein Z is a direct bond.

19. The organic molecule according to claim 16, wherein R¹ and R² is independently selected from the group consisting of:
phenyl, which is optionally substituted with one or more substituents R⁵; and
pyridine, which is optionally substituted with one or more substituents R⁵.

20. The organic molecule according to claim 16, wherein the substituents R² and R¹ are the same.

21. The organic molecule according to claim 17, comprising a structure selected from the group consisting of Formula IIIa, Formula IIIb, Formula IIIc, Formula IIId, Formula IIIe, Formula IIIf, Formula IIIg, Formula IIIh, and Formula IIIi:

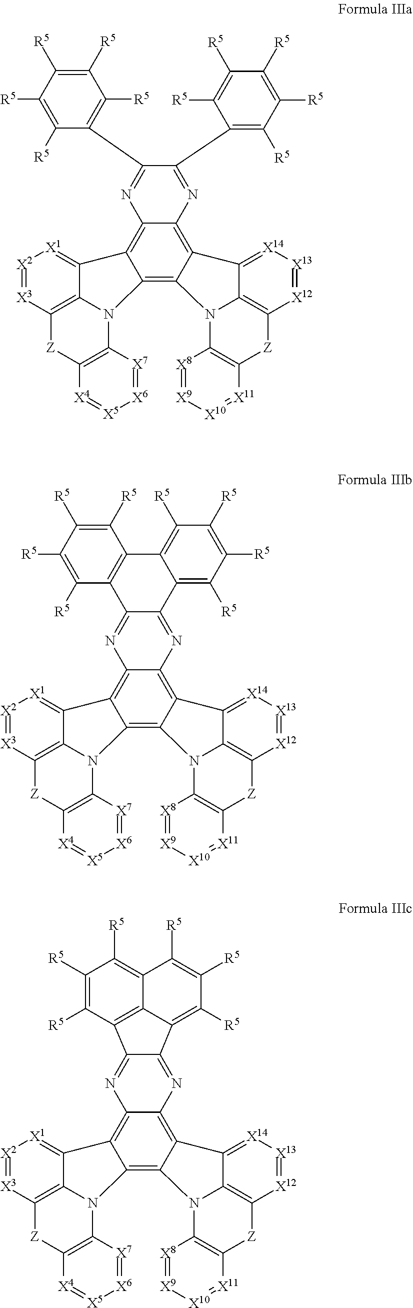

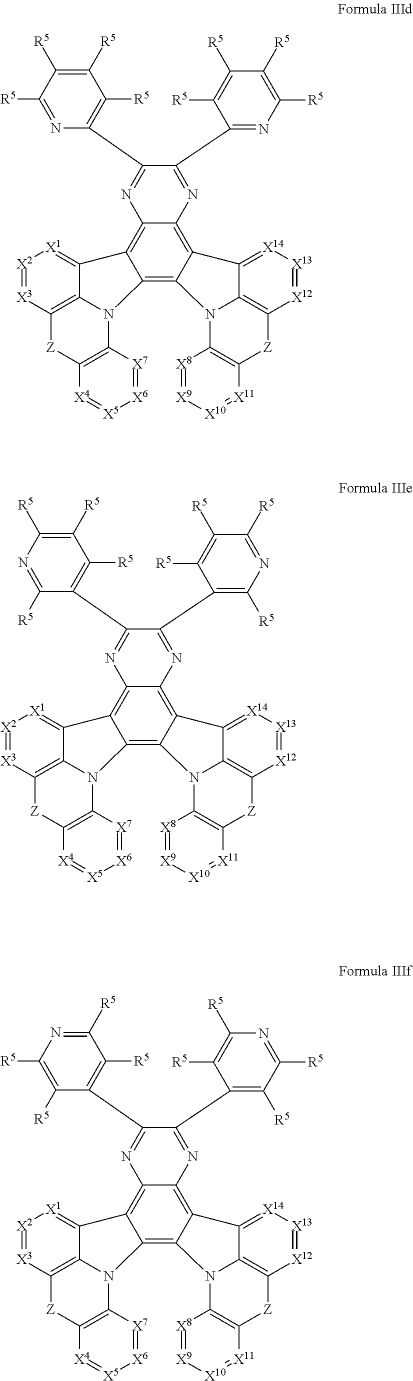

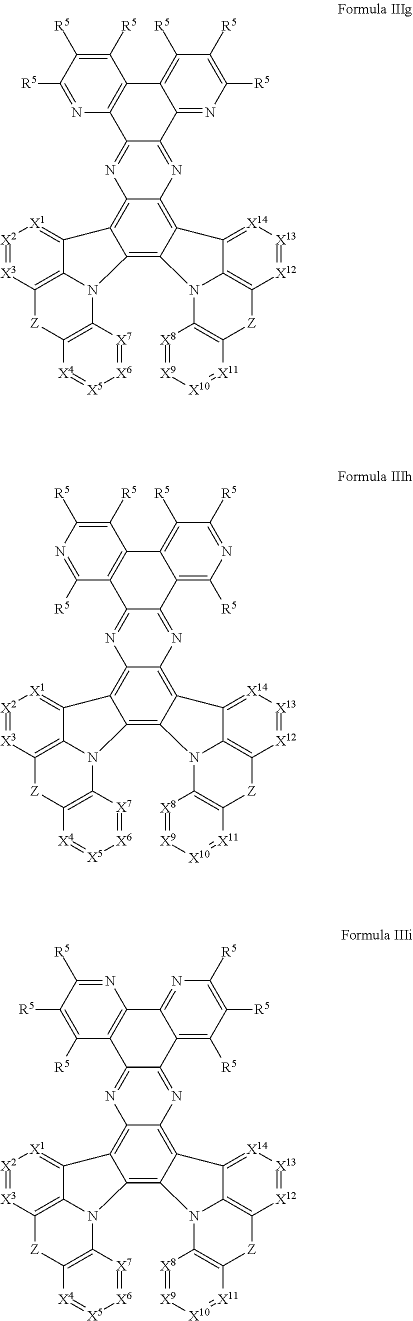

22. The organic molecule according to claim 16, wherein R^{a} is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

23. The organic molecule according to claim 16, wherein R⁵ is at each occurrence independently from one another selected from the group consisting of:
hydrogen,
Me,
ⁱPr,
^{t}Bu,
CN,
CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

24. An optoelectronic device comprising the organic molecule according to claim 16 as a luminescent emitter.

25. The optoelectronic device according to claim 24, wherein the optoelectronic device is at least one selected from the group consisting of:
organic light-emitting diodes (OLEDs),
light-emitting electrochemical cells,
OLED-sensors,
organic diodes,
organic solar cells,
organic transistors,
organic field-effect transistors,
organic lasers, and
down-conversion elements.

26. A composition, comprising:
(a) the organic molecule according to claim 16 as a luminescent emitter,
(b) a triplet-triplet annihilation (TTA) host material, which differs from the organic molecule,
(c) optionally, a thermally-activated delayed fluorescence (TADF) material, and
(d) optionally, a dye and/or a solvent.

27. An optoelectronic device, comprising the composition according to claim 26, wherein the optoelectronic device is at least one selected from the group consisting of organic light-emitting diodes (OLEDs), light-emitting electrochemical cells, OLED-sensors, organic diodes, organic solar cells, organic transistors, organic field-effect transistors, organic lasers, and down-conversion elements.

28. The optoelectronic device according to claim 24, comprising:
a substrate,
an anode, and
a cathode, wherein the anode or the cathode is on the substrate, and
a light-emitting layer between the anode and the cathode and comprising the organic molecule.

29. A method for producing an optoelectronic device, the method comprising depositing the organic molecule according to claim 16 by a vacuum evaporation method and/or a solution deposition method.

30. A method for producing an optoelectronic device, the method comprising depositing the composition according to claim 26 by a vacuum evaporation method and/or a solution deposition method.

31. The optoelectronic device according to claim 27, comprising:
a substrate,
an anode, and
a cathode, wherein the anode or the cathode is on the substrate, and
a light-emitting layer between the anode and the cathode and comprising the composition.

32. An optoelectronic device, comprising a layer formed from the composition according to claim 26, wherein the optoelectronic device is at least one selected from the group consisting of organic light-emitting diodes (OLEDs), light-emitting electrochemical cells, OLED-sensors, organic diodes, organic solar cells, organic transistors, organic field-effect transistors, organic lasers, and down-conversion elements.

33. The optoelectronic device according to claim 32, comprising:
a substrate,
an anode, and
a cathode, wherein the anode or the cathode is on the substrate, and
a light-emitting layer between the anode and the cathode and comprising the layer formed from the composition.
